(19) United States
(12) Patent Application Publication
(10) Pub. No.: US 2019/0224118 A1
(43) Pub. Date: Jul. 25, 2019

(54) ORAL GASTRORETENTIVE FORMULATIONS AND USES THEREOF

(71) Applicant: INTEC PHARMA LTD., Jerusalem (IL)

(72) Inventors: Nadav NAVON, Rehovot (IL); Ronny REINBERG, Modi'in (IL); Yochai YAKOVSON, Shilo (IL)

(21) Appl. No.: 16/316,390

(22) PCT Filed: Jul. 11, 2017
(86) PCT No.: PCT/IL2017/050783

§ 371 Date: Jan. 9, 2019

Related U.S. Application Data
(60) Provisional application No. 62/360,744, filed on Jul. 11, 2016.

(51) Int. Cl. A61K 9/00; A61K 31/352; A61K 31/05

(52) U.S. Cl. CPC A61K 9/0065; A61K 31/05; A61K 31/352

(57) ABSTRACT

Disclosed are gastro-retentive drug delivery devices and dosage units, for delivery of poorly water-soluble drugs, and methods of use thereof. Specific delivery devices and dosage forms are designed for delivery of cannabinoids.

### TECHNOLOGICAL FIELD

Disclosed are orally administered gastroretentive delivery systems for controlled and/or immediate delivery of poorly soluble drugs, including cannabis and cannabinoids, and their uses in medicine, including the treatment of various cannabinoid-responsive conditions.

### PRIOR ART

References considered to be relevant as background to the presently disclosed subject matter are listed below:
1. Information for Health Care Professionals—Cannabis (marihuana, marijuana) and the cannabinoids, Health Canada, Hanan Abramovici, February 2013
2. Borgelt, L. M. et al., Pharmacotherapy 2013 33(2): 195-209
3. Product information for AusPAR Nabiximols Sativex Australia Pty Limited PM-2011-00150-3-1, September 2013
4. Mechoulam, R., Mayo Clin Proc. 2012 February; 87(2): 107-109.
5. Klumpers, L. E. et al., Br. J. Clin. Pharmacol. 2012 July; 74(1): 42-53.
6. WO2007/083309
7. WO2009/144558
8. Zanchetta B., et al. (2015) J Adv Chem Eng 5: 130. doi:10.4172/2090-4568.1000130
9. Thakare P. et al., Pharmaceutical and Biological Evaluations 2016; 3(2): 140-153)
10. Kalepu S., et al. Acta Pharmaceutica Sinica B (2013) Volume 3, Issue 6, December 2013, Pages 361-372
11. Chen, Zhi-Qiang, et al. Int J Nanomedicine 7.1 (2012): 709
12. Zgair, Atheer, et al. American Journal of Translational Research, 8.8 (2016): 3448.
13. US 20150057342 A1
14. US 20070104741
15. U.S. Pat. No. 9,265,724 B2
16. WO2013009928 A1
17. U.S. Pat. No. 9,095,555 B
18. WO2011048494

These publications are referred to below by their above numbers. Reference is also made to publications referred to in the above listed publications in their entirety.

Acknowledgement of the above references herein is not to be inferred as meaning that these are in any way relevant to the patentability of the presently disclosed subject matter.

### BACKGROUND

### Gastro-Retentive Drug Delivery

Gastro-retentive controlled-release (CR) drug delivery systems for poorly soluble drugs have been described, for example in WO011048494 [18]. In such system, generally the drug is dispersed in a carrying polymer, forming a solid dispersion.

### Lipid-Based Drug Delivery

Poor drug absorption is commonly associated with active pharmaceutical ingredients having low aqueous solubility and/or poor intestinal permeability. Lipid-Based Drug Delivery (LBDD) is one approach used to improve drug solubilization in the dosage form and more importantly, in the gastrointestinal (GI) environment.

Self-Emulsifying Lipid Formulations (SELF) improve oral bioavailability of poorly water-soluble drugs by enhancing their solubility and maintaining the drug in a dissolved state, in small droplets of oil, during its transit through the GI tract. The improvement of the oral bioavailability has been attributed to increase of dissolution of the drug, larger surface area provided by the fine emulsion droplets, improved diffusion across the unstirred aqueous layer, and increased mucosal permeability due to high content of surfactants in the formulation, and also by the presence of long-chain oil that promotes lipoprotein synthesis with subsequent lymphatic absorption. The mechanisms by which these factors act are closely linked to the formulation components and properties of the emulsions formed, such as fast emulsification, mean size of the droplets and zeta potential.

Self-emulsification is the property of lipid Self-Emulsifying Drug Delivery Systems (SEDDS) that form emulsion particles upon contact with aqueous media without the need for mechanical or thermal energy. This can happen with multi-component excipients, or in formulations consisting of three distinct groups of molecules: oils, surfactants, and co-surfactants or solvents at optimal ratios. The right amount of each group is required for spontaneous formation of emulsion particles upon contact with aqueous media. The dispersion size of the SELF in aqueous media can be assessed by optical microscopy or dynamic/laser light scattering techniques. The naked eye can distinguish some of these particle ranges merely by the appearance (transparency, translucency or turbidity) [8].

### Oral Dosage Forms Containing Self-Emulsifying Formulations

Capsule filling is the simplest and most common technology for the encapsulation of liquid or semisolid self-emulsifying (SE) formulations for oral route delivery. Other solid SE dosage forms have emerged in recent years like incorporation of liquid/semisolid SE ingredients into powders/nanoparticles by different solidification techniques (e.g. adsorption to solid carriers, spray drying, melt extrusion, nano-particle technology). However SEDDS are usually limited to liquid dosage forms, because many excipients used in SEDDS are not solids at room temperature [9].

Examples of commercially available lipid-based products for oral administration are Agenerases® (GlaxoSmithKline), Rocaltrols® (Roche), Cipros® (Bayer), and several others [10]. Data clearly indicate that despite the multiple advantages and extensive research work in academia and industry, there are very few commercially successful products available in the market today.

### Polymers as Precipitation Inhibitors

The inclusion of certain polymers within solid dispersion or lipid-based formulations can maintain drug supersaturation after dispersion and/or digestion of the vehicle, leading to improvements in bioavailability and variability in exposure.

An emulsion can be formed upon dispersion of SEDDS in aqueous solution. However, when a drug is released from an emulsion, precipitation often occurs due to decreased solubility, leading to decreased drug dissolution and absorption in vivo. Thus, inhibiting drug precipitation upon mixing SMEDDS with aqueous solution is a key consideration in designing these formulations.

A supersaturation process can maintain drug solubilization above equilibrium solubility without precipitation. A high energy form of the drug (in comparison with crystalline powder) in solution yields a supersaturated state with increased chemical potential. Thus, it is a thermodynamically unstable system. When a supersaturated drug delivery system exists at the absorption site for a sufficient period of time, the higher drug concentration generated from the supersaturated state may enhance drug absorption. Hydrophilic polymers such as hydroxypropyl methylcellulose (HPMC) and polyvinylpyrrolidone (PVP) can be used in Self-Emulsifying Drug Delivery Systems (SEDDS) and Self-Microemulsifying Drug Delivery Systems (SEDDS) formulations as precipitation inhibitors to form super-saturatable self-emulsifying drug delivery systems.

When SEDDS or SMEDDS come into contact with the aqueous environment of the GI tract, the preparations are first emulsified, and an emulsion or microemulsion is formed immediately. The drug may be dissolved in free form or incorporated in emulsion or microemulsion droplets. Precipitation inhibitors may increase the solubility of the free drug or the drug in the microemulsion and further increase the concentration gradient of the drug across the intestinal membrane, which may significantly improve the water solubility of the drug and enhance oral absorption [11].

### Cannabinoids

The principal cannabinoids in cannabis are Δ-9-tetrahydrocannabinol (Δ9-THC, THC), cannabinol (CBN), cannabidiol (CBD) cannabigerol (CBG), cannabichromene (CBC), tetrahydrocannabivarin (THCV) and many others. The relative abundance of these and other cannabinoids can vary depending on a number of factors such as the Cannabis strain, the soil and climate conditions, and the cultivation techniques [1].

### Medical Use

Medical uses of cannabis and cannabinoids include both studied approved uses and off-label uses [1-4]. These medical uses include, inter alia, treatment of anorexia associated with weight loss in patients with AIDS, nausea and vomiting associated with cancer chemotherapy, pain, anxiety, depression, muscle spasticity, arthritis and rheumatism, multiple sclerosis and other neuromuscular inflammatory disorders, inflammatory bowel diseases such as Crohn's disease and colitis, post-traumatic stress disorder (PTSD) and epileptic seizures.

The benefits of medical cannabis can be attributed to binding to the endocannabinoid system. This has many effects including modulating the immune system, promoting neuroplasticity, emotional and cognitive modulation including learning and motivation, appetite, vascular function, and digestive function. Cannabidiol (CBD) affects the activity of a significant number of other targets including ion channels, receptors, and enzymes [1]. Results from pre-clinical studies suggest CBD has anti-inflammatory, analgesic, anti-nausea, anti-emetic, anti-psychotic, anti-ischemic, anxiolytic, and anti-epileptiform effects [1].

### Lipid Based Formulations of Cannabinoids

The use of dietary fats and pharmaceutical lipid-based excipients is common in the preparation of cannabis-containing foods and cannabis-based medicinal formulations. The vast majority of cannabis-cooking recipes involve the use of dietary lipids (whole milk, butter, or vegetable oil) for the preparation of these cannabis-containing foods. Oral formulations of THC and CBD (Marinol® and Epidiolex®, respectively) contain sesame oil, which is mostly composed of long-chain triglycerides.

### Methods of Use

### Smoked Cannabis

Smoking cannabis results in more rapid onset of action (within minutes), higher blood levels of cannabinoids, and a shorter duration of pharmacodynamic effects compared to oral administration. Smoking cannabis is the most abundant form of administration and is effective for acute states of disease. While onset of effect is fast, effective levels decline is also quick, such that the relief effect does not last [1].

The amount of cannabinoids absorbed/delivered from cannabis cigarettes is not uniform and is variable, and depends on the source of the plant material and the composition of the cigarette, together with the efficiency and method of smoking used by the subject, depth of inhalation, puff duration, and breath hold. This lack of controlled dosing may reduce clinical efficacy or induce side effects, and may also occur after vaporization of cannabis or THC.

Vaporization of cannabis [1] or inhaled cannabis have been explored as an alternative to smoking. The potential advantages of vaporization include the formation of a smaller quantity of toxic by-products such as carbon monoxide, polycyclic aromatic hydrocarbons (PAHs), and tar, as well as a more efficient extraction of Δ9-THC from the cannabis material.

Oral

Oral use of cannabis or cannabinoids either by administration of oral dosage forms (capsules, tablets) or by ingestion of foods containing cannabis (e.g. butters, oils, brownies, cookies and the like) results in a slower onset of action, lower peak blood levels of cannabinoids and/or their active metabolites, and a longer duration of pharmacodynamic effects compared to smoking [1].

For orally administered prescription cannabinoid medicines such as synthetic Δ9-THC (dronabinol, marketed as Marinol®), only about 10-20% of the administered dose enter the systemic circulation due to extensive first-pass metabolism and the poor water solubility of THC [1]. Other disadvantages of current administration forms are the long Tₘₐₓ-values for these formulations, ranging from 1 to 4 h for Marinol® and Cesamet® (nabilone). Long time to reach a maximal concentration can be a disadvantage for on demand symptomatic treatment. Oral dronabinol formulations, such as Marinol®, have variable pharmacokinetics, as peak plasma concentration variations from 150% to 200% were observed in previous studies [1].

### Oro-Mucosal

Following a single oro-mucosal administration of nabiximols (Sativex®) (four sprays totaling 10.8 mg Δ9-THC and 10 mg CBD), mean peak plasma concentrations of both THC (˜5.5 ng/mL) and CBD (˜3 ng/mL) typically occur within 2-4 hours, although there is wide inter-individual variation in the peak cannabinoid plasma concentrations and in the time to onset and peak of effects [1].

When administered oro-mucosally, blood levels of Δ9-THC and other cannabinoids were lower than those achieved by inhalation of the same dose of smoked cannabis, but Δ9-THC blood levels were comparable to those seen with oral administration of dronabinol [1].

### Topical

Cannabinoids are highly hydrophobic, making transport across the aqueous layer of the skin the rate-limiting step in the diffusion process [1]. No clinical studies exist regarding the percutaneous absorption of cannabis-containing ointments, creams, or lotions. However, some research has been carried out on transdermal delivery of synthetic and natural cannabinoids using a dermal patch [1].

### Metabolism

Most cannabinoid metabolism occurs in the liver and different metabolites predominate depending on the route of administration.

Δ9-THC is oxidized by the xenobiotic-metabolizing cytochrome P450 (CYP) mixed-function oxidases 2C9, 2C19, and 3A4. The major initial metabolites (hepatic first pass) of Δ9-THC are its active metabolite 11-hydroxy Δ9-THC, and the non-active 11-nor-9-carboxy Δ9-THC (THC-COOH), the most abundant metabolite in human plasma and urine.

After administration of oral doses of Δ9-THC, THC and 11-hydroxy-Δ9-THC are present in the plasma in approximately equal concentrations. The plasma levels of active 11-hydroxy metabolite, achieved through oral administration, are about three times higher than those seen with smoking. Two THC peaks frequently were observed due to enterohepatic circulation. CBD undergoes hepatic first pass metabolism to 7-OH-CBD. CBD is extensively metabolized and more than 33 metabolites have been identified in urine.

### Short Half-Life

The elimination of oral cannabinoids from plasma is bi-phasic with an initial half-life of approximately four hours.

The elimination phase of synthetic THC (dronabinol MARINOL®) can be described using a two compartment model with an initial (alpha) half-life of about 2-4 hours.

From clinical studies with Sativex® (oromucosal spray, each 100 microliter spray containing 2.7 mg THC and 2.5 mg CBD), a non-compartmental PK analysis shows that the first order terminal elimination half-life from plasma following administration of total 5 or 10 mg THC and CBD is 1.94 or 3.72 hours, respectively, for THC and 5.28 or 6.39 hours, respectively, for CBD.

A study that compared the pharmacokinetic parameters of THC after sublingual and oral formulation (Namisol®) found that the half-life of THC was 279 minutes for the sublingual formulation and 196-318 minutes for the oral formulation [5].

Based on the above, current methods of use and treatment with cannabis suffer from major drawbacks, including short duration of effect, delayed onset (lag time), low bioavailability, variability of exposure, dose variability, narrow therapeutic window and adverse events that correlates with peak levels, adverse effects related to the method of use like mouth wounds, bad taste and adverse effect related to smoking and frequent daily dosing.

### SUMMARY

In a first aspect, disclosed herein is a gastro-retentive drug delivery device for oral administration, the device being configured for unfolding from a folded configuration for oral intake to an unfolded configuration for gastric retention, the device comprising (a) a drug-containing layer comprising a polymeric carrier, said carrier comprising at least one film-forming polymer and at least one emulsified drug; and (b) a polymeric frame member configured for imparting mechanical strength to the device sufficient to enable, upon unfolding of the device, the preservation of said unfolded configuration to provide gastric retention, said polymeric frame member accommodating said emulsified drug-containing layer; and (c) one or two polymeric swelling membranes each covering at least in part one of the two faces of the emulsified drug-containing layer accommodated within said frame member, at least one said swelling membranes optionally comprising orifices.

In embodiments of the said gastro-retentive drug delivery device, said at least one emulsified one drug can be in the form of an emulsion of said drug in a pharmaceutically acceptable emulsifying agent. The said emulsifying agent can be at least one oil, glyceride, water insoluble surfactant, water soluble surfactant or co-solvent or any mixture of at least two thereof.

In the said gastro-retentive drug delivery device, the weight ratio between said film forming polymer and said emulsion can be from about 1:2 to about 20:1. The weight ratio between said at least one pharmaceutically active drug and said emulsifying agent can be from about 2:1 to about 1:20.

In embodiments of the said gastro-retentive drug delivery device, said at least one drug has log P>2.

In specific embodiments of the said gastro-retentive drug delivery device, said at least one drug is a pharmaceutically active cannabinoid or a mixture of at least two pharmaceutically active cannabinoids or a pharmaceutically active cannabis extract.

In some embodiments, the said gastro-retentive drug delivery device further optionally comprises at least one emulsified drug-containing polymeric layer for immediate release (IR) of said at least one drug (IR layer) covering at least in part one said swelling membrane, said at least one IR layer comprising (1) at least one pharmaceutically acceptable film forming polymer and (2) at least one pharmaceutically active emulsified drug. In some such embodiments, the said gastro-retentive drug delivery device comprises two drug-containing IR layers, each said IR layer covering at least in part one said swelling membrane.

In some specific embodiments, in the said gastro-retentive drug delivery device said at least one pharmaceutically active emulsified drug and said at least one film forming polymer can be distributed essentially homogeneously throughout the said polymeric carrier. In additional embodiments, in the said gastro-retentive drug delivery device said at least one pharmaceutically active emulsified drug comprised in said at least one IR layer and said at least one film forming polymer comprised in said at least one IR layer can be distributed essentially homogeneously throughout said at least one IR layer.

In embodiments of the said gastro-retentive drug delivery device said at least one film forming polymer can be selected from polymers that are water-soluble and polymers that are partially or completely soluble in both water and organic solvents, and any mixture of at least two thereof. In the said gastro-retentive drug delivery device said polymeric carrier further optionally comprises at least one of a pharmaceutically acceptable plasticizer and a pharmaceutically acceptable antioxidant. In addition, said polymeric carrier can further comprise at least one pharmaceutically acceptable swelling polymer. The said at least one film forming polymer can be any one of povidone, copovidone, hydroxypropyl cellulose, polyethylene oxide, amino-methacrylate copolymer NF, hydroxypropyl methylcellulose, hydroxyethyl cellulose, carboxymethyl cellulose polyvinyl alcohol-polyethylene glycol graft copolymer and any combination of at least two thereof. The said plasticizer can be any one of polyethylene glycols, citrate esters, phthalate esters, glyceryl esters, short-chain triglycerides, medium-chain triglycerides, long-chain triglycerides, olive oil, hydrogenated castor oil, triacetin, glyceryl stearate, glyceryl behenate, dibutyl sebacate, aliphatic alcohols, fatty acids, pegylated aliphatic alcohols and pegylated fatty acids, phospholipids, sorbitan derivatives, polysorbates, poloxamers, hydrogenated castor oil derivatives, glycerin, propylene glycol, and a combination of at least two thereof. The said swelling polymer can be any one of hydroxypropyl methylcellulose, hydroxypropyl cellulose, hydroxyethyl cellulose, polyethylene oxide, carboxymethyl cellulose, a gum, a protein, and any combination of at least two thereof.

In the said gastro-retentive drug delivery device said at least one IR layer can further comprise at least one of a filler, a surface-active material, a disintegrant, antioxidant or a combination of any two thereof. said two IR layers further comprise at least one material that is a plasticizer, a filler, a surface-active material, disintegrant, antioxidant, or any combination of at least two thereof. Said plasticizer in said IR layers can be any one of a polyethylene glycols, citrate esters, phthalate esters, glyceryl esters, short-chain triglycerides, medium-chain triglycerides, long-chain triglycerides, olive oil, hydrogenated castor oil, triacetin, glyceryl stearate, glyceryl behenate, dibutyl sebacate, aliphatic alcohols, fatty acids, pegylated aliphatic alcohols and pegylated fatty acids, phospholipids, sorbitan derivatives, polysorbates, poloxamers, hydrogenated castor oil derivatives, glycerin, propylene glycol, and a combination of at least two thereof. Said disintegrant in said IR layers can be any one of microcrystalline cellulose, crospovidone, croscarmellose, starch and its derivatives, polacrilin, or a mixture of any two thereof.

In the said gastro-retentive drug delivery device said swelling membranes can each comprise at least one polymeric combination of a soluble polymer and a polymer which is not instantly soluble in gastric medium. Said soluble polymer can be any one of hydroxypropyl cellulose, gelatin, hydroxypropylmethyl cellulose, hydroxyethyl cellulose, carboxymethyl cellulose and polyethylene oxide. Said polymer which is not instantly soluble in gastric fluid comprised in said swelling membrane can be any one of methacrylic acid copolymer NF, cellulose acetate phthalate, hydroxypropyl methylcellulose phthalate, hydroxypropyl methylcellulose acetate succinate, polyvinyl acetate phthalate or any suitable mixture of at least two thereof.

In the said gastro-retentive drug delivery device said polymeric frame member can comprise at least one polymer that is not instantly soluble in gastric fluid, which can be a degradable enteric polymer which is substantially insoluble at pH less than 5.5. The said polymer that is not instantly soluble in gastric fluid comprised in said polymeric frame member is any one of cellulose acetate phthalate, hydroxypropyl methylcellulose phthalate, hydroxypropyl methylcellulose acetate succinate, polyvinyl acetate phthalate and methacrylic acid copolymer NF, and any suitable mixture of at least two thereof.

The said frame member can further comprise a plasticizer. The said plasticizer can be any one of a polyethylene glycol, or a mixture of two or more polyethylene glycols of different molecular weight, such as any of PEG 200, PEG 300, PEG 400, PEG 540, PEG 600, PEG 800, PEG 1000, PEG 1450, PEG 1540, PEG 3350, PEG 4000, PEG 4500, PEG 6000 and PEG 8000 and PEG 20000, and wherein said plasticizer optionally includes a poloxamer, medium-chain triglycerides, glycerin, glyceryl esters, a polysorbate, a sorbitan derivative, citric acid esters, dibutyl sebacate, an aliphatic alcohol, such as cetyl alcohol, a fatty acid, such as stearic acid, propylene glycol or a combination of the above, preferably the plasticizer is a polyethylene glycol, and a mixture of two or more PEGs with different molecular weight thereof, for example a mixture of PEG 400 and PEG 20,000.

In embodiments of the said gastro-retentive drug delivery device, said orifices are provided on one of said swelling membranes. In other embodiments, said orifices are provided on both said swelling membranes.

In embodiments of the said gastro-retentive drug delivery device, said device can further comprise an anti-adhesion layer covering at least one said swelling membrane. In additional embodiments, the said gastro-retentive drug delivery device can further comprise an anti-adhesion layer covering at least in part said at least one IR layer.

In embodiments of the said gastro-retentive drug delivery device, said at least one swelling membrane comprises a suitable number of identical or different said orifices, and each said orifice has one or more of suitable dimensions, suitable distribution pattern and/or suitable shape. For example, said orifices can be uniformly distributed over the respective at least one said swelling membranes. In other embodiments, said orifices are provided on both said swelling membranes and said orifices of one said swelling membrane are staggered with respect to said orifices of the other said swelling membrane. Each said swelling membrane can comprise, for example, from 2 to 24, specifically from 8 to 24 of said orifices. Each said orifice can have a diameter or width of between 0.3 mm and 2.5 mm.

In embodiments of the said gastro-retentive drug delivery device, said two swelling membranes are co-extensive with said drug-containing layer.

The said emulsified drug is released from the said gastro-retentive drug delivery device in emulsified form.

In embodiments of the said gastro-retentive drug delivery device of the first aspect of the present disclosure, said drug is a pharmaceutically active cannabinoid or a mixture of at least two pharmaceutically active cannabinoids or a pharmaceutically acceptable cannabis extract.

In some embodiments, the said gastro-retentive drug delivery device, in the said folded configuration is folded into a plurality of pleats via folds, each fold being defined between an adjacent pair of said pleats. Particularly in its said folded configuration, said gastro-retentive drug delivery device can be contained within a capsule.

In a second aspect of the present disclosure, disclosed herein is a pharmaceutical dosage unit comprising a gastro-retentive drug delivery device as disclosed herein in said first aspect, and a capsule, wherein said drug delivery device in its folded configuration is contained within said capsule. In specific embodiments, for example where said device does not comprise IR layer/s, said capsule can further contain an emulsion of said at least one drug in a pharmaceutically acceptable emulsifying agent. The said dosage unit, comprises a therapeutically effective amount of said at least one emulsified drug, comprised in said device and, where present in said emulsion further contained in said capsule. In some embodiments said emulsified drug is at least one of emulsified pharmaceutically active cannabinoid or pharmaceutically active cannabis extract.

In the disclosed pharmaceutical dosage unit of said second aspect, the delivery device can comprise a total of from about 1 to about 350 mg of said at least one pharmaceutically active cannabinoid or mixture of at least two pharmaceutically active cannabinoids or pharmaceutically active cannabis.

In the said pharmaceutical dosage unit, said at least one emulsified pharmaceutically active cannabinoid or emulsified mixture of at least two pharmaceutically active cannabinoids or pharmaceutically active cannabis extract can be distributed between said polymeric carrier and said at least one IR layer or where present, said emulsion of said cannabinoid/s in said oil contained in said capsule.

In the said pharmaceutical dosage unit, said emulsified mixture of at least two pharmaceutically active cannabinoids can comprise THC and CBD at a ratio of from about 20:1 to about 1:20. The weight ratio between said film forming polymer and said at least one emulsified pharmaceutically active cannabinoid or pharmaceutically active cannabis extract is from about 1:2 to about 20:1. The ratio between said pharmaceutically active cannabinoid or mixture of at least pharmaceutically active two cannabinoids or pharmaceutically active cannabis extract and the emulsifying agent in which they are emulsified can be between 2:1 to 1:20.

In embodiments of the said pharmaceutical dosage unit, the delivery device can comprise a total of from about 1 to about 350 mg of a mixture of THC and CBD, distributed between said polymeric carrier and said at least one IR layer at a ratio of from about 1:10, to about 10:1, the ratio THC:CBD in said polymeric carrier and in said at least one IR layer which can be the same or different being from about 1:20 to about 20:1.

In some embodiments of the said pharmaceutical dosage unit, said polymeric carrier can comprise one specific cannabinoid, or a mixture of at least two specific cannabinoids, at a suitable ratio therebetween, and said at least one IR layer, respectively said drug emulsion in said capsule, can comprise the same or different one specific cannabinoid or mixture of said at least two specific cannabinoids at a suitable ratio therebetween, the ratio between the at least two cannabinoids in said polymeric carrier and in said at least one IR layer, respectively said drug emulsion in said capsule, being the same or different.

In a further, third aspect, disclosed herein is a pharmaceutical dosage unit for oral administration of a pharmaceutically active cannabinoid or a mixture of at least two pharmaceutically active cannabinoids or cannabis extract, comprising: (A) a gastro-retentive cannabinoid delivery device, the device being configured for unfolding from a folded configuration for oral intake to an unfolded configuration for gastric retention, the device comprising (a) a cannabinoid-containing layer comprising a polymeric carrier, said carrier comprising at least one film forming polymer and at least one pharmaceutically active cannabinoid or cannabinoid-releasing extract formulation; and (b) a polymeric frame member configured for imparting mechanical strength to the device sufficient to enable, upon unfolding of the device, the preservation of said unfolded configuration to provide gastric retention, said polymeric frame member accommodating said cannabinoid-containing layer; and (c) one or two polymeric swelling membranes each covering at least in part one of the two faces of the cannabinoid-containing layer accommodated within said frame member, at least one said swelling membranes optionally comprising orifices; and (B) a capsule; wherein said cannabinoid delivery device in its folded configuration is contained in said capsule.

In another fourth aspect of the present disclosure, disclosed is pharmaceutical dosage unit for oral administration of a pharmaceutically active cannabinoid or a mixture of at least two pharmaceutically active cannabinoids or cannabis extract, comprising: (A) a gastro-retentive cannabinoid delivery device, the device being configured for unfolding from a folded configuration for oral intake to an unfolded configuration for gastric retention, the device comprising (a) a cannabinoid-containing layer comprising a polymeric support which comprises at least one suitable polymer selected from degradable hydrophilic polymers which is not instantly soluble in gastric fluid, degradable enteric polymers substantially insoluble at pH less than 5.5, or any mixture thereof, and at least one pharmaceutically active cannabinoid or cannabinoid-releasing formulation, wherein the polymeric support is configured for imparting mechanical strength to the device sufficient to enable, upon unfolding of the device, the preservation of said unfolded configuration to provide gastric retention; and (b) one or two polymeric swelling membranes each covering at least in part one of the two faces of the cannabinoid-containing layer, at least one said swelling membranes optionally comprising orifices; and (B) a capsule; wherein said cannabinoid delivery device in its folded configuration is contained in said capsule.

In some embodiments, the said pharmaceutical dosage units according to said third or fourth aspects of the present disclosure, further optionally comprise at least one cannabinoid-containing polymeric layer for immediate release (IR) of the cannabinoid/s (IR layer) covering at least in part one said swelling membrane, said at least one IR layer comprising (1) at least one pharmaceutically acceptable film forming polymer and (2) at least one pharmaceutically active cannabinoid or cannabinoid-releasing formulation. In some embodiments, the said pharmaceutical dosage units, comprise two said cannabinoid-containing IR layers, each said IR layer covering at least in part one said swelling membrane.

In embodiments of the pharmaceutical dosage unit according to said third or fourth aspects of the present disclosure, said at least one pharmaceutically active cannabinoid or cannabis extract and said at least one film forming polymer are distributed essentially homogeneously throughout the said polymeric carrier, respectively throughout the said polymeric support. In some embodiments, the said pharmaceutically active cannabinoid or cannabis extract and said at least one film forming polymer are distributed essentially homogeneously throughout said at least one IR layer.

In embodiments of the pharmaceutical dosage unit according to said third or fourth aspects of the present disclosure, said at least one film forming polymer is selected from polymers that are water-soluble and polymers that are partially or completely soluble in both water and organic solvents, and any mixture of at least two thereof.

In embodiments of the pharmaceutical dosage unit according to said third or fourth aspects of the present disclosure, said polymeric carrier, respectively said polymeric support further optionally comprises at least one of a pharmaceutically acceptable plasticizer, antioxidant, solubilizer and a pharmaceutically acceptable basic substance or alkaline agent, such as a pharmaceutically acceptable metal hydroxide, salt or buffer.

In embodiments of the pharmaceutical dosage unit according to said third or fourth aspects of the present disclosure, said IR layer can further comprise a plasticizer, which is identical or different from said plasticizer comprised in said polymeric carrier, respectively polymeric support.

In embodiments of the pharmaceutical dosage unit according to said third or fourth aspects of the present disclosure, said polymeric carrier, respectively polymeric support, can further comprise at least one pharmaceutically acceptable swelling polymer.

In embodiments of the pharmaceutical dosage unit according to said third or fourth aspects of the present disclosure, the said at least one film forming polymer is any one of povidone, copovidone, hydroxypropyl cellulose, polyethylene oxide, amino-methacrylate copolymer NF, hydroxypropyl methylcellulose, hydroxyethyl cellulose, carboxymethyl cellulose polyvinyl alcohol-polyethylene glycol graft copolymer and any combination of at least two thereof.

In embodiments of the pharmaceutical dosage unit according to said third or fourth aspects of the present disclosure, said plasticizer can be any one of polyethylene glycols, citrate esters, phthalate esters, glyceryl esters, short-chain triglycerides, medium-chain triglycerides, long-chain triglycerides, olive oil, hydrogenated castor oil, triacetin, glyceryl stearate, glyceryl behenate, dibutyl sebacate, aliphatic alcohols, fatty acids, pegylated aliphatic alcohols and pegylated fatty acids, phospholipids, sorbitan derivatives, polysorbates, poloxamers, hydrogenated castor oil derivatives, glycerin, propylene glycol, and a combination of at least two thereof.

In embodiments of the pharmaceutical dosage unit according to said third or fourth aspects of the present disclosure, the said swelling polymer can be any one of a hydroxypropyl methylcellulose, a hydroxypropyl cellulose, hydroxyethyl cellulose, a polyethylene oxide, a carboxymethyl cellulose, a gum, a protein, and any combination of at least two thereof.

In embodiments of the pharmaceutical dosage unit according to said third or fourth aspects of the present disclosure, the said IR layer can further comprise at least one of a filler, surface-active material, disintegrant, antioxidant, lipid, or a combination of any two thereof.

In embodiments of the pharmaceutical dosage unit according to said third or fourth aspects of the present disclosure, the said swelling membranes can each comprise at least one polymeric combination of a soluble polymer and a polymer which is not instantly soluble in gastric medium. The said soluble polymer can be any one of hydroxypropyl cellulose, gelatin, hydroxypropylmethyl cellulose, hydroxyethyl cellulose, carboxymethyl cellulose and polyethylene oxide. The said polymer which is not instantly soluble in gastric fluid comprised in said swelling membrane can be any one of methacrylic acid copolymer NF, cellulose acetate phthalate, hydroxypropyl methylcellulose phthalate, hydroxypropyl methylcellulose acetate succinate, polyvinyl acetate phthalate or any suitable mixture of at least two thereof.

In embodiments of the pharmaceutical dosage unit according to said third or fourth aspects of the present disclosure, the said two IR layers can further comprise at least one material that is a plasticizer, a filler, a surface-active material, disintegrant, a lipid, or a combination of at least two thereof. The said plasticizer in said IR layers can be any one of a polyethylene glycols, citrate esters, phthalate esters, glyceryl esters, short-chain triglycerides, medium-chain triglycerides, long-chain triglycerides, olive oil, hydrogenated castor oil, triacetin, glyceryl stearate, glyceryl behenate, dibutyl sebacate, aliphatic alcohols, fatty acids, pegylated aliphatic alcohols and pegylated fatty acids, phospholipids, sorbitan derivatives, polysorbates, poloxamers, hydrogenated castor oil derivatives, glycerin, propylene glycol, and a combination of at least two thereof. The said disintegrant in said IR layers can be any one of microcrystalline cellulose, crospovidone, croscarmellose, starch and its derivatives, polacrylin, or a mixture of any two thereof. The said lipid can be any one of a polysorbate, a sorbitan derivative, sodium lauryl sulphate, hydrogenated castor oil and its derivatives or a triglyceride.

In embodiments of the pharmaceutical dosage unit according to said third aspect of the present disclosure, the said polymeric frame member can comprise at least one polymer that is not instantly soluble in gastric fluid, for example a degradable enteric polymer which is substantially insoluble at pH less than 5.5. The said polymer that it not instantly soluble in gastric fluid comprised in said polymeric frame member can be, for example, any one of cellulose acetate phthalate, hydroxypropyl methylcellulose phthalate, hydroxypropyl methylcellulose acetate succinate, polyvinyl acetate phthalate and methacrylic acid copolymer NF, and any suitable mixture of at least two thereof. A specific example is a methacrylic acid copolymer NF.

In embodiments of the pharmaceutical dosage unit according to said third aspect of the present disclosure, the said frame member can further comprise a plasticizer, for example, any one of a polyethylene glycol, or a mixture of two or more polyethylene glycols of different molecular weight, such as any of PEG 200, PEG 300, PEG 400, PEG 540, PEG 600, PEG 800, PEG 1000, PEG 1450, PEG 1540, PEG 3350, PEG 4000, PEG 4500, PEG 6000 and PEG 8000 and PEG 20000, and wherein said plasticizer optionally includes a poloxamer, medium-chain triglycerides, glycerin, glyceryl esters, a polysorbate, a sorbitan derivative, citric acid esters, dibutyl sebacate, an aliphatic alcohol, such as cetyl alcohol, a fatty acid, such as stearic acid, propylene glycol or a combination of the above, preferably the plasticizer is a polyethylene glycol, and a mixture of two or more PEGs with different molecular weight thereof, for example a mixture of PEG 400 and PEG 20,000.

In embodiments of the pharmaceutical dosage unit according to said fourth aspect of the present disclosure, the said degradable hydrophilic polymer which is not instantly soluble in gastric fluid comprised in said polymeric support can be any one of hydroxypropyl cellulose, hydroxypropylmethyl cellulose, hydroxyethyl cellulose carboxymethyl cellulose, polyvinyl pyrrolidone, polyethylene oxide and methylcellulose. The said degradable enteric polymer substantially insoluble at pH less than 5.5 comprised in said polymeric support can be any one of polymethacrylate copolymer, cellulose acetate phthalate, hydroxypropylmethyl cellulose acetate succinate or hydroxypropylmethyl cellulose phthalate. The said polymeric support can further comprise a filler, a disintegrant, a surface-active agent, an additional plasticizer and at least one other processing aid.

In embodiments of the pharmaceutical dosage unit according to said third or fourth aspects of the present disclosure, said orifices are provided on one of said swelling membranes or on both said swelling membranes. Each said at least one swelling membrane can comprise a suitable number of identical or different said orifices, each said orifice having one or more of suitable dimensions, suitable distribution pattern and/or suitable shape. The said orifices can be uniformly distributed over the respective at least one said swelling membranes. In some embodiments said orifices can be provided on both said swelling membranes and wherein in said orifices of one said swelling membrane are staggered with respect to said orifices of the other said swelling membrane. In some embodiments, each said swelling membrane can comprise from 2 to 24, specifically from 8 to 24 of said orifices. Each said orifice can have a diameter or width of between 0.3 mm and 2.5 mm.

In embodiments of the pharmaceutical dosage unit according to said third or fourth aspects of the present disclosure, said device further comprises an anti-adhesion layer covering at least one said swelling membrane. The said device further can comprise an anti-adhesion layer covering at least in part said at least one IR layer.

In embodiments of the pharmaceutical dosage unit according to said third or fourth aspects of the present disclosure, wherein said two swelling membranes are co-extensive with said cannabinoid-containing layer.

In embodiments of the pharmaceutical dosage unit according to said third or fourth aspects of the present disclosure, the said capsule is configured for disintegrating in a gastric environment on exposure thereto.

In embodiments of the pharmaceutical dosage unit according to said third or fourth aspects of the present disclosure, wherein the delivery device comprises a total of from about 1 to about 350 mg of said pharmaceutically active cannabinoid or mixture of at least two cannabinoids.

In embodiments of the pharmaceutical dosage unit according to said third or fourth aspects of the present disclosure, the delivery device can comprise a total of from about 1 to about 350 mg of said pharmaceutically active cannabinoid or mixture of at least two cannabinoids or said cannabis extract, distributed between said polymeric carrier, respectively polymeric support and said at least one IR layer. The said pharmaceutically active mixture of at least two cannabinoids can comprises THC and CBD at a ratio of from about 1:20 to about 20:1. In some specific embodiments, the delivery device can comprise a total of from about 1 to about 350 mg of a mixture of THC and CBD, distributed between said polymeric carrier or polymeric support and said at least one IR layer at a ratio of from about 1:10 to about 10:1, wherein the ratio THC:CBD in said polymeric carrier or polymeric support and in said at least one IR layer is the same or different.

In some embodiments of the pharmaceutical dosage unit according to said third or fourth aspects of the present disclosure, said polymeric carrier, respectively or polymeric support, can comprise one defined cannabinoid, for example THC or CBD, or a defined mixture of at least two cannabinoids, for example THC and CBD at a suitable ratio therebetween, and said at least one IR layer comprises the same or different one defined cannabinoid or defined mixture of at least two cannabinoids at a suitable ratio therebetween, wherein the ratio between the at least two cannabinoids in said polymeric carrier or polymeric support and in said at least one IR layer is the same or different.

In embodiments of the pharmaceutical dosage unit according to said third aspect of the present disclosure, said polymeric carrier can comprise three distinct contiguous laminated polymeric films, a first polymeric film comprising at least one cannabinoid, a second polymeric film comprising at least one cannabinoid and a third polymeric film being a non-drug-containing polymeric film, wherein said third polymeric film is positioned between said first and second polymeric films, and wherein said at least one cannabinoid comprised in said first polymeric film and said at least one cannabinoid comprised in said second polymeric film are the same or different. Each said first and second polymeric films can release said at least one cannabinoid or cannabis extract comprised therein at a controlled release rate, wherein the controlled release rates of said at least one cannabinoid from each said first and second cannabinoids are the similar or different rates of release.

In embodiments of the pharmaceutical dosage unit according to said fourth aspect of the present disclosure, said polymeric support can comprise three distinct contiguous laminated polymeric films, a first polymeric film comprising at least one cannabinoid, a second polymeric film comprising at least one cannabinoid and a third polymeric film being an inert non-drug-containing polymeric film, wherein said third polymeric film is positioned between said first and second polymeric films, and wherein said at least one cannabinoid comprised in said first polymeric film and said at least one cannabinoid comprised in said second polymeric film are the same or different. Each said first and second polymeric films can release said at least one cannabinoid comprised therein at a controlled release rate, wherein the controlled release rates of said at least one cannabinoid from each said first and second cannabinoids are the similar or different rates of release.

In embodiments of the pharmaceutical dosage unit according to said third or fourth aspects of the present disclosure, in the folded configuration said device can be folded into a plurality of pleats via folds, each fold being defined between an adjacent pair of said pleats.

Embodiments of the pharmaceutical dosage unit according to said third or fourth aspects of the present disclosure relate to a pharmaceutical dosage unit for oral administration of a pharmaceutically active cannabinoid or a mixture of at least two pharmaceutically active cannabinoids, comprising a gastro-retentive cannabinoid delivery device folded into a capsule. In some embodiments, for example where said delivery device does not comprise IR layer/s, the said capsule can further contain an emulsion of said at least one cannabinoid or cannabis extract in a pharmaceutically acceptable emulsifying agent.

In a further, fifth aspect, disclosed herein is a gastro-retentive drug delivery dosage form for oral intake, having a first configuration for oral intake and a second configuration for gastric retention, the device comprising a controlled release functional member comprising a drug in an emulsified form. In some embodiments, this gastro-retentive drug delivery device of can further optionally comprise a functional member for immediate release of an emulsified drug which is identical to or different from said drug contained in said controlled release functional member.

The gastro-retentive drug delivery device of this fifth aspect of the present disclosure, that can be ingested when in said first configuration, is configured to assume said second configuration upon exposure to gastric fluids. This device can also be configured for enabling the preservation of said second configuration to provide gastric retention. For example, the device can comprise means for preservation of said second configuration provide gastric retention.

In some embodiments, the gastro-retentive drug delivery device according to this fifth aspect of the present disclosure, said drug is released from said device in a controlled rate of release, or combined controlled rate and immediate rate of release. In specific embodiments, the said drug can be emulsified in a pharmaceutically acceptable emulsifying agent, for example, but not limited to any one of oil, glyceride, water insoluble surfactant, water soluble surfactant or co solvent, or any mixture of at least two thereof. The said emulsified drug is released in emulsified form.

In specific embodiments of the gastro-retentive drug delivery device according to this fifth aspect of the present disclosure, said drug is a drug having log P>2. In some specific embodiments of this fifth aspect, said drug is at least one pharmaceutically active cannabinoid and/or cannabis extract.

The gastro-retentive drug delivery device according to this fifth aspect of the present disclosure, in its said first configuration for oral intake can be contained in a capsule. Said capsule can further contain an emulsion in a pharmaceutically acceptable emulsifying agent of at least one pharmaceutically active drug which is identical to or different from said at least one drug in said controlled release functional member.

In all aspects and embodiments of the present disclosure, the disclosed gastro-retentive drug delivery device or pharmaceutical dosage unit in which the drug is at least one cannabinoid or cannabis extract, can be used in methods for any one of treating, alleviating and preventing worsening of a disease, disorder or condition responsive to cannabinoid therapy in a subject in need, said method comprising orally administering to said patient said gastro-retentive drug delivery device or pharmaceutical dosage unit.

The said disease, disorder or condition responsive to cannabinoid therapy can be any one of anorexia associated with weight loss in patients with AIDS, nausea and vomiting associated with cancer chemotherapy, pain, anxiety, depression, muscle spasticity, arthritis and rheumatism, multiple sclerosis and other neuromuscular inflammatory disorders, inflammatory bowel diseases such as Crohn's disease and colitis, post-traumatic stress disorder (PTSD) or epileptic seizures. Parkinson's disease, spinal cord injury, fibromyalgia, Alzheimer's disease and dementia or any other condition responsive to cannabinoid therapy.

The present disclosure also relates to a method for any one of treating, alleviating and preventing worsening of a disease, disorder or condition responsive to cannabinoid therapy in a subject in need, said method comprising orally administering to said patient a gastro-retentive drug delivery device or pharmaceutical dosage unit according to all aspects and embodiments thereof disclosed herein.

In the disclosed methods, said disease, disorder or condition responsive to cannabinoid therapy can be any one of anorexia associated with weight loss in patients with AIDS, nausea and vomiting associated with cancer chemotherapy, pain, anxiety, depression, muscle spasticity, arthritis and rheumatism, multiple sclerosis and other neuromuscular inflammatory disorders, inflammatory bowel diseases such as Crohn's disease and colitis, post-traumatic stress disorder (PTSD) or epileptic seizures. Parkinson's disease, spinal cord injury, fibromyalgia, Alzheimer's disease and dementia or any other condition responsive to cannabinoid therapy.

The present disclosure also relates to a method for providing a subject in need thereof with stable therapeutically effective plasma level of at least one cannabinoid or mixture of at least two cannabinoids and/or active metabolites thereof over a prolonged period of time and/or increasing the oral absorption time of at least one cannabinoid or mixture of at least two cannabinoids, said method comprising orally administering to said patient a gastro-retentive drug delivery device or a pharmaceutical dosage unit according to all aspects and embodiments thereof disclosed therein.

The present disclosure also relates to a method of increasing the absorption time of an active pharmaceutical ingredient (API) having log P>2 in a subject in need thereof, by administering to said subject a gastro-retentive device or a pharmaceutical dosage unit according to said first, second and thirds aspects and embodiments thereof.

In all aspects and specific embodiments of the presently disclosed subject matter, methods of treatment and therapeutic uses of the presently disclosed pharmaceutical dosage unit can comprise administration of the dosage form once, twice three times a day.

### BRIEF DESCRIPTION OF THE DRAWINGS

In order to better understand the subject matter that is disclosed herein and to exemplify how it can be carried out in practice, embodiments will now be described, by way of non-limiting example only, with reference to the accompanying drawings, in which:

FIG. 1 SEM picture of Film A of Example 1

FIG. 2 Dissolution test of THC+CBD Accordion Pill of Example 1

FIG. 3 Cytation 3 microscope inspection of the dissolution medium of Example 1, at 1 (A) and 8 hours (B) in Cytation 3 microscope

FIG. 4 Dissolution sampling uniformity test of Accordion Pill of Example 1

FIG. 5 SEM picture of Film C of Example 2

FIG. 6 Dissolution test of THC+CBD Accordion Pill of Example 2

FIG. 7 Dissolution sampling uniformity test of Accordion Pill of Example 2

FIG. 8 Cytation 3 microscope inspection of the dissolution medium of Example 2, at 1 (A) and 8 hours (B) in Cytation 3 microscope

FIG. 9 Emulsion stability test of Inner A —CBD

FIG. 10 Emulsion stability test of Inner A—THC

FIG. 11 Emulsion stability test of Inner C—CBD

FIG. 12 Emulsion stability test of Inner C—THC

FIG. 13 Micelles size distribution of Inner Film A and Inner Film C

FIG. 14 Micelles size distribution of various inner films

FIG. 15 Micelles size distribution of various inner films

FIG. 16 CBD (in lipid based solution) dissolution profile

FIG. 17 Vitamin E (Accordion Pill of Example 9) dissolution profile

FIG. 18 Vitamin E (Accordion Pill of Example 9) micelle size distribution

FIG. 19 Vitamin E (Accordion Pill of Example 10) dissolution profile

FIG. 20 Vitamin E (Accordion Pill of Example 10) micelle size distribution

FIG. 21A Fenofibrate (Accordion Pill of Example 11) dissolution profile

FIG. 21B Fenofibrate (Accordion Pill of Example 12) dissolution profile

### DETAILED DESCRIPTION OF EMBODIMENTS

### General

In a first aspect, disclosed herein are oral gastroretentive drug delivery device and oral gastroretentive drug dosage form, for extended release of poorly water-soluble drugs, in which the drug is in an emulsified form. The disclosed delivery devices and dosage forms improve bioavailability of the drug, prolong the absorption phase of the drug, resulting in lower peaks, and lead to stable therapeutically effective and reliable plasma levels for prolonged periods of time. Treatment with the disclosed extended release delivery devices and dosage forms improves both magnitude and duration of the drug pharmacodynamic effects, maximizing therapeutic effects and minimizing any negative side effects. The disclosed delivery devices and dosage forms enable administration of predetermined therapeutic doses of the drug and reduction of the number of daily administrations, leading to better patient's compliance.

In a second aspect, provided herein are gastro-retentive oral formulation, delivery device and dosage form for extended release of cannabinoids. Extended release of cannabinoids can significantly prolong the absorption phase of the drug, resulting in lower peaks and leading to stable therapeutically reliable plasma levels for prolonged period of time. Treatment with the herein disclosed extended release formulations delivery device and dosage forms of cannabinoids can improve both magnitude and duration of cannabinoids pharmacodynamic effects, maximizing therapeutic effects and minimizing any negative side effects. Importantly, such formulation can enable administration of predetermined therapeutic cannabinoid doses, and reduction of the number of daily administrations. Predetermined doses of cannabinoids help prevent overdosing and undesired toxic effects. Extended release cannabinoid formulations can lead to higher compliance of the patient to treatment.

In a further, third aspect, disclosed herein are gastro-retentive drug delivery device and gastro-retentive drug delivery dosage form for oral intake, having a first configuration for oral intake and a second configuration for gastric retention, the device or dosage form comprising a controlled or extended release functional member comprising a drug in an emulsified form.

Disclosed herein are thus oral gastro-retentive pharmaceutical extended delivery devices of emulsified poorly water-soluble drugs. In some aspects and embodiments of the present disclosure, the disclosed gastro-retentive drug delivery device is configured to be folded into or compacted in an orally administered capsule. In such aspects and embodiments, the delivery device is essentially a gastro-retentive multi-layered assembly for delivery of poorly insoluble drugs, including cannabinoids, configured to be folded in a suitable configuration, for example, but not limited to an accordion configuration (which is also referred to herein as “pleated configuration” or “accordion pleated configuration”), and compacted into/packed in a peroral capsule. Delivery devices of such accordion pleated configuration are described in the art [6] The delivery device is, for example, any of the specific delivery devices described herein. The disclosed delivery device can be configured for administration per se, or are comprised in oral pharmaceutical dosage forms.

Disclosed herein are gastro-retentive pharmaceutical dosage forms, which can be dosage unit forms of emulsified poorly water-soluble drugs, for oral administration. In some aspects and embodiments disclosed herein, the pharmaceutical dosage forms comprise a gastro-retentive drug delivery device, in some embodiments a delivery device as disclosed herein, as disclosed herein, folded into or compacted in an orally administered (peroral) capsule. The delivery device can be essentially a gastro-retentive multi-layered assembly for delivery of cannabinoids, folded in a suitable configuration, for example, but not limited to an accordion configuration (which is also referred to herein as “pleated configuration” or “accordion pleated configuration”), and compacted into/packed in a peroral capsule. The delivery device comprises in the disclosed dosage forms is, for example, any of the specific delivery devices described herein.

In all aspects and embodiments disclosed herein, the present disclosure further provides for specific delivery systems for poorly water-soluble drugs, including but not limited to cannabinoids as herein defined, namely orally administered gastroretentive formulations of such drug as disclosed herein, in which the poorly soluble drug is in emulsified form. The disclosed poorly water-soluble drugs formulations can provide stable plasma levels of the drug for longer duration, prolonging drug availability, thereby leading to potential improvements in efficacy of the active principle. The proposed drug delivery systems can generate a continuous and effective exposure of target organs and tissues to the drug, the formulation being gastroretentive, releasing the drug in the stomach for a prolonged time. The disclosed drug delivery system can improve the efficacy of treatment, while reducing the number of daily doses. The disclosed drug delivery system provides for use of a predetermined dose of the active drug. Importantly, the drug delivery device in accordance with all aspects and embodiments of the present disclosure can be versatile in term of the rate of release of the active ingredient. Mainly, while the release is controlled release or extended release, its rate can be controlled by specific features of the delivery device, for example the gastro-retentive multilayered delivery device according to aspects and embodiments of the present disclosure. Such features can be the load of the drug, the features of the drug emulsion, such as the specific emulsion formulation, the API: emulsifying agents ratio, micelles size, load of hydrogel in the various layers, the geometry and structure and structure of the layers, as detailed below. Controlled release can be accompanied by immediate release of portion/s of the dose contained in the disclosed delivery device.

Also disclosed herein are pharmaceutical dosage forms, for example dosage unit forms of cannabinoids. Essentially, a pharmaceutical dosage form as disclosed herein comprises a gastro-retentive cannabinoid/s delivery device, folded into or compacted in an orally administered capsule. In some aspects and embodiments disclosed herein, the cannabinoid/s delivery device is essentially a gastro-retentive multi-layered assembly for delivery of cannabinoids, folded in a suitable configuration, for example, but not limited to an accordion configuration (which is also referred to herein as “pleated configuration” or “accordion pleated configuration”), and compacted into/packed in a peroral capsule. The cannabinoids delivery device is, for example, any of the specific cannabinoid/s delivery devices described herein.

The present disclosure further provides for specific delivery systems for cannabinoids, namely orally administered gastroretentive formulations of cannabinoids as disclosed herein. The disclosed cannabinoid formulations can provide stable plasma levels of the cannabinoid drug for longer duration, prolonging drug availability, thereby leading to potential improvements in efficacy of the active principle. The proposed cannabinoid delivery systems can generate a continuous and effective exposure of target organs and tissues to the drug, the formulation being gastroretentive, releasing the drug in the stomach for a prolonged time. The disclosed cannabinoid delivery system can improve the efficacy of treatment, while reducing the number of daily doses. Moreover, the disclosed cannabinoid delivery system can provide for use of a predetermined dose of the active cannabinoid ingredient/s. Importantly, the drug delivery device in accordance with the present disclosure can be versatile in term of the rate of release of the active ingredient. Mainly, while the release is controlled release, its rate can be controlled by specific features of the multilayered system comprising the gastro-retentive delivery device according to the present disclosure. Such features can be the load of the drug, load of hydrogel in the various layers, their geometry and structure, as detailed below. Controlled release can be accompanied by immediate release of portion/s of the dose contained in the disclosed delivery device.

### Definitions

The terms “drug”, “active substance”, “API” (Active Pharmaceutical Ingredient) or “active principle” or “active ingredient”, used herein interchangeably, refer to a pharmaceutically active substance that provides a therapeutic/physiological effect to a patient, and can also refer to a mixture of at least two thereof.

The terms “cannabinoid”, “cannabinoid derivative” or “cannabinoid metabolite”, as used interchangeably herein, refer to any active principle of cannabis, including but not limited to Δ-9-tetrahydrocannabinol (Δ9-THC, THC), iso-tetrahydrocannabimol (iso-THC), cannabinol (CBN), cannabidiol (CBD) cannabigerol (CBG), cannabichromene (CBC), cannabielsoin (CBE), cannabicyclol (CBL), cannabicitran (CBT), cannabivarin (CBV), tetrahydro-cannabivarin (THCV), cannabidivarin (CBDV), and many others such as tetrahydrocannabidiol (THCBD), tetrahydrocannabigerol (THCBG), tetrahydrocannabichromene (THCBC), tetrahydrocannabidivarol (THCBDV), cannabichromevarin (CBCV), cannabigerovarin (CBGV) and cannabigerol conomethyl ether (CBGM) and pharmaceutically acceptable derivatives thereof, and pharmaceutically active metabolites thereof. The “cannabinoid” as defined herein can also be a cannabimimetic, not derived from cannabis. The terms “cannabinoid”, “cannabinoid derivative” or “cannabinoid metabolite”, as used interchangeably herein, refer also to any mixture of at least two cannabinoids, as defined herein.

The terms “cannabis extract” or “cannabis concentrate” as used interchangeably herein, refer to an extract of the cannabis plant which contains cannabinoids and optionality terpene/s and/or other compounds.

The terms “drug-releasing formulation” and “cannabinoid-releasing formulation” as used herein refer to any composition of matter which comprises a drug, respectfully cannabinoid as herein defined, and releases them upon administration.

The term “poorly water-soluble drug” as used herein refers to a drug which is insoluble or only slightly soluble in water, and specifically refers to “drug having a Log P>2”, which as used herein refers to drugs of which the logarithm of the partition coefficient, defined as a particular ratio of the concentrations of a solute between water and octanol, is >2.

The terms “emulsified drug” or “a drug emulsified in an emulsifying agent” or “a drug emulsion” or “an emulsion of a drug in an emulsifying agent” or “a drug in emulsified form” or the like, used herein interchangeably, refer to poorly water soluble drug/s which is/are dissolved in an emulsifying agent, and upon dilution in aqueous medium such as the gastrointestinal (GI) fluid, can form fine oil in water (o/w) emulsions or microemulsions.

The terms “an emulsifying agent” or “emulsifier” as used herein refer to a compound or substance that concentrates at the interface of two immiscible phases, usually an oil and water. It lowers the interfacial free energy, reduces the interfacial tension between the phases, and forms a film or barrier around the droplets of the immiscible, discontinuous phase as they are formed, preventing the coalescence of the droplets. The term “emulsifying agent” includes, but is not limited to oils, glycerides, water insoluble surfactants, water soluble surfactants or co-solvents or any mixture of at least two thereof.

As used interchangeably herein, “dosage units”, “dosage forms”, “oral dosage units”, “dosage unit forms”, “oral dosage unit forms” and the like refer to solid dosage forms as known in the art. The dosage forms are intended for peroral use, i.e. to be swallowed (ingested) by a patient in need thereof.

The terms “multilayered polymeric assembly”, “drug delivery device” and “accordion”, or similar terms, as used interchangeably herein, refer to a component of the disclosed dosage units, comprising at least one drug. Thus, “cannabinoid delivery device”, for example, refers to any of “multilayered polymeric assembly”, “drug delivery device” or “accordion”, in which the contained drug is at least one cannabinoid, cannabis extract or at least one cannabimimetic, or any mixture of at least two thereof. An “accordion” is usually folded in characteristic undulated/pleated manner and inserted or compacted or packed into a capsule, to provide the dosage unit. The “multilayered polymeric assembly”, or “drug delivery device” or “cannabinoid delivery device” can also be folded in other manners, as described herein.

As used herein, “gastroretentive delivery device”, “gastroretentive drug delivery device”, “gastroretentive cannabinoid delivery device”, “gastro-retentive multilayered polymeric assembly” or “accordion”, used herein interchangeably, refer to gastro-retentive device or system or assembly or the like, carrying or comprising or containing a drug, for example but not limited to a cannabinoid as herein defined, which can be orally administered to a subject in need “as is”, i.e. as a dose unit, or can be comprised within a gastro-retentive dose or dosage unit. All of these gastro-retentive drug delivery devices, systems, etc. generally have a first configuration for oral intake or ingestion, and are capable of assuming a second configuration following ingestion, for gastric retention. A non-limited example is a folded device, for example, an accordion, which unfolds in the stomach upon contact with gastric fluids, and is gastro-retentive as defined herein.

As used herein, “gastroretentive dosage unit(s)”, “gastroretentive dosage form(s)”, and “gastroretentive drug formulation(s)” (GRDF or GRDFs in the plural), or “gastroretentive pharmaceutical composition(s)”, and the like, used interchangeably herein, refer to dosage units and drug formulations with delayed emptying/evacuation from the stomach (also referred to as gastric emptying/evacuation), or longer retention in the stomach, as compared to ingested food. In particular, the terms refer to a gastroretentive folded “accordion” or otherwise folded gastroretentive “multilayered polymeric assembly” or “gastroretentive drug delivery device” or gastroretentive “cannabinoid delivery device” as herein defined, folded or packed or compacted or fitted into a capsule, which unfold upon contact with the gastric fluids, generally after disintegration of the capsule. “Gastric retention” is the retention, maintenance or withholding of a cannabinoid or a component of the dosage unit comprising cannabinoid in the stomach, for a time period longer than the time it would have been retained in the stomach when delivered in a free form or within a gastro-intestinal (GI) delivery vehicle which is not gastroretentive. Gastroretentivity can be characterized by retention in the stomach for a time period that is longer than the normal emptying time from the stomach, i.e. longer than about 2 hours, particularly longer than about 3 hours and usually more than about 4, 5, 6, 7, 8 or 10 hours. Gastroretentivity typically means retention in the stomach for from about 3, 4, 5, 6, 7, 8, 9, 10 or at times 12, 14, 18 hours up to about 24 hours.

“Controlled-release” or “extended release” as used herein denotes a manner that a dosage form or drug delivery device and the like, releases a drug, for example but not limited to a cannabinoid, at a controlled rate over extended designable time intervals, specifically predetermined extended time intervals, at needed quantities to achieve a desired drug, for example but not limited to cannabinoid serum level, and produce a prolonged, sustained or delayed pharmacological effect. The controlled release can include a lag in initiation of the release of the drug.

The phrases “prolonged period/s”, “extended time interval/s” and the like, as used interchangeably herein in connection with controlled release, refer to a period of delivery of at least 80% of the drug dose contained in the dosage unit or drug delivery device that lasts for from several hours following administration to about 12 hours following administration, usually from about 3 hours, and up to any one of 4, 5, 6, 7, 8, 9, 10, 11 or 12 hours, and often between about 3 and about 5 hours, and also between about 4 to about 6 hours and about 4 to about 8 hours, sometimes one or more of between about 3 to about 6 hours, about 4 to about 8 hours, and about 6 to about 10 hours.

The phrase “immediate release” as used herein denotes a manner that a dosage form or drug delivery device releases the drug, for example but not limited to cannabinoid immediately upon exposure to or contact with gastric medium. By “immediately upon exposure to gastric fluid” is to be understood release within up to 2 hours from said exposure or contact. Specifically, immediate release of a drug, for example, but not limited to cannabinoid is within about 30 to about 60 minutes from exposure to or contact with gastric fluid.

“Simulated gastric fluid” (“SGF”) and “Simulated intestinal fluid” (“SIF”) as used herein, refer to “Gastric fluid, Simulated, TS” and “Intestinal fluid, Simulated, TS” solutions as defined by the United States Pharmacopeia 30 National Formulary, without the corresponding enzymes.

“Gastric medium”, “gastric fluid,” and “intestinal medium” as used interchangeably herein denote a biological medium of the stomach and intestines, respectively, or an artificial medium, used to mimic the environment of the stomach or intestines, exemplified but not limited to, “Simulated gastric fluid” (“SGF”) and “Simulated intestinal fluid” (“SIF”).

The term “aqueous medium” as used herein denotes liquid media, based on water, specifically “gastric medium”, “gastric fluid”, “intestinal medium”, as defined above, and distilled water.

The term “biodegradable” or “degradable” as used herein is intended as capable of being biochemically, chemically and/or physically processed, reduced or broken down in the body of a patient, within a time period between several seconds to several days from ingestion of the dosage unit.

The phrase “polymer which is not instantly or readily soluble in gastric fluid” and the like, are used herein to refer to a polymer that will gradually dissolve in the GI tract during its residence therein.

The terms “inert” or “inactive” or “inactive ingredient” or “inert ingredient”, as used interchangeably herein refer to components in the GRDF, to the layers of the GRDF, that do not instantly react with the active ingredient or adversely affect its properties, or cause any biological effect upon administration to a subject when administered in reasonable amounts to said subject. The general examples of these components are described in “The Handbook of Pharmaceutical Excipients”, 4th Edition, by Rowe, Sheskey and Weller, Pharmaceutical press, 2003. Additional exemplary list is Inactive Ingredients Guide of the Food and Drug Administration, USA. The term “inert polymeric film” as used herein refers to non-drug-containing polymeric film.

The term “rapidly”, as used herein, in reference to the disintegration time of a capsule and/or a polymeric film or layer, is to be taken as a time interval between submersion of a capsule/film/layer in an aqueous medium to a significant loss of integrity of the capsule/film/layer, and usually is less than 1 minute, but sometimes less than any one of 2, 3, 4, 5, 6, 7, 8, 9, 10, 11, 12, 13, 14, or 15 minutes. In reference to the unfolding time of a folded multilayered polymeric assembly or folded delivery device, the term encompasses time interval of less than 10 minutes, and sometimes less than 15, 20, 25, or 30 minutes.

The terms “swellable” and “swelling” mean, with respect to a polymer, that the polymer is capable of imbibing fluid and expanding when in contact with fluid present in the environment of use. The environment of use is specifically an aqueous medium. The “swelling” material can be either soluble or insoluble in the medium wherein it swells.

The term “relatively low-molecular weight” in reference to a pharmaceutical inert polymer refers to the grades of the polymer that have lower molecular weight and/or molecular number than other members of the polymer product lines. The term “relatively high-molecular weight” polymers, refers to the upper side of the molecular weight and/or molecular number scale of the available polymer product line.

The terms “film”, “layer” and “membrane”, sometimes preceded by the term “polymeric”, are used interchangeably herein in connection with some or all components of the drug delivery devices, the multi-layered GRDFs and their formulations, their preparation and use as described herein, as context dictates.

When the term “polymeric film” as used in connection with the disclosed drug, specifically but not limited to cannabinoid delivery system, it is to be taken to mean an article of manufacture in the form of a film which comprises at least one polymer, and at least one drug, for example but not limited to at least one cannabinoid as a pharmaceutical active agent. When referring to components of the multi-layered GRDFs which do not themselves comprise drug/s, for example but not limited to cannabinoid/s, the term is to be taken to mean an article of manufacture in the form of a film which comprises at least a polymer. In this connection, all polymers comprising the polymeric film/s disclosed herein are pharmaceutically acceptable, cannabinoid-compatible polymers.

The term “contiguous”, as used, for example, with reference to parts of swelling membranes or immediate-release (IR) layers of the multilayered polymeric assembly, is to be taken to mean located in proximity to, physically adjacent to, with or without actually touching, the neighboring components, such as for example a polymeric carrier and/or the frame member accommodating the same. Contiguous layers can be superimposed one on the other. When used with reference to orifices in swelling membrane/s or to a swelling membrane itself or to IR layers, the term is to be understood similarly, taking into account the three-dimensional structure.

The terms “drug-containing layer” “drug-containing layer” or “drug-containing polymeric carrier” or “drug-containing polymeric support” or “inner layer” or “inner film” or “internal layer” or “internal film” or “cannabinoid-containing layer” or “cannabinoid-containing polymeric carrier” or “cannabinoid-containing polymeric support” as used are to be taken to mean a polymeric film or polymeric layer comprising at least one polymer and at least one drug, respectively a cannabinoid as defined herein. These terms can relate to a core or innermost layer of the drug, for example cannabinoid delivery device and also to IR layers positioned over the swelling membranes of the delivery device.

The terms “emulsified drug-containing layer” “emulsified drug-containing layer” or “emulsified drug-containing polymeric carrier” or “emulsified drug-containing polymeric support” as used herein, where the drug can be a cannabinoid as defined herein, are to be taken to mean a polymeric film or polymeric layer comprising at least one polymer and at least one drug, respectively a cannabinoid, in emulsified form. These terms can relate to a core or innermost layer of the drug (and also referred to as or “inner layer” or “inner film” or “internal layer” or “internal film”), for example cannabinoid delivery device, and also to IR layers positioned over the swelling membranes of the delivery device.

The term “permanently mounted”, as used in reference to a polymeric film or polymeric carrier placed inside (or accommodated within) a frame member of the multilayered polymeric assembly, generally designates placement of the polymeric carrier within an aperture in the boundaries of the frame member, the frame member enclosing, confining, or retaining the polymeric film or polymeric carrier when disposed/sandwiched between two swelling membranes of the multilayered polymeric assembly. The term can further refer to a time interval between placement of the drug-containing polymeric carrier within a receiving-aperture (“cut-out”, or “excision”) of the frame member, and either the essentially complete release of the drug from the multilayered polymeric assembly, or the collapse of the frame member, whichever is earlier. The term “permanently mounted” as used in reference to a drug-containing polymeric film/frame structure or drug-containing polymeric carrier/frame member structure can also generally designate other forms of attaching (or affixing or connecting) the frame member to the drug-containing polymeric carrier.

The term “polymer” as used herein denotes a polymeric material as known in the art and described in greater detail below, but is also sometimes used, in context of more general aspects, to encompass the polymeric composition of one or more polymers, and optionally at least one plasticizer; i.e. the inert materials of polymeric films comprised in the multilayered polymeric assembly, excluding matter dispersed therein.

The terms “orifices”, “holes”, “perforations”, “openings”, “apertures”, “voids” and the like, when used herein with reference to swelling membranes of the multilayered polymeric assembly, interchangeably refer to structural features of the swelling membranes that provide open communication between an inner facing surface and an outer facing surface of the membrane through the membrane. For example, such structural features allow for passage of cannabinoid/s from the polymeric film in the internal layer to an outside of the outer facing surface of the swelling membrane, and thus to an outside of the device. Such holes can be distributed over the swelling membranes in any desired pattern, and thus provide open communication between an outside of the device and the polymeric carrier in the drug-containing, including cannabinoid-containing member via the holes. For example, such holes can be produced from mechanical or otherwise removal of small portions of the swelling membrane, and moreover the holes can optionally be formed with predefined shape and/or dimensions.

The term “polymeric carrier” or “drug-containing polymeric carrier” or “cannabinoid-containing polymeric carrier” or “polymeric support” or “drug-containing polymeric support” or “cannabinoid-containing polymeric support” as used herein is to be taken to mean that part of the drug-containing or cannabinoid-containing layer in which the drug, respectively cannabinoid/s, and optionally other pharmaceutically active agent/s, are comprised. The term “polymeric carrier unit” or “polymeric support unit” or “polymeric carrier” or “polymeric support” or “carrier” or “carrier unit” or “support” or “support unit” are used herein to designate the polymeric film or polymeric carrier or polymeric support, respectively, of a single multilayered assembly for drug delivery, for example cannabinoid delivery, as disclosed herein. In embodiments of the delivery system according to a first aspect of the present disclosure as described in detail below, the polymeric carrier is accommodated in a polymeric frame member of the delivery device, or otherwise attached or affixed thereto. The polymeric carrier and frame structure is generally the innermost layer of the delivery device. Likewise, polymeric support is generally the innermost layer of a delivery device according to a second aspect of the present disclosure, described in detail below, in which there is no frame member in the delivery system. Generally, the drug-containing, for example cannabinoid/s-containing, “polymeric carrier” or “drug-containing polymeric carrier” or “polymeric support” or “drug-containing polymeric support” or “polymeric carrier unit” or “polymeric support unit” or “polymeric carrier” or “polymeric support” or “carrier” or “carrier unit” or “support” or “support unit”, release the drug/s, for example cannabinoid/s, contained therein in a controlled manner. They are thus also referred to as “controlled release layer/s” or “CR layer/s”.

The terms “immediate release layer” or “IR layer” are used herein interchangeably. The terms “immediate release layer unit” or “IR layer unit” are used herein to designate the IR layer of a single multilayered assembly for drug delivery as disclosed herein. Also the IR layer is a drug-containing layer, for example a cannabinoid-containing layer.

The terms “swelling membrane” or “membrane” or “outer film” are used herein interchangeably. The terms “swelling membrane unit” or “membrane unit” or “outer film” unit are used herein to designate one swelling membrane for a single multilayered assembly for drug delivery, for example cannabinoid delivery, as disclosed herein.

The terms “lamination”, “unification”, “amalgamation”, “fusion” and the like as used interchangeably herein, unless the context clearly indicates otherwise, means in reference to two and more polymeric films or sheets, the action of layering, stacking, filing, piling, superimposing at least one polymeric film over at least one additional polymeric film, bringing them into a tight contact, and under suitable conditions of temperature and ambience, applying pressure sufficient to produce a uniform structure wherein boundaries the individual layers are no longer readily distinguishable, or the individual layers are irreversibly bound to the contiguous layers. The lamination is usually between polymeric sheets of similar to identical composition, and similar thickness, although films of significantly different thickness but similar or identical composition may also be laminated. In addition, the lamination may be also performed on a plurality of films of different composition, if at least two of such films are similar or identical in composition and/or thickness, and if in the obtained structure the boundaries of the individual layers are not readily distinguishable or the individual layers are irreversibly bound to contiguous layers. The term “readily distinguishable” should be understood as being within the aptitude of a person skilled in the art to distinguish the individual layers without application of microscopy or spectrometry.

A “patient” or “subject” as referred to herein is an animal who may be administered with the gastro-retentive pharmaceutical dosage units of the presently disclosed subject matter. In general, where the drug is a cannabinoid as herein defined, the “patient” or “subject” is a human, suffering from a medical condition responsive to cannabinoids. In specific aspects and embodiments of the presently disclosed subject matter, the “patient” or “subject” is a human suffering from any one of anorexia associated with weight loss in patients with AIDS, nausea and vomiting associated with cancer chemotherapy, pain, anxiety, depression, muscle spasticity, arthritis and rheumatism, multiple sclerosis and other neuromuscular inflammatory disorders, inflammatory bowel diseases such as Crohn's disease and colitis, post-traumatic stress disorder (PTSD) or epileptic seizures, Parkinson's disease, spinal cord injury, fibromyalgia, Alzheimer's disease and dementia or any other condition responsive to cannabinoid therapy. In additional or alternative aspects and embodiments of the presently disclosed subject matter, the “patient” or “subject” is a human, suffering from any medical condition responsive to cannabinoid/s, as disclosed herein.

The term “stable” plasma or serum levels of a drug, for example but not limited to a cannabinoid as herein defined or active derivative or metabolite thereof is to be taken to mean a therapeutically effective plasma level of the active cannabinoids and/or active metabolites thereof over suitable period of time. Specifically, stable plasma levels can, for example, refer to continuous therapeutically effective levels, steady state levels, and the like.

The term “AUC” as known in the art and used herein refers to the Area Under the Curve (mathematically known as the definite integral) in a plot of drug concentration in blood plasma vs. time. In practice, the drug concentration is measured at certain discrete points in time and the trapezoidal rule is used to estimate AUC. The AUC (from zero to infinity) represents the total drug exposure over time. The term Cₘₐₓ is the maximum (or peak) serum concentration that a drug achieves in a specified compartment or test area of the body after the drug has been administrated. The related pharmacokinetic parameter tₘₐₓ is the time at which the Cₘₐₓ is observed.

The term “treat” and forms thereof such as “treatment” or “treated” as used herein is to be taken to mean, to prevent a symptom (e.g. pain or nausea), or prevent worsening, or arrest, or alleviate, or improve or cure the patient's disease or condition or symptom associated therewith.

The term “ratio” as used herein, unless otherwise indicated, refers to a weight ratio (also designated w/w)

The terms “basic substance” or “alkaline substance” as used interchangeably herein are to be taken to mean any pharmaceutically acceptable substance which can provide for maintaining a higher pH than that of the environment/microenvironment immediately surrounding or in proximity to or in the vicinity of the active agent, for example cannabinoid, when still accommodated within the polymeric carrier or during or soon after it is release therefrom.

The term “suitable” as used herein is to be taken to mean having the properties that enable providing the defined result.

“About” as used herein generally refers to approximate values. When referred to a dose of cannabinoids in milligrams, “about” should be understood as including the range of a value ±15%. When referred to other values, the term should be understood as including the range of a value ±15%, for example ±15%, ±12%, ±10%, ±8%, ±5%, ±2% or ±1%.

As used in the specification and claims, the forms “a”, “an” and “the” include singular as well as plural references unless the context clearly dictates.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

In a first aspect, the present disclosure relates to a gastro-retentive drug delivery device for oral administration of poorly water-soluble drugs, the device being configured for unfolding from a folded configuration for oral intake to an unfolded configuration for gastric retention. In some embodiments, the device disclosed gastro-retentive drug delivery device can be a multilayered assembly, essentially comprising (a) a drug-containing layer comprising a polymeric carrier and at least one emulsified poorly water-soluble drug, the polymeric carrier comprising at least one film-forming polymer; (b) a polymeric frame member configured for imparting mechanical strength to the device sufficient to enable, upon unfolding of the device, the preservation of said unfolded configuration to provide gastric retention, wherein said polymeric frame member accommodates the emulsified drug-containing layer; and (c) one or two polymeric swelling membranes, each covering at least in part one of the two faces of the emulsified drug-containing layer accommodated within said frame member. At least one of the swelling membranes optionally comprising orifices.

In specific embodiments, the emulsified one drug is in the form of an emulsion thereof in a pharmaceutically acceptable emulsifying agent. The emulsifying agent is any of an oil, glyceride, including partial glycerides, water insoluble surfactant, water soluble surfactant or co-solvent or any mixture of at least two thereof.

In specific embodiments, the weight ratio between the film forming polymer and said drug emulsion is from about 1:2 to about 20:1, i.e. the quotient film-forming polymer/emulsion, expressed as a decimal is for example, about 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20.

In specific embodiments, the weight ratio between said at least one pharmaceutically active drug and said emulsifying agent is from about 2:1 to about 1:20, i.e. the quotient drug/emulsifying agent, expressed as a decimal is for example, about 2, 1.75. 1.5, 1.25, 1, 0.75, 0.5, 0.25, 0.2, 0.15, 0.1 or 0.05.

In specific embodiments, the at least one drug is a drug that has log P>2. In some such embodiments, the at least one drug is a pharmaceutically active cannabinoid or a mixture of at least two pharmaceutically active cannabinoids or a pharmaceutically active cannabis extract.

In some embodiments, the disclosed gastro-retentive drug delivery device further optionally comprises at least one emulsified drug-containing polymeric layer for immediate release (IR) of said at least one drug (IR layer) covering at least in part one said swelling membrane, said at least one, in some embodiments two IR layer/s comprising (1) at least one pharmaceutically acceptable film-forming polymer and (2) at least one pharmaceutically active emulsified drug. The said drug-containing IR can each cover at least in part one said swelling membrane.

In some embodiments, in the disclosed gastro-retentive drug delivery device the at least one pharmaceutically active emulsified drug and at least one film-forming polymer are distributed essentially homogeneously throughout the said polymeric carrier, and/or said IR layer/s.

In specific embodiments, the said emulsified mixture of at least two pharmaceutically active cannabinoids comprises THC and CBD at a ratio of from about 20:1 to about 1:20, i.e. the quotient THC/CBD, expressed as a decimal is for example, about 20, 15, 10, 5, 1, 0.75, 0.5, 0.25, 0.1 or 0.05. This mixture can be the same in said polymeric carrier, and/or in said IR layer/s and/or in said additional amount of cannabinoid/s contained in said capsule, or different.

In specific embodiments of the disclosed dosage unit, wherein said drug delivery device comprises IR layer/s, in the disclosed pharmaceutical dosage unit the delivery device comprises a total of from about 1 to about 350 mg of a mixture of THC and CBD, distributed between said polymeric carrier and said at least one IR layer, respectively said additional suitable amount of cannabinoid/s emulsion contained in said capsule, at a ratio of from about 1:10, to about 10:1, wherein the ratio THC:CBD in said mixture comprised in said polymeric carrier and in said at least one IR layer, respectively in said additional suitable amount of cannabinoid/s emulsion contained in said capsule, which can be the same or different is from about 1:20 to about 20:1. The said emulsified cannabinoid or emulsified mixture of at least two cannabinoids comprised in said polymeric carrier or said IR layer/s of said drug delivery device or said cannabinoid emulsion contained in said capsule can be the same or different, in respect of the specific cannabinoids and/or the ratio therebetween in said mixture.

In embodiments of the disclosed gastro-retentive drug delivery device of said first aspect of the presently disclosed subject matter and/or said pharmaceutical dosage form of said second aspect, the said emulsified drug is released from said drug delivery device in emulsified form. The emulsified drug contained in said polymeric carrier is released at a controlled, sustained or extended rate. Release of the drug from said IR layer/s or said additional amount of drug emulsion contained in said capsule is immediate.

In a third aspect, the present disclosure relates to a pharmaceutical dosage unit for oral administration of a pharmaceutically active cannabinoid or a mixture of at least two cannabinoids or a cannabis extract, comprising gastro-retentive cannabinoid delivery device and a capsule, the device being configured for unfolding from a folded configuration for oral intake to an unfolded configuration for gastric retention. In some embodiments, the disclosed gastro-retentive drug delivery device can be a multilayered assembly, essentially comprising (a) a cannabinoid-containing layer comprising a polymeric carrier and at least pharmaceutically active cannabinoid or cannabis extract, the polymeric carrier comprising at least one film-forming polymer; (b) a polymeric frame member configured for imparting mechanical strength to the device sufficient to enable, upon unfolding of the device, the preservation of said unfolded configuration to provide gastric retention, wherein said polymeric frame member accommodates the cannabinoid-containing layer; and (c) one or two polymeric swelling membranes, each covering at least in part one of the two faces of the cannabinoid-containing layer accommodated within said frame member, wherein at least one of the swelling membranes optionally comprising orifices, wherein the device in its folded configuration is contained in said capsule.

In a fourth aspect, provided is a multilayered assembly which comprises a cannabinoid-containing layer, and one or two polymeric swelling membrane/s. When the multilayered assembly comprises two polymeric swelling membranes, the drug-containing layer is sandwiched between them. The drug-containing layer comprises a polymeric support comprising at least one polymer and at least one cannabinoid, and possesses substantial mechanical strength, which provides, at least in part, for gastric retention of the delivery system over a period of time of at least 4 hours. A swelling membrane/s each cover/s one side (face) of the drug-containing layer at least in part, or completely. The various layers of this multilayered assembly are joined together, by suitable means. In embodiments of the second aspect of the present disclosure, the swelling membrane/s is/are optionally perforated. The formed multilayered assembly can be optionally laminated with one or two IR layers, each covering at least in part one of the swelling membrane, respectively. The IR layer/s comprise cannabinoid/s, and provide for immediate release of the cannabinoid/s, as detailed below. The final assembly, whether without or with IR layer/s can then folded and inserted into a peroral capsule.

In the aspects and embodiments of the gastro-retentive drug delivery device disclosed herein, the at least one film-forming polymer in the disclosed gastro-retentive device is any one of a water-soluble or polymer that is partially or completely soluble in both water and organic solvents, or any mixture of at least two thereof. Specific film-forming polymers can be, for example, any one of povidone, copovidone, hydroxypropyl cellulose, polyethylene oxide, amino-methacrylate copolymer NF, hydroxypropyl methylcellulose, hydroxyethyl cellulose, carboxymethyl cellulose or polyvinyl alcohol-polyethylene glycol graft copolymer or any combination of at least two thereof.

In the aspects and embodiments of the gastro-retentive drug delivery device disclosed herein, the polymeric carrier can further optionally comprise at least one pharmaceutically acceptable plasticizer. Specific plasticizers can be, for example, any one of polyethylene glycols, citrate esters, phthalate esters, glyceryl esters, short-chain triglycerides, medium-chain triglycerides, long-chain triglycerides, olive oil, hydrogenated castor oil, triacetin, glyceryl stearate, glyceryl behenate, dibutyl sebacate, aliphatic alcohols, fatty acids, pegylated aliphatic alcohols and pegylated fatty acids, phospholipids, sorbitan derivatives, polysorbates, poloxamers, hydrogenated castor oil derivatives or glycerin, propylene glycol, or any combination of at least two thereof.

In the aspects and embodiments of the gastro-retentive drug delivery device disclosed herein, the polymeric carrier can optionally further comprise at least one pharmaceutically acceptable swelling polymer.

In the aspects and embodiments of the gastro-retentive drug delivery device disclosed herein, the polymeric carrier can further optionally at least one pharmaceutically acceptable swelling polymer. The swelling polymer can be, for example, any one of hydroxypropyl methylcellulose, hydroxypropyl cellulose, hydroxyethyl cellulose, polyethylene oxide, carboxymethyl cellulose, a gum, a protein, and any combination of at least two thereof.

In the aspects and embodiments of the gastro-retentive drug delivery device disclosed herein, for example where the active drug is oxidation-sensitive, more particularly where the active drug is a cannabinoid or cannabis extract, the device can optionally further comprise at least one pharmaceutically acceptable antioxidant agent. for example, but not limited to, any one of BHA, BHT, ascorbic acid, ascorbyl palmitate, tocopherol acetate or a combination of at least any two thereof.

In the aspects and embodiments of the gastro-retentive drug delivery device disclosed herein, the at least one IR layer optionally further comprises at least one of a filler, a surface-active material, a disintegrant, an antioxidant agent, or a combination of any two thereof.

In the aspects and embodiments of the gastro-retentive drug delivery device disclosed herein, the said swelling membranes each comprises at least one polymeric combination of a soluble polymer and a polymer which is not instantly soluble in gastric medium. The said soluble polymer can be, for example, any one of hydroxypropyl cellulose gelatin, hydroxypropylmethyl cellulose, hydroxyethyl cellulose, carboxymethyl cellulose and polyethylene oxide. The said polymer which is not instantly soluble in gastric fluid comprised in said swelling membrane can be, for example, any one of methacrylic acid copolymer NF, cellulose acetate phthalate, hydroxypropyl methylcellulose phthalate, hydroxypropyl methylcellulose acetate succinate, polyvinyl acetate phthalate or any suitable mixture of at least two thereof.

In the aspects and embodiments of the gastro-retentive drug delivery device disclosed herein, the said two IR layers optionally further comprise at least one material that is a plasticizer, a filler, a surface-active material or a disintegrant, or a combination of at least two thereof. The said plasticizer in said IR layers can be, for example, any one of polyethylene glycols, citrate esters, phthalate esters, glyceryl esters, short-chain triglycerides, medium-chain triglycerides, long-chain triglycerides, olive oil, hydrogenated castor oil, triacetin, glyceryl stearate, glyceryl behenate, dibutyl sebacate, aliphatic alcohols, fatty acids, pegylated aliphatic alcohols and pegylated fatty acids, phospholipids, sorbitan derivatives, polysorbates, poloxamers, hydrogenated castor oil derivatives or glycerin, propylene glycol, or any combination of at least two thereof. The said disintegrant in said IR layers can be, for example, any one of microcrystalline cellulose, crospovidone, croscarmellose, starch and its derivatives or polacrilin, or any mixture of at least two thereof.

In the aspects and embodiments of the gastro-retentive drug delivery device disclosed herein, the said polymeric frame member comprises at least one polymer that is not instantly soluble in gastric fluid. The said polymer that it not instantly soluble in gastric fluid comprised in said polymeric frame member can be, for example, a degradable enteric polymer which is substantially insoluble at pH less than 5.5. The said polymer that is not instantly soluble in gastric fluid comprised in said polymeric frame member can be, for example, any one of cellulose acetate phthalate, hydroxypropyl methylcellulose phthalate, hydroxypropyl methylcellulose acetate succinate, polyvinyl acetate phthalate and methacrylic acid copolymer NF, and any suitable mixture of at least two thereof. A specific such polymer is methacrylic acid copolymer NF. The said frame member optionally further comprises a plasticizer. The said plasticizer can be any one of a polyethylene glycol (PEG), or a mixture of two or more polyethylene glycols of different molecular weight, such as, for example, any of PEG 200, PEG 300, PEG 400, PEG 540, PEG 600, PEG 800, PEG 1000, PEG 1450, PEG 1540, PEG 3350, PEG 4000, PEG 4500, PEG 6000, or PEG 8000 or PEG 20000. The said plasticizer optionally further includes a poloxamer, medium-chain triglycerides, glycerin, glyceryl ester, a polysorbate, a sorbitan derivative, citric acid esters, dibutyl sebacate, an aliphatic alcohol, such as cetyl alcohol, a fatty acid, such as stearic acid, propylene glycol or any combination of at least two thereof. Specifically, the plasticizer is a PEG, or a mixture of two or more PEGs with different molecular weights, for example a mixture of PEG 400 and PEG 20,000.

In the aspects and embodiments of the gastro-retentive drug delivery device disclosed herein, the said device can further optionally comprise an anti-adhesion layer. The anti-adhesion layer covers at least one said swelling membrane. In some embodiments of the disclosed gastro-retentive drug delivery device comprising IR layer/s, said anti-adhesion layer covers at least in part said IR layer/s.

In the aspects and embodiments of the gastro-retentive drug delivery device disclosed herein, said orifices are provided on one or both of said swelling membranes. Each said at least one swelling membrane can comprise a suitable number of identical or different orifices of one or more suitable dimensions. The orifices are distributed in each said swelling membrane at suitable distribution pattern and/or suitable shape. For example, orifices can be uniformly distributed over the respective at least one said swelling membranes. In other specific embodiments, the orifices are provided on both said swelling membranes and orifices of one said swelling membrane can be staggered with respect to orifices of the other said swelling membrane. In some specific embodiments, each said swelling membrane can comprise, for example, from 2 to 24, specifically from 8 to 24 of said orifices. Each said orifice can have a diameter or width of, for example, between 0.3 mm and 2.5 mm.

In some embodiments of the disclosed gastro-retentive drug delivery device, the said two swelling membranes can be co-extensive with said drug-containing layer.

In specific embodiments of the disclosed gastro-retentive drug delivery device of said first aspect of the presently disclosed subject matter, the said drug is a pharmaceutically active cannabinoid or a mixture of at least two pharmaceutically active cannabinoids or a pharmaceutically acceptable cannabis extract.

In some embodiments of the disclosed gastro-retentive drug delivery device, in the folded configuration said device is folded into a plurality of pleats via folds, each fold being defined between an adjacent pair of said pleats.

In some embodiments, the disclosed gastro-retentive drug delivery device in its said first configuration for oral intake, can be orally administered as such, as a dosage unit. In some such embodiments the device in its said first configuration can be coated with a suitable coating, for smoother ingestion.

In another aspect, disclosed herein is a pharmaceutical dosage unit comprising a gastro-retentive drug delivery device as defined herein in is all embodiments of said first aspect of the present disclosure, and a capsule, wherein said drug delivery device in its folded configuration is contained within the capsule, which can be, for example, a hard gel capsule.

In some embodiments of the said pharmaceutical dosage unit, for example where said gastro-retentive delivery device does not comprise IR layer/s, said capsule can contain a further suitable amount of an emulsion of said at least one drug in a pharmaceutically acceptable emulsifying agent.

The disclosed pharmaceutical dosage units contain a therapeutically effective amount of said at least one emulsified drug, where the said amount can be contained in said drug delivery device, or distributed between said drug delivery device and said further suitable amount of an emulsion of said drug.

In specific embodiment of the disclosed pharmaceutical dosage unit the emulsified drug is at least one emulsified pharmaceutically active cannabinoid or a pharmaceutically active cannabis extract.

In specific embodiments, in the disclosed pharmaceutical dosage unit the delivery device comprises a total of from about 1 to about 350 mg of said at least one pharmaceutically active cannabinoid or mixture of at least two pharmaceutically active cannabinoids or pharmaceutically active cannabis. The said therapeutically effective amount of at least one emulsified pharmaceutically active cannabinoid or emulsified mixture of at least two pharmaceutically active cannabinoids or pharmaceutically active cannabis extract can be distributed between said polymeric carrier and said at least one IR layer.

In other embodiments, the disclosed pharmaceutical dosage unit comprises a total of from about 1 to about 350 mg of said emulsified pharmaceutically active cannabinoid or emulsified mixture of at least pharmaceutically active two cannabinoids or pharmaceutically active cannabis extract, distributed between said polymeric carrier of said drug delivery device and said additional amount of emulsified cannabinoid/s in contained in said capsule.

Disclosed herein are thus multilayered assemblies comprising drug-carrying layer, which comprises a polymeric carrier and a polymeric frame member, the polymeric carrier comprising the active drug, which in aspects is an emulsified poorly soluble drug, for example an emulsified cannabinoid or emulsified mixture of cannabinoids or cannabis extract, in other aspects is a cannabinoid or mixture of at least two cannabinoids, to form a drug-containing polymeric carrier, the drug-containing polymeric carrier being accommodated within a polymeric frame member to form a carrier/frame structure, which structure is sandwiched between two polymeric swelling membranes. The frame member possesses substantial mechanical strength, which provides, at least in part, for gastric retention of the delivery device, also referred to a delivery system over a period of time of at least 4 hours following ingestion. In embodiments of the present disclosure, the swelling membranes are optionally perforated. The swelling membranes cover the carrier/frame structure at least in part, or completely. The various layers of this multilayered assembly are joined together. The resulting multilayered assembly can be optionally laminated with one or two IR layers, which cover at least in part one or both of the swelling membranes, respectively. According to said first and second aspects of the present disclosure, the IR layer/s comprise an emulsified poorly soluble drug, for example an emulsified cannabinoid or a mixture of at least two emulsified cannabinoids or cannabis extract, or according to said third and fourth aspects of the present disclosure a cannabinoid or mixture of at least two cannabinoids or cannabis extract, and provide for immediate release of the emulsified poorly soluble drug, respectively the cannabinoid/s, as detailed below. The final assembly, whether without or with IR layer/s can then be folded and inserted/compacted into an orally administered (peroral) capsule.

As mentioned, generally, the cannabinoid/s-containing polymeric carrier in said third aspect of the present disclosure or cannabinoid/s-containing polymeric support in said fourth aspect of the present disclosure release the cannabinoid/s contained therein in a controlled or sustained manner.

In at least some embodiments of all aspects of the present disclosure, the active emulsified drug, for example emulsified cannabinoid/s or cannabis extract in said first and second aspects, and cannabinoid/s or cannabis extract in said third and fourth aspects, and polymer/polymer mixture comprising the polymeric carrier, or the polymeric support, are distributed essentially homogeneously throughout the said polymeric carrier or, where present, polymeric support of the delivery assembly, and, where present, throughout the IR layer/s. As mentioned, according to at least some aspects of the presently disclosed subject matter, the said delivery assembly can be folded and fitted into a gelatin capsule.

The disclosed dosage units comprise as active ingredient at least one emulsified poorly water soluble drug, for example a cannabinoid in said first and second aspects, respectively at least one pharmaceutically active cannabinoid in said third and fourth aspects of the present disclosure. The active agent is comprised in the polymeric carrier layer or polymeric support layer, where present, and where present, in the IR layers. The cannabinoids in the IR layer/s, where present, can be identical with or different from the cannabinoid/s in said polymeric carrier layer or polymeric support layer. The cannabinoid is any one of, but not limited to, Δ-9-tetrahydrocannabinol (Δ9-THC, THC), iso-tetrahydrocannabimol (iso-THC), cannabinol (CBN), cannabidiol (CBD) cannabigerol (CBG), cannabichromene (CBC), cannabielsoin (CBE), cannabicyclol (CBL), cannabicitran (CBT), cannabivarin (CBV), tetrahydro-cannabivarin (THCV), cannabidivarin (CBDV), and many others such as tetrahydrocannabidiol (THCBD), tetrahydrocannabigerol (THCBG), tetrahydrocannabichromene (THCBC), tetrahydrocannabidivarol (THCBDV), cannabichromevarin (CBCV), cannabigerovarin (CBGV) and cannabigerol conomethyl ether (CBGM) and pharmaceutically acceptable derivatives thereof, and pharmaceutically active metabolites thereof pharmaceutically acceptable derivatives thereof, and pharmaceutically active metabolites thereof. The active cannabinoid/s can be comprised in the drug-containing layer together with an emulsifying agent, facilitating release of the cannabinoid. Suitable emulsifying agents are, but not limited to pharmaceutically acceptable oils, like, for example, hydrogenated castor oils, or nonionic surfactants, like polysorbate, or anionic surfactants, like SLS, or polymeric solubilizers, like polyvinyl caprolactam-polyvinyl acetate-polyethylene glycol graft copolymer (Soluplus®). Mixtures of cannabinoids can be used, for example a mixture of THC, which is a psychoactive cannabinoid, and CBD, which is not psychoactive, to provide a synergistic effect. Where mixtures of cannabinoids are used, they can be present at any suitable ratio. For example, where THC and CBD are used, the weight ratio can be from about 1:20 to about 20:1, Using specific ratios of the active cannabinoids, can allow for dose adjustment. The active agent is released by controlled release, for example from the polymeric carrier or polymeric support, and/or immediate release, from the IR layer/s.

Thus, in certain embodiments of all aspects of the presently disclosed delivery device and pharmaceutical dosage units, the emulsified poorly soluble drug, for example cannabinoid/s, respectively the cannabinoid-containing layer can comprise different emulsified drug, respectively cannabinoids, in admixture.

Alternatively, in all aspects of the present disclosure, the said polymeric carrier or, respectively, polymeric support in the cannabinoid-containing layer can each comprise two or more distinct laminated polymeric films, each comprising a different specific cannabinoid or mixture of cannabinoids. In such embodiments of both aspects of the present disclosure, the laminated polymeric films comprising the polymeric carrier, respectively the polymeric support, can optionally be separated one from the other by an inert separating polymeric film. Such inert separating film prevents diffusion or leakage of the cannabinoid or mixture of cannabinoids from one polymeric film to another. The polymer/s constituting the distinct polymeric films can be identical or different. In these embodiments, the two or more laminated polymeric films comprising the polymeric carrier, respectively the polymeric support can each release the active cannabinoid/s at different rates, depending on the features of the cannabinoid and the polymeric film. This enables fine tuning and control of the rates of release.

Still alternatively, the said polymeric carrier or, respectively, polymeric support in the cannabinoid-containing layer can each comprise two or more distinct laminated polymeric films, which are different one from the other in terms of the constituting polymers, each comprising the same specific cannabinoid or mixture of cannabinoids, however, each providing a different rate of controlled release of the cannabinoid/s. also in these embodiments the two distinct polymeric carriers, respectively, polymeric supports, can be separate one from the other by an inert non-drug-containing polymeric film.

Specific, non-limiting combinations of polymers used for the polymeric carrier or polymeric support, for the frame member, where present, for the swelling membranes and the IR layers, are disclosed in the following examples.

The disclosed gastro-retentive delivery devices and/or dosage units comprising a therapeutically effective amount of the active ingredient, for example, at least one pharmaceutically active cannabinoid, in the polymeric carrier, respectively the polymeric support, or the IR layer/s. The effective amount can also be determined in accordance with the desired frequency of administration, which is specifically once, twice daily or three times a day. Where administration is twice daily, administrations can be 12 hours, or more than 12 hours or less than 12 hours apart.

In embodiments of aspects of the present disclosure, the polymeric carrier, respectively the polymeric support, can further optionally comprise a pharmaceutically acceptable alkaline (basic) substance, for example a metal hydroxide or salt or an alkaline buffer, to protect cannabinoids that are susceptible to acid degradation.

The herein disclosed gastro-retentive drug delivery devices or pharmaceutical dosage units comprising the same, can be used in medicine, for example in methods for any one of treating, alleviating and preventing worsening of a disease, disorder or condition responsive to the active drug comprised therein, the methods comprising orally administering to a subject in need of such therapy a gastro-retentive the said drug delivery device or pharmaceutical dosage unit.

Thus, a gastro-retentive drug delivery device or pharmaceutical dosage unit comprising cannabinoid/s, as disclosed in all aspects, can be used for treating, alleviating and preventing worsening of a disease, disorder or condition responsive cannabinoid therapy, by being orally administered to a subject or patient in need. The disease, disorder or condition responsive to cannabinoid therapy can be any of anorexia associated with weight loss in patients with AIDS, nausea and vomiting associated with cancer chemotherapy, pain, anxiety, depression, muscle spasticity, arthritis and rheumatism, multiple sclerosis and other neuromuscular inflammatory disorders, inflammatory bowel diseases such as Crohn's disease and colitis, post-traumatic stress disorder (PTSD) or epileptic seizures, Parkinson's disease, spinal cord injury, fibromyalgia, Alzheimer's disease and dementia or any other condition responsive to cannabinoid therapy. Said administration can be once or twice daily or three times a day, or said administration can be chronic.

Thus, methods for any one of treating, alleviating and preventing worsening of any disease, disorder or condition responsive to cannabinoid therapy in a subject in need, for example any of the said specific disorders, said methods comprising orally administering to said patient a gastro-retentive drug delivery device or pharmaceutical dosage unit as disclosed herein are also encompassed by the present disclosure.

The present disclosure also relates to a method for providing a subject in need thereof with stable therapeutically effective plasma level of at least one cannabinoid or mixture of at least two cannabinoids and/or active metabolites thereof over a prolonged period of time, the method comprising orally administering to said patient a gastro-retentive drug delivery device or a pharmaceutical dosage unit as disclosed herein.

Further, the present disclosure relates to a method of increasing the oral absorption time of a cannabinoid in a subject in need thereof, by administering to said subject a cannabinoid gastro-retentive device or a pharmaceutical dosage unit as disclosed herein.

Furthermore, the present disclosure also relates to a method of increasing the absorption time of an active pharmaceutical ingredient (API) having log P>2, in a subject in need thereof, by administering to said subject a gastro-retentive device or a pharmaceutical dosage unit in which said drug is emulsified, as disclosed in said first and second aspects herein.

Furthermore, the present disclosure also relates to a method of increasing the absorption time of cannabinoid/s or cannabis extract in a subject in need thereof, by administering to said subject a gastro-retentive device or a pharmaceutical dosage as disclosed in all aspects herein.

In addition, the present disclosure relates to a method for providing patient in need with prolonged gastric retention time of at least one cannabinoid or a mixture of at least two cannabinoids, by orally administering to the patient a cannabinoid gastro-retentive device or pharmaceutical dosage unit disclosed herein. The prolonged gastric retention time can be greater than about 4, 5, 6, 7, 8, 9, 10, 11, 12, 13 or 14 hours following said oral administration.

In a fifth aspect, the present disclosure relates to a gastro-retentive drug delivery device for oral intake, having a first configuration for oral intake and a second configuration for gastric retention, the device comprising a controlled release functional member comprising a drug, specifically poorly water soluble drug, which can be in an emulsified form. This gastro-retentive drug delivery device further optionally comprises a functional member for immediate release of a drug, which can be an emulsified drug, which is identical to or different from said drug contained in said controlled release functional member. Said gastro-retentive drug delivery device, ingested when in said first configuration, is configured to assume said second configuration upon exposure to gastric fluids. The gastro-retentive drug delivery device is configured for enabling the preservation of said second configuration to provide gastric retention, and comprises means for preservation of said second configuration, thereby providing for gastric retention. Said drug is released from said gastro-retentive drug delivery device in a controlled rate of release, or combined controlled rate and immediate rate of release. The said drug is emulsified in a pharmaceutically acceptable emulsifying agent, which can be at least one oil, glyceride, water insoluble surfactant, water soluble surfactant or co solvent, or any mixture of at least two thereof. The said emulsified drug is released in emulsified form. Specific drugs can be poorly water soluble drugs, for example drugs having log P>2. For example, the said drug can be at least one pharmaceutically active cannabinoid and/or cannabis extract. The gastro-retentive drug delivery device according to this fifth aspect of the present disclosure, in its said first configuration for oral intake can be contained in a capsule, for example a hard gel capsule. The capsule containing the said drug delivery device can also be referred to herein as a dosage unit of poorly soluble drug. The capsule containing the gastro-retentive poorly soluble drug delivery device in its said first configuration can further contain an emulsion in a pharmaceutically acceptable emulsifying agent of at least one pharmaceutically active poorly water soluble drug, which can be identical to or different from said at least one drug in said controlled release functional member.

In some specific embodiments of the fifth aspect of the present disclosure, The gastro-retentive device can be, for example, a low density form of the dosage form, that causes buoyancy in gastric fluid; high density dosage form, that is retained in the bottom of the stomach; a dosage form that bioadhesive to stomach mucosa; dosage forms with means for slowing motility of gastrointestinal tract, for example by concomitant administration of suitable drugs or pharmaceutical excipients; or dosage forms capable of expansion by swelling or unfolding to a large size, thereby capable of gastric retention.

The gastro-retentive drug delivery device of the fifth aspect of the present disclosure can be used therapeutically, for example in all of the methods described above.

The presently disclosed subject matter is further illustrated by the following examples, which are illustrative only and are not to be construed as limiting the scope of the invention. Variations and equivalents of these examples will be apparent to those skilled in the art in light of the present disclosure, the drawings and the claims herein.

It is appreciated that certain features of the presently disclosed subject matter which are, for clarity, described in the context of separate embodiments, can also be provided in combination in a single embodiment. Conversely, various features of the presently disclosed subject matter, which are, for brevity, described in the context of a single embodiment, can also be provided separately or in any suitable sub-combination.

Although the presently disclosed subject matter has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent and patent application was specifically and individually indicated to be incorporated herein by reference. In addition, citation or identification of any reference in this application shall not be construed as an admission that such reference is available as relevant prior art to the presently disclosed subject matter.

### DESCRIPTION OF NON-LIMITING EXAMPLES

### Delivery Devices and Dosage Units with Emulsified Drup

### Example 1: AP-THC 15 mg & CBD 15 mg Formulation 1

### Inner Film:

An exemplary preparation of an emulsion containing THC and CBD and the preparation of a dry film containing THC and CBD micelles, is presented below (referred to throughout the Examples below also as “inner film”, “inner layer” or “inner film unit” or “inner layer unit”). The composition of a single inner unit is summarized in Table 1.

TABLE 1

| Inner film A | mg per inner unit |
|---|---|
| THC | 10 |
| CBD | 10 |
| Labrasol | 40 |
| Kolliphor EL | 40 |
| Klucel EF | 158.8 |
| Klucel GF | 11.2 |
| PEG 400 | 10 |

THC and CBD were dissolved in Labrasol and Kolliphor mixture using magnetic stirrer to obtain a clear solution.

In a 1 Liter mixer, heated to 60° C., PEG 400 was dissolved in water.

The THC and CBD clear solution was added to form a self-emulsion. Klucel EF and Klucel GF were added, and dispersed for about 30 minutes in the heated water. The emulsion was chilled to 30° C. applying low mixing speed until all the Klucel® was dissolved.

The final emulsion was cast on a silicon-coated PET (Mylar™) web, using a table top casting machine with a knife space of 1000-1200 μm. The cast emulsion was dried in an oven at 60° C. for about 120 minutes. The dried product is a film with a solvent content (based on a loss-on-drying test) value of not more that 6%, and a thickness of about 150 μm.

3 sheets of the dry film were stacked one on top of the other and laminated through a roller. The laminated inner films have a thickness of about 450 μm and a weight per area of about 48.6 mg/cm².

The laminated inner film was punched into 17×39 mm octagon units, each comprising 10 mg THC and 10 mg CBD.

Sem Picture:

A sample of Inner Film A was inspected in Scanning Electron Microscope according to the following procedure:

SEM images of Au/Pd coated layer (about 10-20 nm thickness) cross-section of the films were taken using ESEM FEI Quanta 200 system with acceleration voltage of 15 kV. Images were taken using secondary emitted electrons (SE) and backscattered electrons (BSE) detectors.

The SEM picture of Inner Film A is presented in FIG. 1.

The SEM picture confirm that THC and the CBD were in emulsion form in the dry film.

Swelling Membrane (Outer Film):

An exemplary preparation of a solution and the preparation of an outer film are shown below. The composition of two outer films is summarized in Table 2:

TABLE 2

| Outer film | mg per 2 outer films |
|---|---|
| Eudragit S 100 | 23.2 |
| Eudragit L 100-55 | 9.3 |
| Klucel EF | 13.9 |
| Klucel GF | 27.8 |
| PEG 400 | 13.9 |
| Kolliphor 407 | 4.6 |

In a 20 Liter mixer, heated to 50° C., PEG 400 and Kolliphor 407 were dissolved in IPA (isopropanol), for 20 minutes. Klucel GF, Klucel EF, Eudragit S 100 and Eudragit L 100-55 were added, and dissolved for about 120 minutes.

The solution was cast on a silicon-coated PET (Mylar™), using a Web Coater. The dried product is a film with a loss-on-drying value of not more that 7.5%, and a weight per area of 13.6 g/m².

Four sheets of the dry film were laminated together through a roller. The resulting laminate (the outer film) has a thickness of about 45 μm and a weight of 54.4 g/m². The outer films were cut to sheets which cover 16 inner units, and 14 0.5 mm diameter holes per each outer film were perforated.

Frame Film:

An exemplary preparation of a solution and the preparation of the frame film are shown below. The composition a single frame film unit are summarized in Table 3:

TABLE 3

| Frame | mg per frame unit |
|---|---|
| Eudragit L100 | 103.7 |
| PEG 20,000 | 52 |
| PEG 400 | 17.3 |

In a 20-Lilter mixer, heated to 50° C., PEG 400 and PEG 20,000 were dissolved in ethanol, for 60 minutes. Eudragit L100 is added, and dissolved for about 35 minutes. The solution was cast on silicon-coated PET (Mylar™), using a Web Coater.

The dried product is a film with a loss-on-drying value of 6.5-8.5% and a weight per area of 56.0 g/m².

8 sheets of the dry film were laminated together through a roller.

The thickness of the 8 sheets laminated film (the frame layer) is about 360 micrometers and a weight of 448.0 g/m².

The laminated frame films were cut to achieve sheets with 16 internal-layer-receiving-apertures of 17×39 mm for the inner units to be inserted.

Supra-Outer Film Layer:

An exemplary preparation of an emulsion containing THC and CBD and the preparation of a supra-outer film containing THC and CBD micelles, is presented below. The composition of two supra-outer units is summarized in Table 4.

TABLE 4

| Supra-outer film B | mg per 2 supra-outer units |
|---|---|
| THC | 5 |
| CBD | 5 |
| Labrasol | 20 |
| Kolliphor EL | 20 |
| Klucel EF | 110 |
| Compracel | 50 |

THC and CBD were dissolved in a Labrasol and Kolliphor mixture using magnetic stirrer to obtain a clear solution.

In a 1 Liter mixer, heated to 60° C., PEG 400 was dissolved in water.

The THC and CBD clear solution was added to form self-emulsion. Klucel EF and Compracel were added, and dispersed for about 30 minutes in the hot water.

The emulsion was chilled to 30° C. applying low mixing speed until all the Klucel was dissolved.

The final emulsion was cast on a silicon-coated PET (Mylar™) web, using a table top casting machine with a knife space of 600 μm. The cast emulsion was dried in an oven at 60° C. for about 60 minutes. The dried product is a film with a solvent content (based on a loss-on-drying test) value of not more that 7%, a thickness of about 150 μm, and weight per area of 10.7 mg/cm²

The supra-outer film was cut into 24×45 mm octagon units, each comprising 2.5 mg THC and 2.5 mg CBD.

Assembly Process:

The different layers were stacked and assembled together in the following order: first supra outer unit, then the first perforated outer film unit, then the frame member, the inner layer unit, a second perforated outer film unit and then the second supra outer unit.

The final laminates were folded in an accordion-like configuration using a folding apparatus, and after folding were inserted into a gelatin capsule.

Dissolution Profile:

The dissolution of the capsules containing the assembled units were tested using the following method:

Dissolution Parameters:

Medium: Simulated Gastric Fluid (SGF), pH 1.2

Temperature: 37±0.5° C.

Apparatus: Enlarged baskets at 100 RPM

Volume: 600 ml

Sampling time points: 0.25; 0.5; 1; 2; 3; 4; 6; 8; 10 hours

In each sampling point, aliquots of 5 ml of the solution were sampled from the vessel. The samples were tested using HPLC method to analyze the THC and CBD content. Results are presented below.

HPLC Parameters:

| Instrument | HPLC with an auto-injector | |
|---|---|---|
| Column | Gemini NX, C18, 5μ, | |
| | (50 × 4.6) mm (or equivalent) | |
| Column temperature | 30° | C. |
| Sample temperature | 5° | C. |
| Mobile phase | Purified Water:Acetonitrile (35:65) | |
| Flow rate | 2.0 | mL/min |
| Run time | Not less than 6.5 minutes | |
| Detection | UV at 210 nm | |
| Injection volume | 20 | mL |

| | |
|---|---|
| Diluent | SGF:Ethanol (1:1) |
| Needle wash | Methanol |

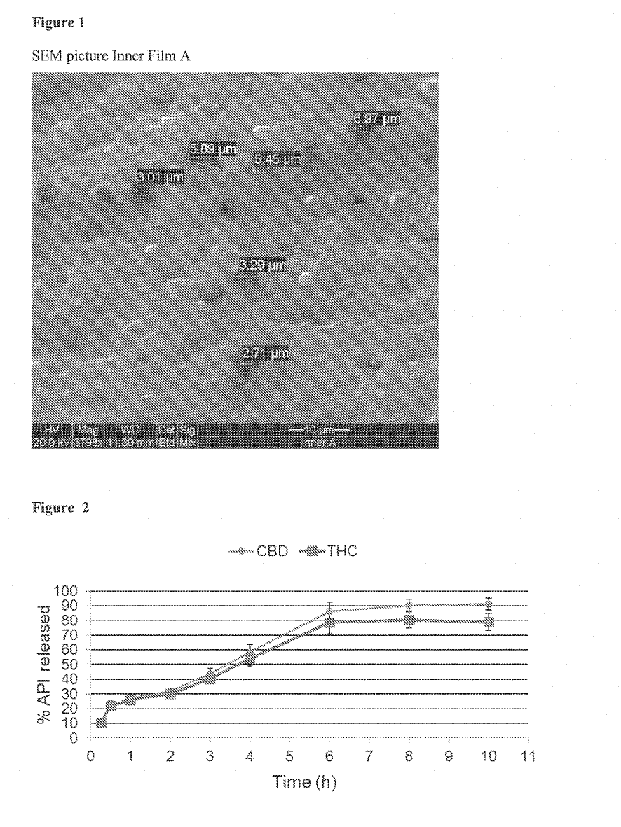

The results of the dissolution test of the Accordion Pill of Example 1 are presented in FIG. 2.

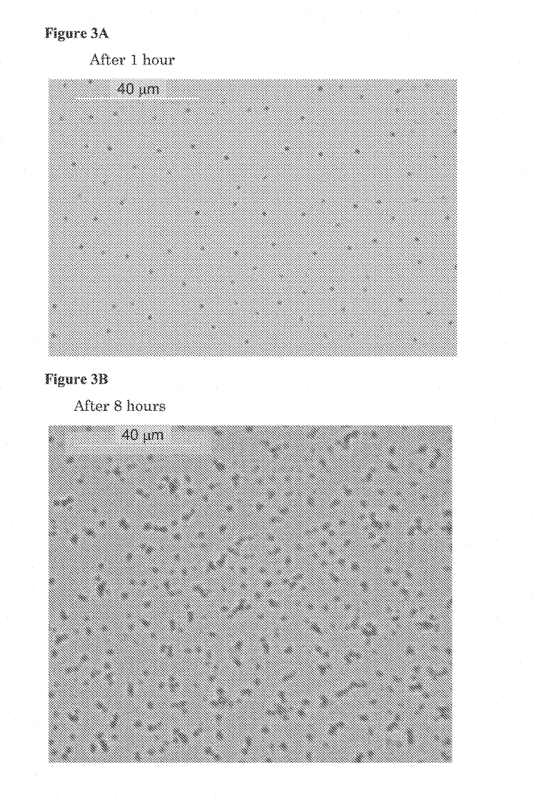

The dissolution medium was inspected after 1 and 8 hours in Cytation 3 microscope, as presented in FIGS. 3A and 3B, respectively.

In addition, a dissolution sampling uniformity test was performed, according to the following method:

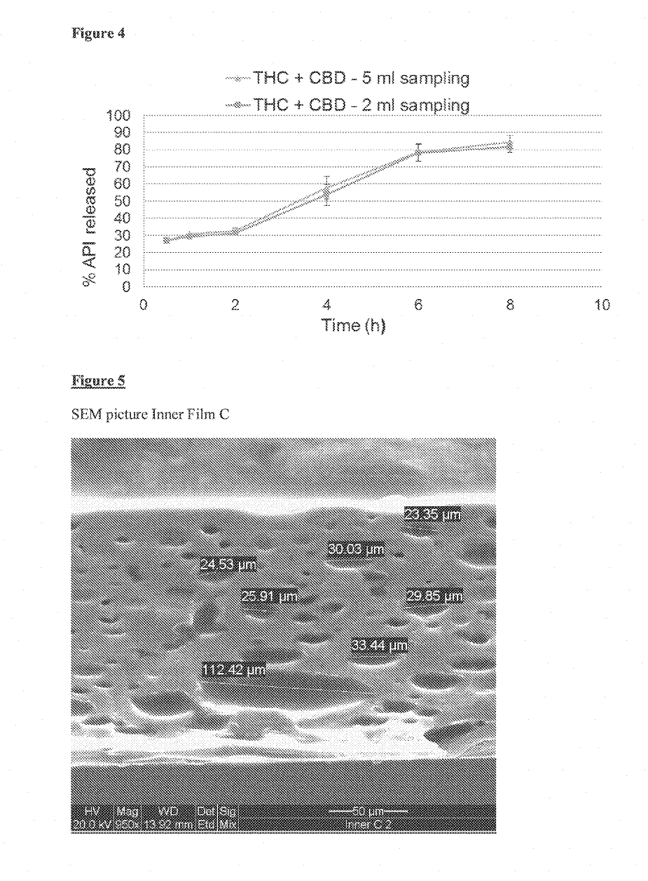

Samples of 2 ml and 5 ml were taken at each dissolution sampling point. The samples were tested using HPLC method to analyze the CBD and THC content. The results of the tests, as described above, are presented in FIG. 4.

Both the 2 ml and 5 ml samples show the same dissolution profile of THC and CBD, showing that the content of the dissolution vessel is homogeneous and the THC and CBD are released as emulsified drugs (otherwise THC and CBD, which are non-soluble in aqueous media, would have precipitated).

### Example 2: AP-THC 15 mg & CBD 15 mg Formulation 2

Inner Film:

An exemplary preparation of an emulsion containing THC and CBD and the preparation of a film containing THC and CBD micelles, is presented below. The composition of a single inner unit is summarized in Table 5.

TABLE 5

| Inner film C | mg per inner unit |
|---|---|
| THC | 10 |
| CBD | 10 |
| Peceol | 24 |
| Kolliphor EL | 56 |
| Klucel EF | 163.5 |
| Klucel GF | 6.5 |
| PEG 400 | 10 |

THC and CBD were dissolved in a Peceol and Kolliphor mixture using magnetic stirrer to obtain a clear solution.

In a 1 Liter mixer, heated to 60° C., PEG 400 was dissolved in water.

The THC and CBD clear solution was added to form a self-emulsion. Klucel EF and Klucel GF were added, and dispersed for about 30 minutes in the hot water.

The emulsion was chilled to 30° C. applying low mixing speed until all the Klucel was dissolved.

The final emulsion was cast on a silicon-coated PET (Mylar™) web, using a table top casting machine with a knife space of 1000-1200 μm. The cast emulsion was dried in an oven at 60° C. for about 120 minutes. The dried product is a film with a solvent content (based on a loss-on-drying test) value of not more that 6%, and a thickness of about 150 μm.

3 sheets of the dry film were stacked one on top of the other and laminated through a roller. The laminated inner films has a thickness of about 450 μm and a weight per area of about 48.6 mg/cm².

The laminated inner film was punched into 17×39 mm octagon units, each comprising 10 mg THC and 10 mg CBD.

SEM Picture

A sample of Inner Film C was inspected in Scanning Electron Microscope according to the following procedure:

SEM images of Au/Pd coated layer (about 10-20 nm thickness) cross-section of the films were taken using ESEM FEI Quanta 200 system with acceleration voltage of 15 kV. Images were taken using secondary emitted electrons (SE) and backscattered electrons (BSE) detectors.

The SEM picture of Inner Film C is presented in FIG. 5.

The emulsion droplets size in Inner C is larger than the emulsion droplets size in Inner A (FIG. 1).

Supra-Outer Film:

An exemplary preparation of an emulsion containing THC and CBD and the preparation of a supra-outer film containing THC and CBD micelles, is presented below. The composition of two supra outer film units is summarized in Table 6.

TABLE 6

| Supra-outer film D | mg per 2 supra-outer units |
|---|---|
| THC | 5 |
| CBD | 5 |
| Peceol | 12 |
| Kolliphor EL | 28 |
| Klucel EF | 110 |
| Compracel | 50 |

THC and CBD were dissolved in a Peceol and Kolliphor mixture using magnetic stirrer to obtain a clear solution.

In a 1 Liter mixer, heated to 60° C., PEG 400 was dissolved in water.

The THC and CBD clear solution was added to form self-emulsion. Klucel EF and Compracel were added, and dispersed for about 30 minutes in the hot water.

The emulsion was chilled to 30° C. applying low mixing speed until all the Klucel was dissolved.

The final emulsion was cast on a silicon-coated PET (Mylar™) web, using a table top casting machine with a knife space of 600 μm. The cast emulsion was dried in an oven at 60° C. for about 60 minutes. The dried product is a film with a solvent content (based on a loss-on-drying test) value of not more that 7%, a thickness of about 150 μm, and weight per area of 10.7 mg/cm²

The supra outer film was cut into 24×45 mm octagon units, each comprising 2.5 mg THC and 2.5 mg CBD.

Outer, Frame, and Assembly Process:

The outer layers, the frame layer, the assembly process and the encapsulation were as described in Example 1.

Dissolution Profile:

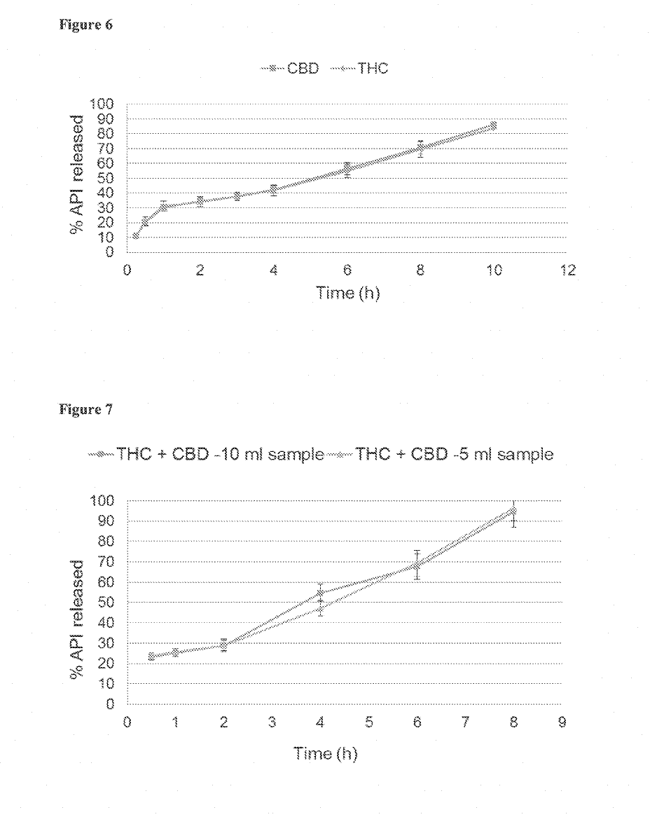

The dissolution of the capsules containing assembled units was tested as described in Example 1. In this case the dissolution medium was phosphate buffer pH=4.5 The results of the dissolution test of the accordion pill of Example 2 are presented in FIG. 6.

In addition, a dissolution sampling uniformity test was performed, according to the following method:

Sample of 5 ml and 10 ml were taken in each dissolution sampling point. The samples were tested using HPLC method to analyze the CBD and THC content.

The results of the test with 2 sampling procedures are presented in FIG. 7.

Both the 5 ml and 10 ml samples show the same dissolution profile of THC and CBD. Thus the content of the dissolution vessel is homogeneous and the THC and CBD are released as emulsified drugs otherwise THC and CBD, which are non-soluble in aqueous media, would have precipitated.

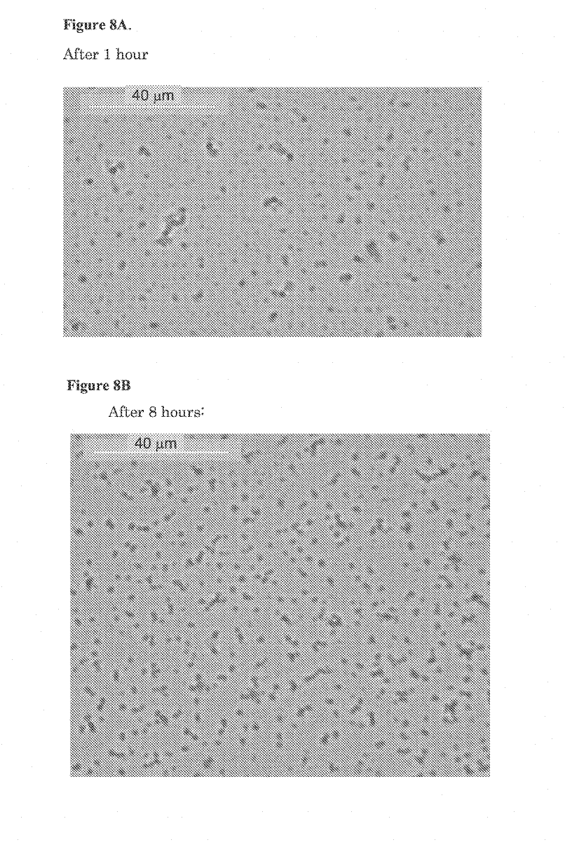

The dissolution medium was inspected after 1 and 8 hours in Cytation 3 microscope. The microscope pictures of the emulsion are presented in FIGS. 8A and 8B, respectively.

### Example 3: Emulsion Stability Test

The stability of the emulsion containing THC and CBD was evaluated, according to the following procedure:

280 mg of inner film A of Example 1, and 280 mg of inner film C of Example 2 were each dissolved in 500 ml SGF, using paddle at 200 RPM for 60 minutes to obtain a cloudy emulsion.

The paddle rotation was stopped and 5 ml emulsion were sampled from the vessel at time 0, 8 and 24 hours. The samples were tested using HPLC method to analyze the CBD and THC content, as described in Example 1.

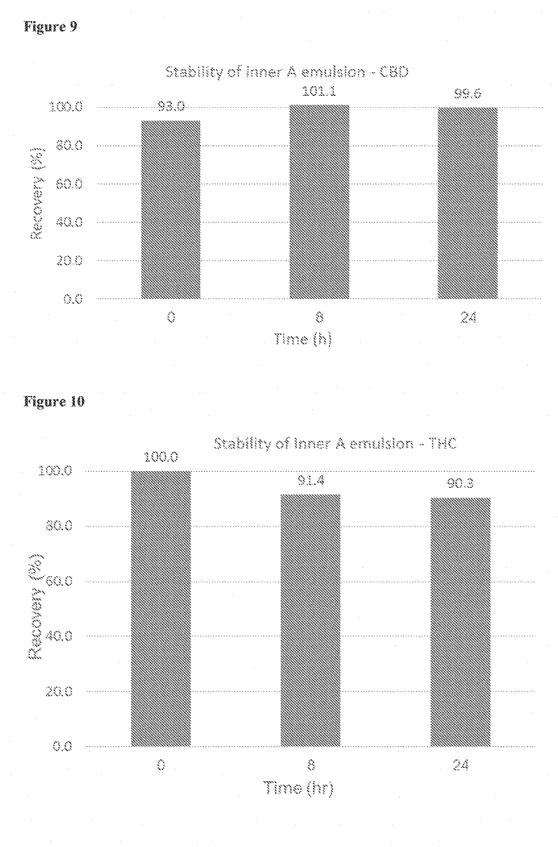

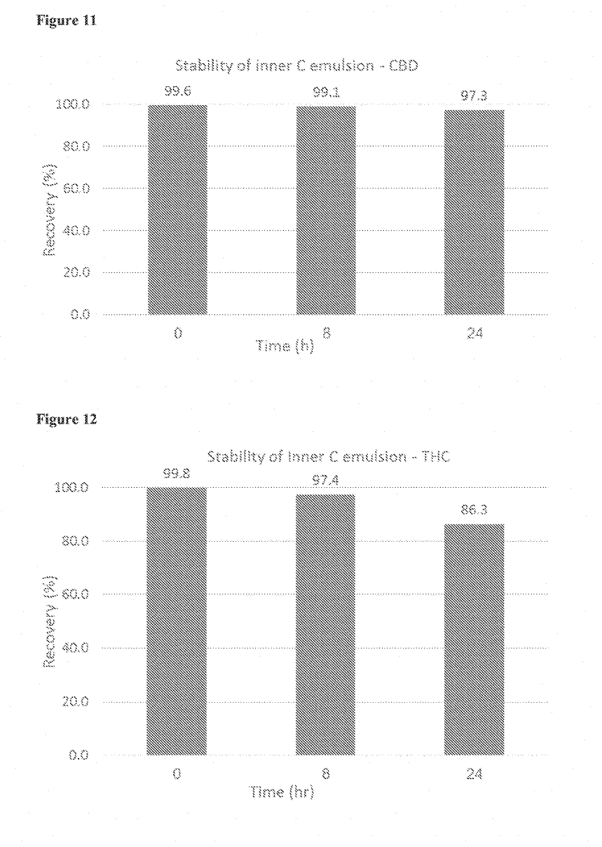

The results of emulsion stability test are presented in FIGS. 9-12.

The results show that the emulsion was stable for at least up to 24 h (the slight decrease of the THC assay is due to degradation of the molecule and not due to precipitation).

### Example 4: Emulsion Homogeneity Test

The homogeneity of the emulsion containing THC and CBD after dissolving an inner film was tested using the following method:

280 mg of inner film A of Example 1, and 280 mg of inner film C of Example 2 were each dissolved in 500 ml SGF, using paddle at 200 RPM for 60 minutes to achieve cloudy emulsion.

The paddle rotation was stopped and 5 ml of the emulsion were sampled from the top, bottom and mid-height of the dissolution vessel.

The samples were tested using HPLC method to analyze the THC and CBD content as described above.

The results of micelles homogeneity test are presented in Table 7.

TABLE 7

| Inner Film | Compound | Place | Content (mg) | Average | RSD |
|---|---|---|---|---|---|
| A | CBD | Top | 8.85 | 8.6 | 2.7 |
| | | Bottom | 8.46 | | |
| | | Mid height | 8.43 | | |
| | THC | Top | 8.41 | 8.1 | 3.1 |
| | | Bottom | 8.02 | | |
| | | Mid height | 7.94 | | |
| C | CBD | Top | 8.92 | 9.0 | 0.3 |
| | | Bottom | 8.96 | | |
| | | Mid height | 8.98 | | |
| | THC | Top | 8.32 | 8.4 | 0.6 |
| | | Bottom | 8.34 | | |
| | | Mid height | 8.41 | | |

The results confirm that dissolving the inner film forms a homogeneous emulsion

### Example 5: Micelles Size Distribution

The size of the micelles after dissolving inner films containing drug emulsion, was tested using the following method:

280 mg of inner film A of Example 1, and 280 mg of inner film C of Example 2 were each dissolved in 250 ml phosphate buffer pH=4.5, using magnetic stirrer for about 30 minutes to achieve a cloudy emulsion.

The emulsion was tested using a laser diffraction method, with Malvern Mastersizer 3000 system, according to the following procedure:

Temperature: 25° C.

Particle Refractive Index: 1.450

Particle Absorption Index: 0.001

Dispersant Name: Water

Dispersant Refractive Index: 1.330

Scattering Model: Mie

Analysis Model: General Purpose

Laser Obscuration: 2-5%

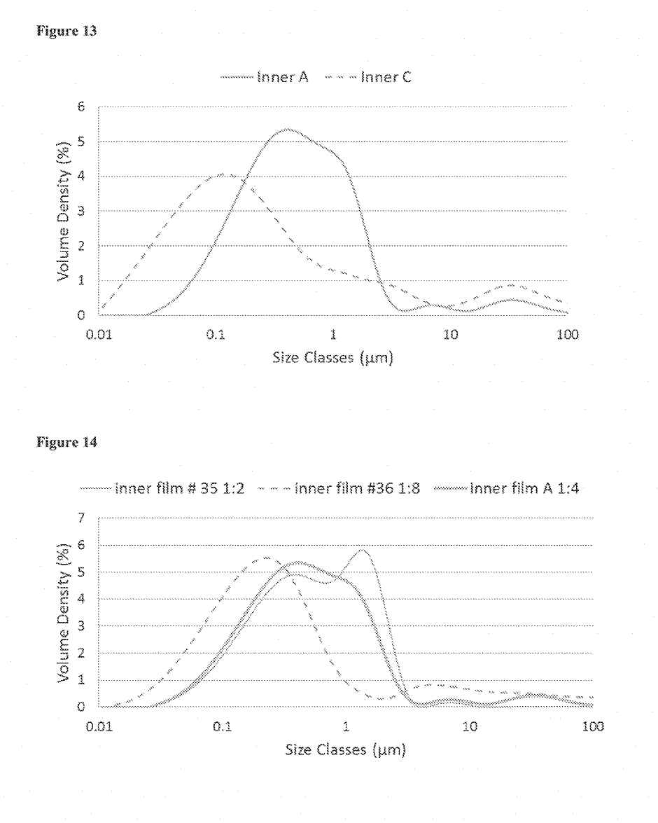

The micelles size distribution results are presented in FIG. 13 and Table 8.

TABLE 8

| Sample | D (10) micron | D (50) micron | D (90) micron |
|---|---|---|---|
| Inner Film A | 0.12 | 0.475 | 1.87 |
| Inner Film C | 0.0342 | 0.188 | 24.6 |

The results in Table 8 are presented in D-values method:
D(10) is the particle size, which 10% of the particles population are equal to or smaller of.
D(50) is the particle size, which 50% of the particles population are equal to or smaller of.
D(90) is the particle size, which 90% of the particles population are equal to or smaller of.

The results confirm that the micelles size of Inner Film C is smaller than that of Inner Film A micelles.

### Example 6: (THC and CBD)/(Labrasol and Kolliphor) Ratio Evaluation

Several inner films, with several (THC and CBD)/(Labrasol and Kolliphor) ratios were tested to analyze the effect of the ratio on the micelles size distribution.

The inners formulations and the (THC and CBD)/(Labrasol and Kolliphor) ratios are summarized in Table 9.

TABLE 9

| % in inner film | Inner film #35 1:2 | Inner film A 1:4 | Inner film #36 1:8 |
|---|---|---|---|
| THC | 4.2 | 3.6 | 1.9 |
| CBD | 4.2 | 3.6 | 1.9 |
| Labrasol | 8.3 | 14.3 | 14.8 |
| Kolliphor EL | 8.3 | 14.3 | 14.8 |
| Klucel EF | 4.7 | 4.0 | 4.1 |
| Klucel GF | 66.2 | 56.7 | 58.8 |
| PEG 400 | 4.2 | 3.6 | 3.7 |
| Total | 100.0 | 100.0 | 100.0 |

The Labrasol/Kolliphor ratio in all formulations was kept constant at 1:1 ratio.

The THC/CBD ratio in all formulations was kept constant at 1:1 ratio.

The inner films were each dissolved in 250 ml phosphate buffer pH=4.5, using magnetic stirrer for about 30 minutes to obtain cloudy emulsion.

The emulsions were tested using laser diffraction method, with Malvern Mastersizer 3000 system, as described in Example 5.

The results of micelles size distribution are presented in FIG. 14 and Table 10.

TABLE 10

| Sample Name | D (10) micron | D (50) micron | D (90) micron |
|---|---|---|---|
| Inner film #35 1:2 | 0.129 | 0.55 | 1.81 |
| Inner film A 1:4 | 0.12 | 0.475 | 1.87 |
| Inner film #36 1:8 | 0.0613 | 0.243 | 7.67 |

The results confirm that there is only minor size change comparing 1:2 to 1:4 (THC+CBD)/(Labrasol+Kolliphor) ratio. In both formulations (Inner film #35 and Inner film A) 90% of the micelles are smaller than 2 microns. The D(50) is about 0.48 micron in 1:4 ratio, and about 0.55 micron on 1:2 ratio.

However, when using a 1:8 (THC+CBD)/(Labrasol+Kolliphor) ratio, a major size change is detected. Two peaks are observed: the first is a main peak of small micelles, with a D(50) of about 0.25 micron and the second is a less significant peak of larger micelles.

### Example 7: (THC and CBD)/(Peceol and Kolliphor) Ratio Effect

Several inner films, with several (THC+CBD)/(Peceol+Kolliphor) ratios were tested to analyze the micelles size distribution.

The formulations and the (THC+CBD)/(Peceol+Kolliphor) ratios are summarized in Table 11.

TABLE 11

| % in inner film | Inner film #37 1:2 | Inner film C 1:4 | Inner film #38 1:8 |
|---|---|---|---|
| THC | 4.2 | 3.6 | 1.9 |
| CBD | 4.2 | 3.6 | 1.9 |
| Kolliphor EL | 11.7 | 20.0 | 20.7 |
| Peceol | 5.0 | 8.6 | 8.9 |
| Klucel EF | 2.7 | 2.3 | 2.4 |
| Klucel GF | 68.1 | 58.4 | 60.6 |
| PEG 400 | 4.2 | 3.6 | 3.7 |
| Total | 100.0 | 100.0 | 100.0 |

The (Peceol+Kolliphor) ratio in all formulations was 3:7. The THC/CBD ratio in all formulations was 1:1.

The inner films were dissolved in 250 ml phosphate buffer pH=4.5, using magnetic stirrer for about 30 minutes to achieve cloudy emulsion.

The emulsion was tested using a laser diffraction method with Malvern Mastersizer 3000 system as described in Example 5.

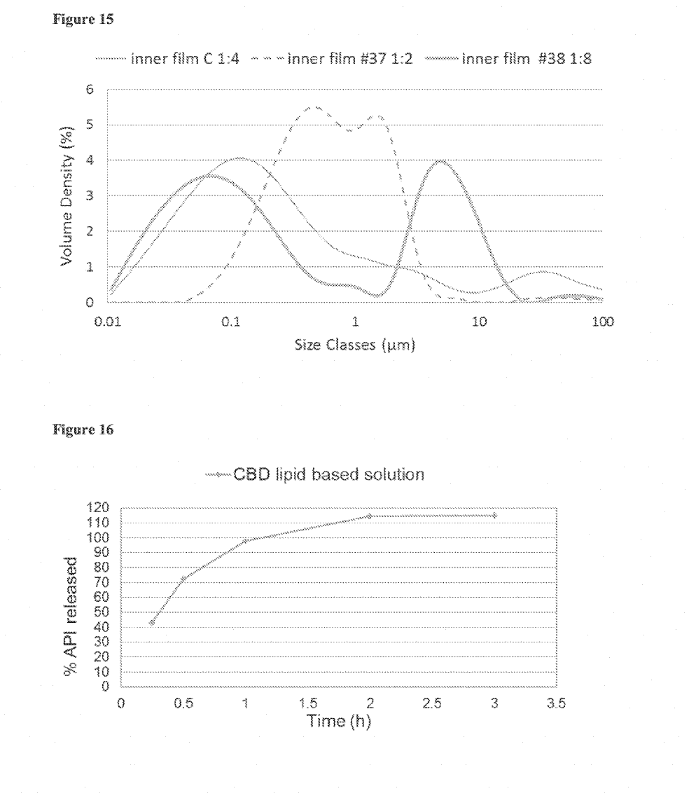

The micelles size distribution results are presented in FIG. 15 and Table 12:

TABLE 12

| Sample | D (10) micron | D (50) micron | D (90) micron |
|---|---|---|---|
| Inner film #37 1:2 | 0.172 | 0.625 | 2.13 |
| Inner film C 1:4 | 0.0342 | 0.188 | 24.6 |
| inner film#38 1:8 | 0.0276 | 0.186 | 8.02 |

As shown in the results, there is major size change comparing 1:2 to 1:4 (THC and CBD/(Peceol and Kolliphor) ratio.

At a 1:4 ratio, 50% of the micelles are less than 0.2 micron, and the D(10) is about 0.035 micron.

At a 1:2 ratio—the micelles are larger—50% of the micelles are less than 0.65 micron, and the D(10) is about 0.17 micron.

When using a 1:8 (THC and CBD)/(Peceol and Kolliphor) ratio, two peaks were observed: the first is a main peak of small micelles, with a D(50) of about 0.18 micron and D(10) of about 0.025 micron. The second is a less significant peak of larger micelles.

### Example 8: A Capsule Containing Immediate Release CBD Lipid Based Solution

An exemplary preparation of a lipid based solution, containing CBD for IR, is presented below. The materials used for preparing the solution and the quantities present in a single capsule containing the solution unit are summarized in Table 13.

TABLE 13

| Solution formulation E | mg per units |
|---|---|
| CBD | 5 |
| Peceol | 30 |
| Kolliphor EL | 65 |

CBD was dissolved in a Peceol and Kolliphor mixture using a magnetic stirrer to obtain a clear solution.

100 mg solution was inserted into a hard gelatin capsule, containing a placebo accordion pill, as an alternative to the supra-outer layer immediate release function.

The final units were tested to define the dissolution profile of the IR fraction.

The dissolution method is as described in Example 1.

The results of the dissolution test are presented in FIG. 16.

### Example 9 (AP-Vitamin E 10 mg Formulation 1)

### Inner Film:

An exemplary preparation of an emulsion containing Vitamin E and the preparation of a film containing Vitamin E micelles, is presented below. The composition of a single inner unit is summarized in Table 14.

TABLE 14

| Inner Film F | mg per inner unit |
|---|---|
| Vitamin E | 10 |
| Labrasol | 40 |
| Kolliphor EL | 40 |
| Klucel EF | 156.5 |
| Klucel GF | 13.5 |
| PEG 400 | 10 |

Vitamin E was dissolved in Labrasol and Kolliphor mixture using magnetic stirrer to achieve clear solution.

In a 0.5-Liter planetary mixer, PEG 400 was dissolved in water.

The Vitamin E clear solution was added to form self-emulsion, Klucel EF, and Klucel GF were added and dissolved, for 30 minutes.

The final emulsion was cast on a silicon-coated PET (Mylar™) web, using a table top casting machine with a knife space of 1200 μm. The cast emulsion was dried in an oven at 60° C. for about 90 minutes. The dried product is a film with a solvent content (based on a loss-on-drying test) value of not more that 6%, and a thickness of about 150 μm.

3 sheets of the dry film were stacked one on top of the other and laminated through a roller. The laminated inner films has a thickness of about 450 μm and a weight per area of about 45.2 mg/cm².

The laminated inner film was punched into 17×39 mm octagon units, each comprising 10 mg Vitamin E.

The outer film was perforated with 14 holes, each with a diameter of 0.85 mm.

The frame, the assembly process and the encapsulation were same as in Example 1.

### Dissolution Profile:

The dissolution method was the same as Example 1.

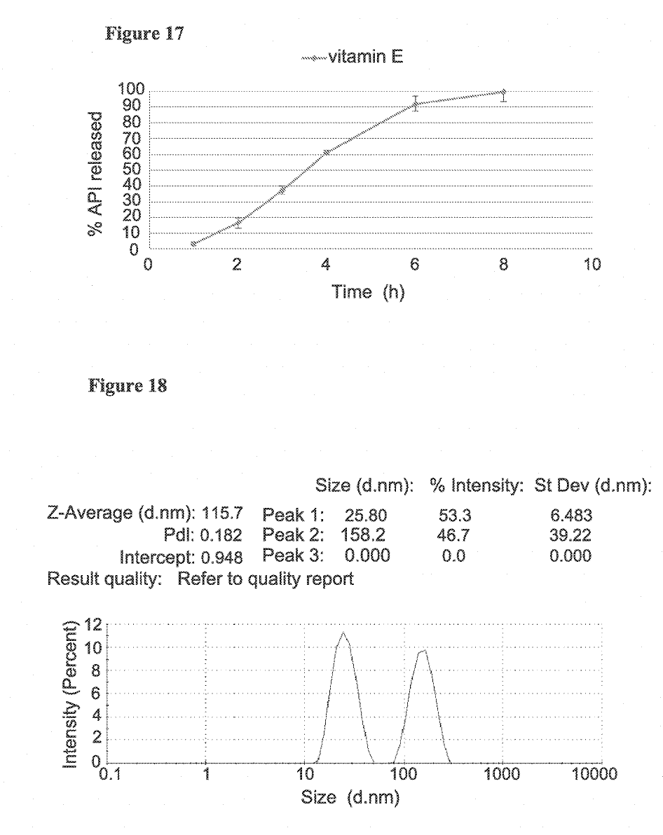

The results of the dissolution test are presented in FIG. 17.

### Micelles Size Distribution:

The size of the micelles after dissolving the inner film contains the emulsion tested using the following method:

280 mg of the inner film F was dissolved in 500 ml SGF, using paddle at 200 RPM for 60 minutes to obtain a clear emulsion.

The emulsion was tested using dynamic light scattering method with Malvern Nano ZSP system according to the following procedure:

Temperature: 37° C.

Material Absorption: 0.01

Dispersant: Water

Dispersant Refractive Index: 1.330

Viscosity: 0.686 Cp

Angle: 173° Backscatter

Cuvette: disposable micro cuvette

The micelles size distribution results (average of four runs) are presented in FIG. 18.

The results confirm that there are two micelle groups in the emulsion.

The size of the first peak is about 0.03 micron and the size of the second peak is about 0.16 micron.

### Example 10: (AP-Vitamin E 10 mg Formulation 2)

### Inner Film:

An exemplary preparation of an emulsion containing Vitamin E and the preparation of a dry film containing Vitamin E micelles, are presented below. The composition of a single inner unit is summarized in Table 15.

TABLE 15

| Inner film G | mg per inner unit |
|---|---|
| Vitamin E | 10 |
| Peceol | 24 |
| Kolliphor EL | 56 |
| Klucel EF | 163.5 |
| Klucel GF | 6.5 |
| PEG 400 | 10 |

Vitamin E was dissolved in a Peceol and Kolliphor mixture using magnetic stirrer to achieve clear solution.

In a 0.5-Liter planetary mixer, PEG 400 was dissolved in water.

The Vitamin E clear solution was added to form self-emulsion, Klucel EF, and Klucel GF were added and dissolved, for 30 minutes.

The final emulsion was cast on a silicon-coated PET (Mylar™) web, using a table top casting machine with a knife space of 1200 μm. The cast emulsion was dried in an oven at 60° C. for about 90 minutes. The dried product is a film with a solvent content (based on a loss-on-drying test) value of not more that 6%, and a thickness of about 170 μm.

3 sheets of the dry film were stacked one on top of the other and laminated through a roller. The laminated inner films has a thickness of about 450 μm and a weight per area of about 45.2 mg/cm².

The laminated inner film was punched into 17×39 mm octagon units, each comprising 10 mg Vitamin E.

The outer film was perforated with 14 holes in each outer, each with a diameter of 0.85 mm.

The frame, the assembly process and the encapsulation were same as in Example 1.

### Dissolution Profile:

The dissolution method is the same as in Example 1.

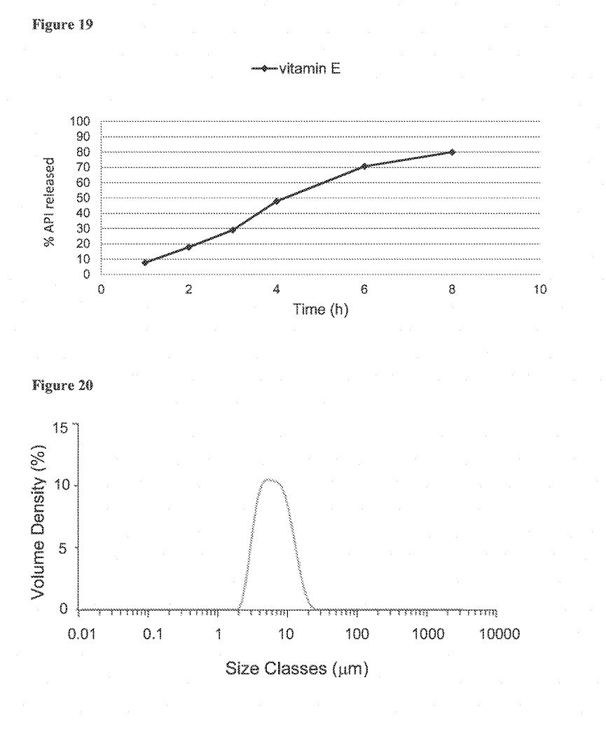

The results of the dissolution test are presented in FIG. 19.

### Micelles Size Distribution:

The size of the micelles after dissolving the inner film contains the emulsion was tested using the Malvern Mastersizer 3000 system method, as described in Example 5.

The results of micelles size distribution are presented in FIG. 20 and Table 16:

TABLE 16

| D(10) micron | D (50)micron | D (90)micron |
|---|---|---|
| 3.33 | 6.21 | 12.1 |

90% of the micelles were in the range of 3-12 micron and the D(50) is 6.21 micron.

### Example 11: (AP-Fenofibrate 5 mg Formulation 1)

### Inner Film:

An exemplary preparation of an emulsion containing Fenofibrate and the preparation of a dry film containing Fenofibrate micelles, are presented below. The composition of a single inner unit is summarized in Table 17.

TABLE 17

| Inner film H | mg per inner unit |
|---|---|
| Fenofibrate | 5 |
| Labrasol | 40 |
| Kolliphor EL | 40 |
| Klucel EF | 156.5 |
| Klucel GF | 13.5 |
| PEG 400 | 10 |

Fenofibrate was dissolved in Labrasol and Kolliphor mixture using magnetic stirrer to obtain a clear solution.

In a 0.5-Liter planetary mixer, PEG 400 was dissolved in water.

The Fenofibrate clear solution was added to form self-emulsion, Klucel EF, and Klucel GF were added and dissolved, for 30 minutes.

The final emulsion was cast on a silicon-coated PET (Mylar™) web, using a table top casting machine with a knife space of 1200 μm. The cast emulsion was dried in an oven at 60° C. for about 90 minutes. The dried product is a film with a solvent content (based on a loss-on-drying test) value of not more that 6%, and a thickness of about 150 μm.

3 sheets of the dry film were stacked one on top of the other and laminated through a roller. The laminated inner films have a thickness of about 400 μm and a weight per area of about 43.6 mg/cm².

The laminated inner film was cut into 17×39 mm octagon units, each comprising 5 mg Fenofibrate.

The outer film was perforated with 14 holes each with a diameter of 0.5 mm.

The frame, the assembly process and the encapsulation were same as in Example 1.

### Dissolution Profile:

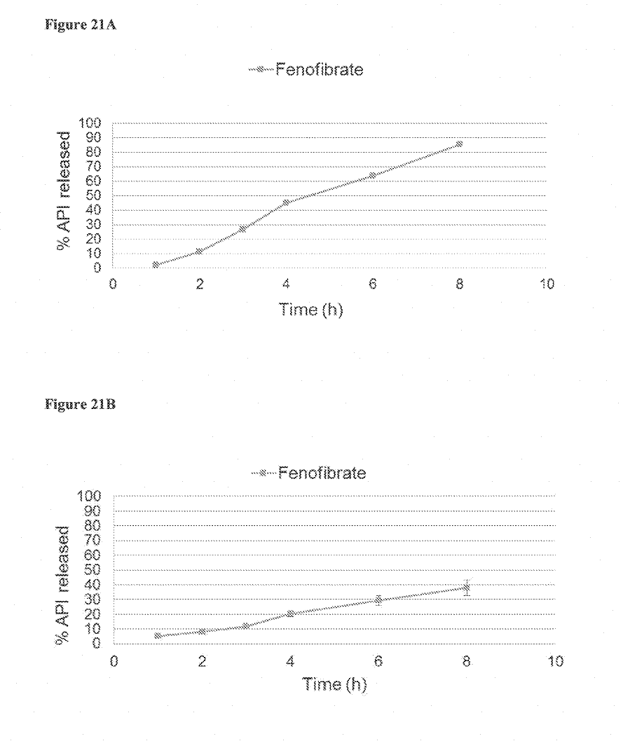

The results of the dissolution test are presented in FIG. 21A.

### Example 12 (AP-Fenofibrate 5 mg Formulation 2)

### Inner Film:

An exemplary preparation of an emulsion containing Fenofibrate and the preparation of a dry film containing Fenofibrate micelles, is presented below. Several inner film sheets are laminated to achieve the desired Fenofibrate dose, as described below. The composition of a single inner unit is summarized in Table 18.

TABLE 18

| Inner film I | mg per inner unit |
|---|---|
| Fenofibrate | 5 |
| Peceol | 24 |
| Kolliphor EL | 56 |
| Klucel EF | 163.5 |
| Klucel GF | 6.5 |
| PEG 400 | 10 |

Fenofibrate was dissolved in Peceol and Kolliphor mixture using magnetic stirrer to obtain a clear solution.

In a 0.5-Liter planetary mixer, PEG 400 was dissolved in water.

The Fenofibrate clear solution was added to form self-emulsion, Klucel EF, and Klucel GF were added and dissolved, for 30 minutes.

The final emulsion was cast on a silicon-coated PET (Mylar™) web, using a table top casting machine with a knife space of 1200 μm. The cast emulsion was dried in an oven at 60° C. for about 90 minutes. The dried product is a film with a solvent content (based on a loss-on-drying test) value of not more that 6%, and a thickness of about 150 μm.

3 sheets of the dry film were stacked one on top of the other and laminated through a roller. The laminated inner films have a thickness of about 400 μm and a weight per area of about 43.6 mg/cm².

The laminated inner film was cut into 17×39 mm octagon units, each comprising 5 mg Fenofibrate.

The outer film was perforated with 14 holes each with a diameter of 0.5 mm.

The frame, the assembly process and the encapsulation were same as in Example 1.

### Dissolution Profile:

The results of the dissolution test are presented in FIG. 21B.

### Non-Emulsified Cannabinoids Formulations

### Example 12—THC Dispersion in Water

### Polymeric Carrier:

An exemplary preparation of a dispersion containing THC and the preparation of a film from the dispersion (polymeric carrier film J, Inner film J) is presented below. The polymeric carrier is prepared by lamination of several film sheets, as described below. The materials that are used for preparing the polymeric carrier dispersion batch and the quantities present in a single polymeric carrier unit are summarized in Table 19.

TABLE 19

| Inner film J with drug (THC) | mg per Inner film unit |
|---|---|
| Water | |
| THC | 15 |
| Povidone K 90 | 40 |
| HPMC K4M | 15 |
| PEG 400 | 20 |

In a 1 Liter mixer, heated to 40° C., 20 g PEG 400 and 40 g Povidone K 90 are dissolved in 400 mL water, for 30 minutes. 15 g THC and 15 g HPMC K4M are added, and dispersed for about 30 minutes.

The dispersion is cast on silicon-coated PET (Mylar™), using a table top casting machine with a knife space of 550-600 μm. The cast dispersion is dried in an oven.

The product is a dry polymeric film containing THC with a loss-on-drying value of not more that 6%, and a thickness of about 60 μm.

The resulting polymeric carrier with THC is cut into 12×39 mm rectangular units, each comprising 15 mg THC.

In another option, two sheets of the dry film are laminated together through a roller. The two laminated sheets form the polymeric carrier which has thickness of about 100 μm and weight of 180 mg/4.68 cm². The resulting polymeric carrier with THC is cut into 12×39 mm rectangular units, each comprising 30 mg THC.

In another option, four sheets of the dry film are laminated together through a roller. The laminated films form the polymeric carrier which has thickness of about 300 μm and weight of 360 mg/4.68 cm². The resulting polymeric carrier with THC is cut into 12×39 mm rectangular units, each comprising 60 mg THC.

### Swelling Membrane (Outer Film):

The composition of the precursor solution for the swelling membrane and, and of the films formed were the same as in Example 1, Table 2.

### Frame Film:

An exemplary preparation of a solution and the preparation of the frame film therefrom are shown below. The materials that are used for preparing the frame solution batch and the quantities present in a single frame unit are summarized in Table 20:

TABLE 20

| Frame | mg per frame unit | % in Solution |
|---|---|---|
| Ethanol Abs. | | 62.8% |
| EUDRAGIT L100 | 111.6 | 22.3% |
| PEG 20,000 | 55.8 | 11.1% |
| PEG 400 | 18.6 | 3.7% |

In a 20-Lilter mixer, heated to 50° C., PEG 400 and PEG 20,000 are dissolved in ethanol, for 60 minutes. Eudragit L100 is added, and dissolved for about 35 minutes.

The solution is cast on silicon-coated PET (Mylar™), using Mathis dryer.

The dried product is a film with a loss-on-drying value of 6.5-8.5% and a weight of 56.0 g/m².

Eight or 12 sheets of the dry film are laminated together through a roller consecutively.

The thickness of the 8 sheets laminated film (the frame layer) is about 360 micrometers and a weight of 448.0 g/m².

The thickness of the 12-sheets laminated film (the frame layer) is about 540 micrometers and a weight of 537.6 g/m².

The laminated frame films are cut to achieve sheets with 16 internal receiving-apertures of 12.5×39 or 20*41 mm for the polymeric carrier units to be inserted.

### Assembly:

The different layers are assembled together and encapsulated, according to the procedure described in Example 1.

### Example 13: CBD—Solution in Ethanol

### Polymeric Carrier (Inner Film):

An exemplary preparation of a dispersion containing CBD, and the preparation of a film therefrom the solution (polymeric carrier film K, inner film K) is presented below. The materials that are used for preparing the polymeric carrier solution batch and the quantities present in a single internal layer unit are summarized in Table 21:

TABLE 21

| Carrier film K | mg per polymeric carrier layer |
|---|---|
| Ethanol Abs. | |
| CBD | 40 |
| Klucel EF | 30 |
| Klucel GF | 3.0 |
| PEG 400 | 20 |

In a 0.5-Liter planetary mixer, 20 g PEG 400, and 30 g Klucel EF, 3 g Klucel GF and 40 g CBD are dissolved in 200 g ethanol, for 30 minutes.

The solution is cast on silicon-coated PET (Mylar™), using a table top casting machine with knife space of 550 μm. The casted solution is dried. The dry film has loss-on-drying of not more that 6%, and thickness of about 50 μm.

The CBD polymeric carrier film is cut into 12×39 mm rectangular units, each comprising 40 mg CBD.

In another option, 4 sheets of the dry film are laminated together through a roller. The resulting laminate, the polymeric carrier, has a thickness of about 200 μm and weight of 380 mg/4.68 cm².

The polymeric carrier laminated films are cut into 12×39 mm rectangular units, each comprising 160 mg CBD.

In another option, 8 sheets of the dry film are laminated together through a roller. The resulting laminated films form the polymeric carrier which has thickness of about 400 μm and weight of 760 mg/4.68 cm².

The polymeric carrier comprising CBD is cut into 12×39 mm rectangular units, each comprising 320 mg CBD.

### Swelling Membrane (Outer Film):

An exemplary preparation of a solution and the preparation of a film therefrom are shown below. The materials that are used for preparing the swelling membrane solution batch and the quantities present in two swelling membrane units are summarized in Table 22:

TABLE 22

| Swelling membrane | mg per 2 swelling membrane units | % in Solution |
|---|---|---|
| IPA | | 90% |
| Eudragit L 100 | 16.25 | 1.75% |
| Eudragit L 100-55 | 26.25 | 1.75% |
| Klucel JF | 41.7 | 4.5% |
| PEG400 | 13.9 | 1.5% |
| Kolliphor 407 | 4.6 | 0.5% |

In a 20 Liter mixer, heated to 50° C., PEG 400 and Kolliphor 407 are dissolved in IPA (isopropanol), for 20 minutes. Klucel J F, Eudragit L 100 and Eudragit L 100-55 are added, and dissolved for about 120 minutes.

The solution is cast on a silicon-coated PET (Mylar™), using a Mathis dryer. The dried product is a film with a loss-on-drying value of not more that 7.5%, and a weight of 13.6 g/m².

Four sheets of the dry film are laminated together through a roller. The resulting laminate (the swelling membrane) has a thickness of about 45 μm and weight of 54.4 g/m².

The swelling membranes are cut to sheets which cover 16 polymeric carrier units, and perforated with 14 holes with a diameter of 0.85 mm or 1.0 mm per unit.

The frame and the assembly process are the same as in Example 1.

### Example 14—(CBD+THC at 1:1 w/w Ratio in Water Dispersion)

### Polymeric Carrier (Inner Film):

An exemplary preparation of a dispersion containing THC+CBD, and the preparation of a film therefrom (Carrier film L) is presented below. The materials that are used for preparing the polymeric carrier dispersion batch and the quantities present in a single polymeric carrier unit are summarized in Table 23:

TABLE 23

| Carrier film L | mg per polymeric carrier unit |
|---|---|
| Water | |
| CBD | 15 |
| THC | 15 |
| HPMC E3 | 30 |
| KLUCEL GF | 1.5 |
| PEG 400 | 20 |

In a 0.5-L planetary mixer, 20 g PEG 400, 30 g HPMC E3 and 1.5 g KLUCEL GF are dissolved in 200 g water, for 30 minutes. 15 g THC+15 g CBD are added and dispersed for about 10 minutes.

The dispersion is cast on silicon-coated PET (Mylar™), using a table top casting machine with knife space of 550 μm. The cast dispersion is dried. The dry film has loss-on-drying of not more that 6%, and thickness of about 110 μm.

Four dry films are laminated together with a roller. The thickness of the resulting laminated film, polymeric carrier, is about 380 μm and its weight is 80 mg/4.68 cm².

The polymeric carrier laminated films are cut into 12×39 mm rectangular units, each comprising 15 mg THC+15 mg CBD.

The swelling membranes are perforated with 14 holes in each membrane, with a diameter of 1.0 mm or 0.3 mm.

The frame and the assembly process, are same as in Example 1.

### Example 15—CBD+THC w/w Ratio of 3:1 in IPA Solution

### Polymeric Carrier (Inner Film):

An exemplary preparation of a solution containing CBD+THC, and the preparation of a film therefrom (Carrier film M) is presented below. The materials that are used for preparing the internal layer solution batch and the quantities present in a single internal layer unit are summarized in Table 24:

TABLE 24

| Carrier film M | mg per internal layer unit |
|---|---|
| IPA | |
| CBD | 45 |
| THC | 15 |
| KLUCEL EF | 30 |
| CMC | 15 |
| PEG 400 | 20 |

In a 0.5-L planetary mixer, 20 g PEG 400, and 30 g KLUCEL EF, 15 g THC+45 g CBD are dissolved in 200 g IPA, for 30 minutes. 15 g CMC is added, and dispersed for about 10 minutes.

The dispersion is cast on silicon-coated PET (Mylar™), using a table top casting machine with knife space of 500 μm. The cast dispersion is dried. The dry film has loss-on-drying of not more that 6%, and thickness of about 120 μm.

Four sheets of the dry film are laminated together through a roller. The laminated film, polymeric carrier, has a thickness of about 480 micrometers and weight of 125 mg/4.68 cm².

The polymeric carrier laminated films are cut into 12×39 mm rectangular units, each comprising 45 mg CBD+15 mg THC

The swelling membranes are perforated with 14 holes in each membrane, with a diameter of 1.0 mm.

The frame and the assembly process are same as in Example 1.

### Example 16-2 Different CR Rates with a 45 Micron Polymeric Layer

### Inert Polymeric Film:

An exemplary preparation of a solution and the preparation of the inert polymeric film (non-drug-containing) therefrom are shown below. The materials that are used for preparing the inert polymeric solution batch and the quantities present in a single inert polymeric film unit are summarized in Table 25:

TABLE 25

| Inert polymeric film | mg per inert polymeric film unit | % in Solution |
|---|---|---|
| Ethanol Abs. | | 62.8% |
| EUDRAGIT L100 | 111.6 | 22.3% |
| PEG 20,000 | 55.8 | 11.1% |
| PEG 400 | 18.6 | 3.7% |

In a 20-Liter mixer, heated to 50° C., PEG 400 and PEG 20,000 are dissolved in ethanol, for 60 minutes. Eudragit L100 is added, and dissolved for about 35 minutes.

The solution is cast on silicon-coated PET (Mylar™), using Mathis dryer.

The dried product is a film with a loss-on-drying value of 6.5-8.5% and a weight of 56.0 g/m² and thickness of 45 micron.

Two layers of carrier film J (comprising THC), one layer of an inert polymeric film and one layer of carrier film K (comprising CBD) are laminated to form the polymeric carrier with THC and CBD of the delivery device. The final laminate thickness is about 200 micron.

The laminated polymeric carrier films are cut into 12×39 mm rectangular units, each comprising 30 mg THC (in carrier film J layer) and 40 mg CBD (in carrier film K layer).

The swelling membranes are perforated with 14 holes in each membrane, with a diameter of 1.0 mm.

The frame and the assembly process, are same as in Example 1.

### Example 17-2 Side by Side CR Polymeric Carriers with Different CR Rates

Four sheets of polymeric carrier laminated films J are cut into 10×41 mm octagonal units, each comprising 30 mg THC.

Two sheets of polymeric carrier laminated films K are cut into 10×41 mm octagonal units, each comprising 40 mg CBD.

The resulting polymeric carriers are inserted, side by side, into 20×41 mm receiving aperture in the frame.

The swelling membranes are perforated with 14 holes in each membrane, with a diameter of 0.3-1.0 mm.

The frame and the assembly process, are same as in Example 1.

### Example 18—THC Delivery Device (with No Frame Member)

### Polymeric Support:

An exemplary preparation of a dispersion containing THC, and the preparation of a film therefrom (polymeric support film N) is presented below. The materials that are used for preparing the polymeric support dispersion batch and the quantities present in a single polymeric carrier unit are summarized in Table 26:

TABLE 26

| polymeric support film N | mg per polymeric support unit |
|---|---|
| Ethanol Abs. | |
| THC | 20 |
| Eudragit 1100-55 | 20 |
| Eudragit 1100 | 10 |
| PEG 400 | 5 |
| KOLLIPHOR P407 | 5 |

In a 0.5-L planetary mixer, 20 g Eudragit 1100-55 and 10 g EUDRAGIT 1100, 5 g Kolliphor, 20 g THC and 5 g PEG 400, are dissolved in 150 g ethanol for 30 minutes. The solution is cast on silicon-coated PET (Mylar™), using a table top casting machine with knife space of 1000 μm. The cast dispersion is dried at 50° C. for 30 minutes. The dry film has loss-on-drying of not more that 8%, and thickness of about 120 μm.

Four sheets of the dry film are laminated together through a roller. The laminated film, polymeric support, has a thickness of about 480 microns.

The polymeric support laminated films are cut into 24×45 mm octagonal units, each comprising 20 mg THC.

### Assembly:

The different layers are assembled together, starting with a first optionally perforated swelling membrane unit, then the polymeric support unit containing THC and then a second optionally perforated swelling membrane unit.

The layered units are folded in an accordion-like configuration using a folding apparatus, and after folding are inserted into a gelatin capsule.

### Example 19—CBD+Solubilizer Delivery Device (with No Frame Member)

### Polymeric Support:

An exemplary preparation of a dispersion containing CBD, and the preparation of a film therefrom (polymeric support film P) is presented below. The materials that are used for preparing the polymeric support dispersion batch and the quantities present in a single polymeric support unit are summarized in Table 27:

TABLE 27

| Polymeric support film P | mg per polymeric support unit |
|---|---|
| Ethanol Abs. | |
| CBD | 160 |
| POVIDONE K90 | 50 |
| CMC 7H3SXF | 10 |
| KOLLIPHOR RH40 | 20 |
| PEG 400 | 30 |

In a 0.5-L planetary mixer, 5 g KOLLIPHOR(solubilizer), 40 g CBD, 7.5 g PEG 400, and 12.5 g POVIDONE K90 are dissolved in 160 g ethanol, for 30 minutes. 2.5 g CMC 7H3SXF is added, and dispersed for about 20 minutes.

The dispersion is cast on silicon-coated PET (Mylar™), using a table top casting machine with knife space of 1000 μm. The cast dispersion is dried at 50° C. for 30 minutes. The dry film has loss-on-drying of not more that 8%, and thickness of about 120 μm.

Four sheets of the dry film are laminated together through a roller. The laminated film, polymeric support, has a thickness of about 580 micrometers.

The polymeric support laminated films are cut into 24×45 mm octagonal units, each comprising 160 mg CBD.

The assembly process is the same as in Example 18.

### Example 20—Device with IR Layer Containing CBD

### Ir Layer:

An exemplary preparation of a solution containing CBD, and the preparation of a film therefrom (IR layer Q) is presented below. The materials used for preparing the IR layer dispersion batch and the quantities present in a single IR layer unit are summarized in Table 28:

TABLE 28

| IR Film Q | mg per IR layer unit |
|---|---|
| IPA | |
| CBD | 20 |
| Eudragit L100-55 | 10 |
| POLOXAMER 407 | 7 |

In a 0.5-L planetary mixer, 20 g CBD, 7 g POLOXAMER 407, and 10 g Eudragit L100-55 are dissolved in 80 g IPA, for 30 minutes.

The solution is cast on silicon-coated PET (Mylar™), using a table top casting machine with knife space of 1000 μm. The cast dispersion is dried at 50° C. for 40 minutes. The dry film has loss-on-drying of not more that 5%, and thickness of about 150 μm.

The IR film Q (IR layer) is cut into 24×45 mm octagonal units, each comprising 20 mg CBD, and is assembled over one swelling membrane of any of the assembled devices of Examples 1 to 19.

### Example 21—Device with IR Layer Containing THC

### Ir Layer:

An exemplary preparation of a dispersion containing THC, and the preparation of a film therefrom (IR film R) is presented below. The materials that are used for preparing the IR layer dispersion batch and the quantities present in a single IR layer unit are summarized in Table 29:

TABLE 29

| IR film R | mg per IR layer unit |
|---|---|
| Water | |
| THC | 20 |
| POVIDONE K90 | 8 |
| PEG 400 | 4 |

In a 0.5-L planetary mixer, 4 g PEG 400 and 8 g POVIDONE K90 are dissolved in50 g water for 30 minutes. 20 g THC is added and dispersed for about 20 minutes. The dispersion is cast on silicon-coated PET (Mylar™), using a table top casting machine with knife space of 1000 μm. The cast dispersion is dried at 50° C. for 40 minutes. The dry film has loss-on-drying of not more that 5%, and thickness of about 150 μm.

The IR layer film R is cut into 24×45 mm octagonal units, each comprising 20 mg THC, and is assembled over one swelling membrane of any of the assembled devices of Examples 12 to 19.

### Example 22—Delivery Device with Added Basic Substance for Acid Protection

### Polymeric Carrier (Inner Film):

An exemplary preparation of a dispersion containing THC+CBD, and the preparation of a film therefrom (Carrier film S) is presented below. The materials that are used for preparing the polymeric carrier dispersion batch and the quantities present in a single polymeric carrier unit are summarized in Table 30:

TABLE 30

| Carrier film S | mg per polymeric carrier unit |
|---|---|
| Water | |
| CBD | 20 |
| THC | 10 |
| Calcium hydroxide | 10 |
| HPMC E3 | 30 |
| KLUCEL GF | 15 |
| PEG 400 | 20 |

In a 0.5-L planetary mixer, 20 g PEG 400, 30 g HPMC E3 and 15 g KLUCEL GF are dissolved in water, for 30 minutes. 10 g calcium hydroxide, 10 g THC and 20 g CBD are added, and dispersed for about 10 minutes.

The dispersion is cast on silicon-coated PET (Mylar™), using a table top casting machine with knife space of 550 μm. The cast dispersion is dried at 70° C. for 40 minutes. The dry film has loss-on-drying of not more that 6%, and thickness of about 110 μm.

Four dry films are laminated together with a roller. The thickness of the laminated film, polymeric carrier, is about 380 μm and its weight is 105 mg/4.68 cm². The polymeric carrier laminated films are cut into 12×39 mm rectangular units, each comprising 10 mg THC+20 mg CBD.

The swelling membranes are perforated with 14 holes in each membrane, with a diameter of 1.0 mm or 0.3 mm.

The frame and the assembly process, are same as in Example 12.

### Example 23: Pharmacokinetic Clinical Study with Tested AP THC/CBD Formulations According to the Present Disclosure

A single-dose, randomized, crossover study is conducted, to compare the safety, tolerability and pharmacokinetics of controlled release Accordion Pill™ comprising mixture of THC and CBD (hereafter AP-THC/CBD) in healthy adult volunteers.

The administered tested formulations are formulation in accordance with the present disclosure, and contain a mixture of CBD and THC at various ratios therebetween, for example at a 1:1 ratio (w/w), contained in said polymeric carrier, respectively polymeric support, in emulsified form or in non-emulsified form. In specific tested formulations the delivery device comprises IR layers, as disclosed herein.

Informed healthy volunteers are included in the clinical study according to pre-determined admission criteria.

Subjects are randomly assigned to different treatment groups. In each dosing period, on morning of administration day, subjects are administered with each tested formulation according to their group. Drug administration is followed by PK blood sampling and AE (adverse event) monitoring for the next 24 hours, at specified time points. An End-of Study (EOS)/Safety Follow-up visit takes place 7-10 days after the last dose of study treatment.

Blood samples to determine plasma concentrations of THC, 11-OH THC and CBD are collected at the pre-determined time points, pre-dose (within 90 min before dosing), 0.5, 1, 1.5, 2, 2.5, 3, 3.5, 4, 5, 6, 8, 10, 12, 18 and 24 hours post dose (a total of 16 samples per dosing period). The following PK parameters are estimated for each subject's plasma THC, 11-OH THC and CBD concentrations: Cₘₐₓ, Tₘₐₓ, AUC₀₋ₜ, AUC_{0-inf}, Kₑₗ, T_{lag} and T_{1/2}. Additional PK parameters can be calculated if deemed necessary.

We claim:

1. A gastro-retentive drug delivery device for oral administration, the device being configured for unfolding from a folded configuration for oral intake to an unfolded configuration for gastric retention, the device comprising:
(a) a drug-containing layer comprising a polymeric carrier, said carrier comprising at least one film-forming polymer and at least one emulsified drug; and
(b) a polymeric frame member configured for imparting mechanical strength to the device sufficient to enable, upon unfolding of the device, the preservation of said unfolded configuration to provide gastric retention, said polymeric frame member accommodating said emulsified drug-containing layer; and
(c) one or two polymeric swelling membranes each covering at least in part one of the two faces of the emulsified drug-containing layer accommodated within said frame member, at least one said swelling membranes optionally comprising orifices.

2. A gastro-retentive drug delivery device of claim 1, wherein said at least one emulsified one drug is in the form of an emulsion of said drug in a pharmaceutically acceptable emulsifying agent.

3. A gastro-retentive drug delivery device of claim 2, wherein said emulsifying agent is at least one oil, glyceride, water insoluble surfactant, water soluble surfactant or co-solvent or any mixture of at least two thereof.

4. A gastro-retentive drug delivery device of claim 2 or claim 3, wherein the weight ratio between said film forming polymer and said emulsion is from about 1:2 to about 20:1.

5. A gastro-retentive drug delivery device of claim 2 or claim 3, wherein the weight ratio between said at least one pharmaceutically active drug and said emulsifying agent is from about 2:1 to about 1:20.

6. A gastro-retentive drug delivery device of any one of claim 1, wherein said at least one drug has log P>2.

7. A gastro-retentive drug delivery device for oral administration according to any one of claims 1 to 6, wherein said at least one drug is a pharmaceutically active cannabinoid or a mixture of at least two pharmaceutically active cannabinoids or a pharmaceutically active cannabis extract.

8. A gastro-retentive drug delivery device of any one of claims 1 to 7, further optionally comprising at least one emulsified drug-containing polymeric layer for immediate release (IR) of said at least one drug (IR layer) covering at least in part one said swelling membrane, said at least one IR layer comprising (1) at least one pharmaceutically acceptable film forming polymer and (2) at least one pharmaceutically active emulsified drug.

9. A gastro-retentive drug delivery device of claim 8, comprising two said drug-containing IR layers, each said IR layer covering at least in part one said swelling membrane.

10. A gastro-retentive drug delivery device of any one of claims 1 to 9, wherein said at least one pharmaceutically active emulsified drug and said at least one film forming polymer are distributed essentially homogeneously throughout the said polymeric carrier.

11. A gastro-retentive drug delivery device of any one of claims 8 to 10, wherein said at least one pharmaceutically active emulsified drug comprised in said at least one IR layer and said at least one film forming polymer comprised in said at least one IR layer are distributed essentially homogeneously throughout said at least one IR layer.

12. A gastro-retentive drug delivery device of any one of claims 1 to 11, wherein said at least one film forming polymer is selected from polymers that are water-soluble and polymers that are partially or completely soluble in both water and organic solvents, and any mixture of at least two thereof.

13. A gastro-retentive drug delivery device of any one of claims 1 to 12, wherein said polymeric carrier further optionally comprises at least one of a pharmaceutically acceptable plasticizer and a pharmaceutically acceptable antioxidant.

14. A gastro-retentive drug delivery device of any one of claims 1 to 13, wherein said polymeric carrier further comprises at least one pharmaceutically acceptable swelling polymer.

15. A gastro-retentive drug delivery device of any one of claims 1 to 14, wherein said at least one film forming polymer is any one of povidone, copovidone, hydroxypropyl cellulose, polyethylene oxide, amino-methacrylate copolymer NF, hydroxypropyl methylcellulose, hydroxyethyl cellulose, carboxymethyl cellulose, polyvinyl alcohol-polyethylene glycol graft copolymer and any combination of at least two thereof.

16. A gastro-retentive drug delivery device of any one of claims 13 to 15, wherein said plasticizer is any one of polyethylene glycols, citrate esters, phthalate esters, glyceryl esters, short-chain triglycerides, medium-chain triglycerides, long-chain triglycerides, olive oil, hydrogenated castor oil, triacetin, glyceryl stearate, glyceryl behenate, dibutyl sebacate, aliphatic alcohols, fatty acids, pegylated aliphatic alcohols and pegylated fatty acids, phospholipids, sorbitan derivatives, polysorbates, poloxamers, hydrogenated castor oil derivatives, glycerin, propylene glycol, and a combination of at least two thereof.

17. A pharmaceutical gastro-retentive drug delivery device of claim any one of claims 14 to 16, wherein the swelling polymer is any one of hydroxypropyl methylcellulose, hydroxypropyl cellulose, hydroxyethyl cellulose, polyethylene oxide, carboxymethyl cellulose, a gum, a protein, and any combination of at least two thereof.

18. A gastro-retentive drug delivery device of any one of claims 8 to 17, wherein said at least one IR layer further comprises at least one of a filler, a surface-active material, a disintegrant, antioxidant or a combination of any two thereof.

19. A gastro-retentive drug delivery device of any one of claims 1 to 18, wherein said swelling membranes each comprises at least one polymeric combination of a soluble polymer and a polymer which is not instantly soluble in gastric medium.

20. A gastro-retentive drug delivery device of claim 19, wherein said soluble polymer is any one of hydroxypropyl cellulose, gelatin, hydroxypropylmethyl cellulose, hydroxyethyl cellulose, carboxymethyl cellulose and polyethylene oxide.

21. A gastro-retentive drug delivery device of any one of claim 19 or 20, wherein said polymer which is not instantly soluble in gastric fluid comprised in said swelling membrane is any one of methacrylic acid copolymer NF, cellulose acetate phthalate, hydroxypropyl methylcellulose phthalate, hydroxypropyl methylcellulose acetate succinate, polyvinyl acetate phthalate or any suitable mixture of at least two thereof.

22. A gastro-retentive drug delivery device of any one of claims 8 to 21, wherein said two IR layers further comprise at least one material that is a plasticizer, a filler, a surface-active material, disintegrant, antioxidant, or any combination of at least two thereof.

23. A gastro-retentive drug delivery device of claim 22, wherein said plasticizer in said IR layers is any one of a polyethylene glycols, citrate esters, phthalate esters, glyceryl esters, short-chain triglycerides, medium-chain triglycerides, long-chain triglycerides, olive oil, hydrogenated castor oil, triacetin, glyceryl stearate, glyceryl behenate, dibutyl sebacate, aliphatic alcohols, fatty acids, pegylated aliphatic alcohols and pegylated fatty acids, phospholipids, sorbitan derivatives, polysorbates, poloxamers, hydrogenated castor oil derivatives, glycerin, propylene glycol, and a combination of at least two thereof.

24. A gastro-retentive drug delivery device of claim 22, wherein said disintegrant in said IR layers is any one of microcrystalline cellulose, crospovidone, croscarmellose, starch and its derivatives, polacrilin, or a mixture of any two thereof.

25. A gastro-retentive drug delivery device of any one of claims 1 to 24, wherein said polymeric frame member comprises at least one polymer that is not instantly soluble in gastric fluid.

26. A gastro-retentive drug delivery device of claim 25, wherein said polymer that it not instantly soluble in gastric fluid comprised in said polymeric frame member is a degradable enteric polymer which is substantially insoluble at pH less than 5.5.

27. A gastro-retentive drug delivery device of claim 25 or claim 26, wherein said frame member further comprises a plasticizer.

28. A gastro-retentive drug delivery device of any one of claims 25 to 27, wherein said polymer that is not instantly soluble in gastric fluid comprised in said polymeric frame member is any one of cellulose acetate phthalate, hydroxypropyl methylcellulose phthalate, hydroxypropyl methylcellulose acetate succinate, polyvinyl acetate phthalate and methacrylic acid copolymer NF, and any suitable mixture of at least two thereof.

29. A gastro-retentive drug delivery device of any one of claims 25 to 28, wherein polymer that it not instantly soluble in gastric fluid comprised in said polymeric frame member is a methacrylic acid copolymer NF.

30. A gastro-retentive drug delivery device of any one of claims 27 to 29, wherein said plasticizer is any one of a polyethylene glycol, or a mixture of two or more polyethylene glycols of different molecular weight, such as any of PEG 200, PEG 300, PEG 400, PEG 540, PEG 600, PEG 800, PEG 1000, PEG 1450, PEG 1540, PEG 3350, PEG 4000, PEG 4500, PEG 6000 and PEG 8000 and PEG 20000, and wherein said plasticizer optionally includes a poloxamer, medium-chain triglycerides, glycerin, glyceryl esters, a polysorbate, a sorbitan derivative, citric acid esters, dibutyl sebacate, an aliphatic alcohol, such as cetyl alcohol, a fatty acid, such as stearic acid, propylene glycol or a combination of the above, preferably the plasticizer is a polyethylene glycol, and a mixture of two or more PEGs with different molecular weight thereof, for example a mixture of PEG 400 and PEG 20,000.

31. A gastro-retentive drug delivery device of any one of claims 1 to 30, wherein said orifices are provided on one of said swelling membranes.

32. A gastro-retentive drug delivery device of any one of claims 1 to 30, wherein said orifices are provided on both said swelling membranes.

33. A gastro-retentive drug delivery device of any one of claims 1 to 31, wherein said device further comprises an anti-adhesion layer covering at least one said swelling membrane.

34. A gastro-retentive drug delivery device of any one of claims 8 to 31, wherein said device further comprises an anti-adhesion layer covering at least in part said at least one IR layer.

35. A gastro-retentive drug delivery device of any one of claims 1 to 35, wherein said at least one swelling membrane comprises a suitable number of identical or different said orifices, and each said orifice has one or more of suitable dimensions, suitable distribution pattern and/or suitable shape.

36. A gastro-retentive drug delivery device of any one of claims 1 to 34, wherein said orifices are provided on both said swelling membranes and wherein in said orifices of one said swelling membrane are staggered with respect to said orifices of the other said swelling membrane.

37. A gastro-retentive drug delivery device of any one of claims 1 to 36, wherein each said swelling membrane comprises from 2 to 24, specifically from 8 to 24 of said orifices.

38. A gastro-retentive drug delivery device of any one of claims 1 to 37, wherein each said orifice has diameter or width of between 0.3 mm and 2.5 mm.

39. A gastro-retentive drug delivery device of any one of claims 1 to 38, wherein said two swelling membranes are co-extensive with said drug-containing layer.

40. A gastro-retentive drug delivery device of any one of claims 1 to 37, wherein said emulsified drug is released from the said device in emulsified form.

41. A gastro-retentive drug delivery device of any one of claims 1 to 40, wherein said drug is a pharmaceutically active cannabinoid or a mixture of at least two pharmaceutically active cannabinoids or a pharmaceutically acceptable cannabis extract.

42. A pharmaceutical dosage unit comprising a gastro-retentive drug delivery device as defined in any one of claims 1 to 41 and a capsule, wherein said drug delivery device in its folded configuration is contained within said capsule.

43. A pharmaceutical dosage unit comprising a gastro-retentive drug delivery device as defined in any one of claims 1 to 7, 10, 12 to 17, 19, 20, 25 to 33 and 35 to 41 and a capsule, wherein said drug delivery device in its folded configuration is contained within said capsule, said capsule further containing an emulsion of said at least one drug in a pharmaceutically acceptable emulsifying agent.

44. The pharmaceutical dosage unit of any one of claim 42 or 43, wherein said delivery device comprises a therapeutically effective amount of said at least one emulsified drug.

45. A pharmaceutical dosage unit of any one of claims 39 to 44, wherein said emulsified drug is at least one emulsified pharmaceutically active cannabinoid or a pharmaceutically active cannabis extract.

46. The pharmaceutical dosage unit of any one of claims 40 to 45, wherein the delivery device comprises a total of from about 1 to about 350 mg of said at least one pharmaceutically active cannabinoid or mixture of at least two pharmaceutically active cannabinoids or pharmaceutically active cannabis.

47. A pharmaceutical dosage unit of claim 46, wherein said at least one emulsified pharmaceutically active cannabinoid or emulsified mixture of at least two pharmaceutically active cannabinoids or pharmaceutically active cannabis extract is distributed between said polymeric carrier and said at least one IR layer.

48. A pharmaceutical dosage unit of any one of claims 43 to 46, wherein the delivery device comprises a total of from about 1 to about 350 mg of said emulsified pharmaceutically active cannabinoid or emulsified mixture of at least pharmaceutically active two cannabinoids or pharmaceutically active cannabis extract, distributed between said polymeric carrier and said emulsion of said cannabinoid/s in said oil contained in said capsule.

49. A pharmaceutical dosage unit of any one of claims 6 to 48, wherein said emulsified mixture of at least two pharmaceutically active cannabinoids comprises THC and CBD at a ratio of from about 20:1 to about 1:20.

50. A pharmaceutical dosage unit of any one of claims 45 to 49, wherein the weight ratio between said film forming polymer and said at least one emulsified pharmaceutically active cannabinoid or pharmaceutically active cannabis extract is from about 1:2 to about 20:1.

51. A pharmaceutical dosage unit of any one of claims 45 to 50, wherein the ratio between said pharmaceutically active cannabinoid or mixture of at least pharmaceutically active two cannabinoids or pharmaceutically active cannabis extract and the emulsifying agent in which they are emulsified is between 2:1 to 1:20.

52. A pharmaceutical dosage unit of claim 46, 47 or 48, wherein the delivery device comprises a total of from about 1 to about 350 mg of a mixture of THC and CBD, distributed between said polymeric carrier and said at least one IR layer at a ratio of from about 1:10, to about 10:1, wherein the ratio THC:CBD in said polymeric carrier and in said at least one IR layer which can be the same or different is from about 1:20 to about 20:1.

53. A pharmaceutical dosage unit of any one of claims 45 to 52, wherein said polymeric carrier comprises one specific cannabinoid, or a mixture of at least two specific cannabinoids, at a suitable ratio therebetween, and said at least one IR layer, respectively said drug emulsion in said capsule, comprises the same or different one specific cannabinoid or mixture of said at least two specific cannabinoids at a suitable ratio therebetween, wherein the ratio between the at least two cannabinoids in said polymeric carrier and in said at least one IR layer, respectively said drug emulsion in said capsule, is the same or different.

54. A gastro-retentive drug delivery device as defined in any one of claims 7 to 41 or a pharmaceutical dosage unit as defined in any one of claims 42 to 53, for use in a method for any one of treating, alleviating and preventing worsening of a disease, disorder or condition responsive to cannabinoid therapy in a subject in need, said method comprising orally administering to said patient said gastro-retentive drug delivery device or pharmaceutical dosage unit.

55. The gastro-retentive drug delivery device for use or pharmaceutical dosage unit for use as defined in claim 54, wherein said disease, disorder or condition responsive to cannabinoid therapy is any one of anorexia associated with weight loss in patients with AIDS, nausea and vomiting associated with cancer chemotherapy, pain, anxiety, depression, muscle spasticity, arthritis and rheumatism, multiple sclerosis and other neuromuscular inflammatory disorders, inflammatory bowel diseases such as Crohn's disease and colitis, post-traumatic stress disorder (PTSD) or epileptic seizures, Parkinson's disease, spinal cord injury, fibromyalgia, Alzheimer's disease and dementia or any other condition responsive to cannabinoid therapy.

56. The gastro-retentive drug delivery device for use or pharmaceutical dosage unit for use as defined in claim 54 or claim 55, wherein said administration is once or twice daily or three times a day.

57. The gastro-retentive drug delivery device for use or pharmaceutical dosage unit for use as defined in claim 54 or claim 55, wherein said administration is chronic.

58. A method for any one of treating, alleviating and preventing worsening of a disease, disorder or condition responsive to cannabinoid therapy in a subject in need, said method comprising orally administering to said patient a gastro-retentive drug delivery device as defined in any one of claims 7 to 41 or a pharmaceutical dosage unit as defined in any one of claims 42 to 53.

59. A method of claim 58, wherein said disease, disorder or condition responsive to cannabinoid therapy is any one of anorexia associated with weight loss in patients with AIDS, nausea and vomiting associated with cancer chemotherapy, pain, anxiety, depression, muscle spasticity, arthritis and rheumatism, multiple sclerosis and other neuromuscular inflammatory disorders, inflammatory bowel diseases such as Crohn's disease and colitis, post-traumatic stress disorder (PTSD) or epileptic seizures, Parkinson's disease, spinal cord injury, fibromyalgia, Alzheimer's disease and dementia or any other condition responsive to cannabinoid therapy.

60. A method for providing a subject in need thereof with stable therapeutically effective plasma level of at least one cannabinoid or mixture of at least two cannabinoids and/or active metabolites thereof over a prolonged period of time, said method comprising orally administering to said patient a gastro-retentive drug delivery device as defined in any one of claims 7 to 41 or a pharmaceutical dosage unit as defined in any one of claims 42 to 53.

61. The method of any one of claims 58 to 60, wherein said administration is once or twice daily or three times a day.

62. The method of any one of claims 58 to 60, wherein said administration is chronic.

63. A method of increasing the oral absorption time of a cannabinoid in a subject in need thereof, by administering to said subject a gastro-retentive device as defined in any one of claims 7 to 41 or a pharmaceutical dosage unit as defined in any one of claims 42 to 53.

64. A method of increasing the absorption time of an active pharmaceutical ingredient (API) having log P>2 in a subject in need thereof, by administering to said subject a gastro-retentive device as defined in any one of claims 6 to 41 or a pharmaceutical dosage unit as defined in any one of claims 42 to 53.

65. A gastro-retentive drug delivery dosage form for oral intake, having a first configuration for oral intake and a second configuration for gastric retention, the device comprising a controlled release functional member comprising a drug in an emulsified form.

66. A gastro-retentive drug delivery device of claim 65, further optionally comprising a functional member for immediate release of an emulsified drug which is identical to or different from said drug contained in said controlled release functional member.

67. A gastro-retentive drug delivery device of claim 66 or claim 67, wherein said device ingested when in said first configuration is configured to assume said second configuration upon exposure to gastric fluids.

68. A gastro-retentive drug delivery device of any one of claims 65 to 67, configured for enabling the preservation of said second configuration to provide gastric retention.

69. A gastro-retentive drug delivery device of any one of claims 66 to 768, comprising means for preservation of said second configuration provide gastric retention.

70. A gastro-retentive drug delivery device of any one of claims 66 to 69, wherein said drug is released from said device in a controlled rate of release, or combined controlled rate and immediate rate of release.

71. A gastro-retentive drug delivery device of any one of claims 65 to 70, wherein said drug is emulsified in a pharmaceutically acceptable emulsifying agent.

72. A gastro-retentive drug delivery device of claim 71, wherein said pharmaceutically acceptable emulsifying agent is at least one oil, glyceride, water insoluble surfactant, water soluble surfactant or co solvent, or any mixture of at least two thereof.

73. A gastro-retentive drug delivery device of any one of claims 64 to 72, wherein said emulsified drug is released in emulsified form.

74. A gastro-retentive drug delivery device of any one of claims 64 to 73, wherein said drug is a drug having log P>2.

75. A gastro-retentive drug delivery device according to any one of claims 66 to 74, wherein said drug is at least one pharmaceutically active cannabinoid and/or cannabis extract.

76. A gastro-retentive drug delivery device according to any one of claim 64 to 75, wherein said device in its said first configuration for oral intake is contained in a capsule.

77. A gastro-retentive drug delivery device according to any one of claims 65 to 76, wherein said device in its said first configuration for oral intake is contained in a capsule, said capsule further containing an emulsion in a pharmaceutically acceptable emulsifying agent of at least one pharmaceutically active drug which is identical to or different from said at least one drug in said controlled release functional member.

78. A pharmaceutical dosage unit for oral administration of a pharmaceutically active cannabinoid or a mixture of at least two pharmaceutically active cannabinoids or cannabis extract, comprising:
(A) a gastro-retentive cannabinoid delivery device, the device being configured for unfolding from a folded configuration for oral intake to an unfolded configuration for gastric retention, the device comprising:
(a) a cannabinoid-containing layer comprising a polymeric carrier, said carrier comprising at least one film forming polymer and at least one pharmaceutically active cannabinoid or cannabinoid-releasing extract formulation; and
(b) a polymeric frame member configured for imparting mechanical strength to the device sufficient to enable, upon unfolding of the device, the preservation of said unfolded configuration to provide gastric retention, said polymeric frame member accommodating said cannabinoid-containing layer; and
(c) one or two polymeric swelling membranes each covering at least in part one of the two faces of the cannabinoid-containing layer accommodated within said frame member, at least one said swelling membranes optionally comprising orifices; and
(B) a capsule;
wherein said cannabinoid delivery device in its folded configuration is contained in said capsule.

79. A pharmaceutical dosage unit for oral administration of a pharmaceutically active cannabinoid or a mixture of at least two pharmaceutically active cannabinoids or cannabis extract, comprising:
(A) a gastro-retentive cannabinoid delivery device, the device being configured for unfolding from a folded configuration for oral intake to an unfolded configuration for gastric retention, the device comprising:
(a) a cannabinoid-containing layer comprising a polymeric support which comprises at least one suitable polymer selected from degradable hydrophilic polymers which is not instantly soluble in gastric fluid, degradable enteric polymers substantially insoluble at pH less than 5.5, or any mixture thereof, and at least one pharmaceutically active cannabinoid or cannabinoid-releasing formulation, wherein the polymeric support is configured for imparting mechanical strength to the device sufficient to enable, upon unfolding of the device, the preservation of said unfolded configuration to provide gastric retention; and
(b) one or two polymeric swelling membranes each covering at least in part one of the two faces of the cannabinoid-containing layer, at least one said swelling membranes optionally comprising orifices; and
(B) a capsule;
wherein said cannabinoid delivery device in its folded configuration is contained in said capsule.

80. A pharmaceutical dosage unit according to claim 78 or claim 79, further optionally comprising at least one cannabinoid-containing polymeric layer for immediate release (IR) of the cannabinoid/s (IR layer) covering at least in part one said swelling membrane, said at least one IR layer comprising (1) at least one pharmaceutically acceptable film forming polymer and (2) at least one pharmaceutically active cannabinoid or cannabinoid-releasing formulation.

81. A pharmaceutical dosage unit according to claim 80, comprising two said cannabinoid-containing IR layers, each said IR layer covering at least in part one said swelling membrane.

82. A pharmaceutical dosage unit according to any one of claim 78, 79 or 80, wherein said at least one pharmaceutically active cannabinoid and said at least one film forming polymer are distributed essentially homogeneously throughout the said polymeric carrier.

83. A pharmaceutical dosage unit according to any one of claims 79 to 81, wherein said at least one pharmaceutically active cannabinoid and said at least one suitable polymer are distributed essentially homogeneously throughout said polymeric support.

84. A pharmaceutical dosage unit according to any one of claims 80 to 83, wherein said pharmaceutically active cannabinoid and said at least one film forming polymer are distributed essentially homogeneously throughout said at least one IR layer.

85. A pharmaceutical dosage unit according to any one of claims 78 and 80 to 83, wherein said at least one film forming polymer is selected from polymers that are water-soluble and polymers that are partially or completely soluble in both water and organic solvents, and any mixture of at least two thereof.

86. A pharmaceutical dosage unit according to any one of claims 78 and 80 to 85, wherein said polymeric carrier further optionally comprises at least one of a pharmaceutically acceptable plasticizer, an antioxidant, a solubilizer and a basic substance, such as a pharmaceutically acceptable metal hydroxide, salt or buffer.

87. A pharmaceutical dosage unit according to any one of claims 79 to 85, wherein said polymeric support further optionally comprises at least one pharmaceutically acceptable plasticizer, an antioxidant, a solubilizer and a pharmaceutically acceptable alkaline agent.

88. A pharmaceutical dosage unit according to claim 86 or claim 87, wherein said IR layer further comprises a plasticizer, which is identical or different from said plasticizer comprised in said polymeric carrier, respectively polymeric support.

89. A pharmaceutical dosage unit according to any one of claims 78 and 80 to 88, wherein said polymeric carrier further comprises at least one pharmaceutically acceptable swelling polymer.

90. A pharmaceutical dosage unit according to any one of claims 79 to 99, wherein said polymeric carrier further comprises at least one pharmaceutically acceptable swelling polymer.

91. A pharmaceutical dosage unit according to any one of claims 78 to 90, wherein said at least one film forming polymer is any one of povidone, copovidone, hydroxypropyl cellulose, polyethylene oxide, amino-methacrylate copolymer NF, hydroxypropyl methylcellulose, hydroxyethyl cellulose, carboxymethyl cellulose polyvinyl alcohol-polyethylene glycol graft copolymer and any combination of at least two thereof.

92. A pharmaceutical dosage unit according to any one of claims 86 to 91, wherein said plasticizer is any one of polyethylene glycols, citrate esters, phthalate esters, glyceryl esters, short-chain triglycerides, medium-chain triglycerides, long-chain triglycerides, olive oil, hydrogenated castor oil, triacetin, glyceryl stearate, glyceryl behenate, dibutyl sebacate, aliphatic alcohols, fatty acids, pegylated aliphatic alcohols and pegylated fatty acids, phospholipids, sorbitan derivatives, polysorbates, poloxamers, hydrogenated castor oil derivatives, glycerin, propylene glycol, and a combination of at least two thereof.

93. A pharmaceutical dosage unit according to any one of claims 89 to 92, wherein the swelling polymer is any one of a hydroxypropyl methylcellulose, a hydroxypropyl cellulose, hydroxyethyl cellulose, a polyethylene oxide, a carboxymethyl cellulose, a gum, a protein, and any combination of at least two thereof.

94. A pharmaceutical dosage unit according to any one of claims 80 to 93, wherein the IR layer further comprises at least one of a filler, a surface-active material, a disintegrant, an antioxidant, a lipid, or a combination of any two thereof.

95. A pharmaceutical dosage unit according to any one of claims 78 to 94, wherein said swelling membranes each comprises at least one polymeric combination of a soluble polymer and a polymer which is not instantly soluble in gastric medium.

96. A pharmaceutical dosage unit according to claim 95, wherein said soluble polymer is any one of hydroxypropyl cellulose, gelatin, hydroxypropylmethyl cellulose, hydroxyethyl cellulose, carboxymethyl cellulose and polyethylene oxide.

97. A pharmaceutical dosage unit according to any one of claim 95 or 96, wherein said polymer which is not instantly soluble in gastric fluid comprised in said swelling membrane is any one of methacrylic acid copolymer NF, cellulose acetate phthalate, hydroxypropyl methylcellulose phthalate, hydroxypropyl methylcellulose acetate succinate, polyvinyl acetate phthalate or any suitable mixture of at least two thereof.

98. A pharmaceutical dosage unit according to any one of claims 80 to 97, wherein said two IR layers further comprise at least one material that is a plasticizer, a filler, a surface-active material, an antioxidant, a disintegrant, a lipid, or a combination of at least two thereof.

99. A pharmaceutical dosage unit according to claim 98, wherein said plasticizer in said IR layers is any one of a polyethylene glycols, citrate esters, phthalate esters, glyceryl esters, short-chain triglycerides, medium-chain triglycerides, long-chain triglycerides, olive oil, hydrogenated castor oil, triacetin, glyceryl stearate, glyceryl behenate, dibutyl sebacate, aliphatic alcohols, fatty acids, pegylated aliphatic alcohols and pegylated fatty acids, phospholipids, sorbitan derivatives, polysorbates, poloxamers, hydrogenated castor oil derivatives, glycerin, propylene glycol, and a combination of at least two thereof.

100. A pharmaceutical dosage unit according to claim 98, wherein said disintegrant in said IR layers is any one of microcrystalline cellulose, crospovidone, croscarmellose, starch and its derivatives, polacrylin, or a mixture of any two thereof.

101. A pharmaceutical dosage unit according to any one of claims 98 to 100, wherein said lipid is any one of a polysorbate, a sorbitan derivative, sodium lauryl sulphate, hydrogenated castor oil and its derivatives or a triglyceride.

102. A pharmaceutical dosage unit according to any one of claims 78 and 80 to 101, wherein said polymeric frame member comprises at least one polymer that is not instantly soluble in gastric fluid.

103. A pharmaceutical dosage unit according to claim 102, wherein said polymer that it not instantly soluble in gastric fluid comprised in said polymeric frame member is a degradable enteric polymer which is substantially insoluble at pH less than 5.5.

104. A pharmaceutical dosage unit according to claim 102 or claim 103, wherein said frame member further comprises a plasticizer.

105. A pharmaceutical dosage unit according to any one of claims 102 to 104, wherein said polymer that it not instantly soluble in gastric fluid comprised in said polymeric frame member is any one of cellulose acetate phthalate, hydroxypropyl methylcellulose phthalate, hydroxypropyl methylcellulose acetate succinate, polyvinyl acetate phthalate and methacrylic acid copolymer NF, and any suitable mixture of at least two thereof.

106. A pharmaceutical dosage unit according to any one of claims 102 to 105, wherein polymer that it not instantly soluble in gastric fluid comprised in said polymeric frame member is a methacrylic acid copolymer NF.

107. A pharmaceutical dosage unit according to any one of claims 104 to 106, wherein said plasticizer is any one of a polyethylene glycol, or a mixture of two or more polyethylene glycols of different molecular weight, such as any of PEG 200, PEG 300, PEG 400, PEG 540, PEG 600, PEG 800, PEG 1000, PEG 1450, PEG 1540, PEG 3350, PEG 4000, PEG 4500, PEG 6000 and PEG 8000 and PEG 20000, and wherein said plasticizer optionally includes a poloxamer, medium-chain triglycerides, glycerin, glyceryl esters, a polysorbate, a sorbitan derivative, citric acid esters, dibutyl sebacate, an aliphatic alcohol, such as cetyl alcohol, a fatty acid, such as stearic acid, propylene glycol or a combination of the above, preferably the plasticizer is a polyethylene glycol, and a mixture of two or more PEGs with different molecular weight thereof, for example a mixture of PEG 400 and PEG 20,000.

108. A pharmaceutical dosage unit according to any one of claims 79 to 101, wherein said degradable hydrophilic polymer which is not instantly soluble in gastric fluid comprised in said polymeric support is any one of hydroxypropyl cellulose, hydroxypropylmethyl cellulose, hydroxyethyl cellulose, carboxymethyl cellulose polyvinyl pyrrolidone, polyethylene oxide and methylcellulose.

109. A pharmaceutical dosage unit according to any one of claims 79 to 101, wherein said degradable enteric polymer substantially insoluble at pH less than 5.5 comprised in said polymeric support is any one of polymethacrylate copolymer, cellulose acetate phthalate, hydroxypropylmethyl cellulose acetate succinate or hydroxypropylmethyl cellulose phthalate.

110. A pharmaceutical dosage unit according to any one of claim 87 to 101, 108 or 109, wherein said plasticizer is any one of a polyethylene glycol, or a mixture of two or more polyethylene glycols of different molecular weight, such as any of PEG 200, PEG 300, PEG 400, PEG 540, PEG 600, PEG 800, PEG 1000, PEG 1450, PEG 1540, PEG 3350, PEG 4000, PEG 4500, PEG 6000 and PEG 8000 and PEG 20000, and wherein said plasticizer optionally includes a poloxamer, medium-chain triglycerides, glycerin, glyceryl esters, a polysorbate, a sorbitan derivative, citric acid esters, dibutyl sebacate, an aliphatic alcohol, such as cetyl alcohol, a fatty acid, such as stearic acid, propylene glycol or a combination of the above, preferably the plasticizer is a polyethylene glycol, and a mixture of two or more PEGs with different molecular weight thereof, for example a mixture of PEG 400 and PEG 20,000.

111. A pharmaceutical dosage unit according to any one of claims 79 to 101 and 108 to 110, wherein polymeric support further comprises a filler, a disintegrant, an antioxidant, a surface-active agent, an additional plasticizer and at least one other processing aid.

112. A pharmaceutical dosage unit according to any one of claims 78 to 111, wherein said orifices are provided on one of said swelling membranes.

113. A pharmaceutical dosage unit according to any one of claims 78 to 111, wherein said orifices are provided on both said swelling membranes.

114. A pharmaceutical dosage unit according to any one of claims 78 and 81 to 113, wherein said device further comprises an anti-adhesion layer covering at least one said swelling membrane.

115. A pharmaceutical dosage unit according to any one of claims 80 to 113, wherein said device further comprises an anti-adhesion layer covering at least in part said at least one IR layer.

116. A pharmaceutical dosage unit according to any one of claims 78 to 115, wherein said at least one swelling membrane comprises a suitable number of identical or different said orifices, and each said orifice has one or more of suitable dimensions, suitable distribution pattern and/or suitable shape.

117. A pharmaceutical dosage unit according to any one of claims 78 to 116, wherein said orifices are provided on both said swelling membranes and wherein in said orifices of one said swelling membrane are staggered with respect to said orifices of the other said swelling membrane.

118. A pharmaceutical dosage unit according to any one of claims 78 to 116, wherein each said swelling membrane comprises from 2 to 24, specifically from 8 to 24 of said orifices.

119. A pharmaceutical dosage unit according to any one of claims 78 to 118, wherein each said orifice has diameter or width of between 0.3 mm and 2.5 mm.

120. A pharmaceutical dosage unit according to any one of claims 78 to 119, wherein said two swelling membranes are co-extensive with said cannabinoid-containing layer.

121. A pharmaceutical dosage unit according to any one of claims 78 to 120, wherein the capsule is configured for disintegrating in a gastric environment on exposure thereto.

122. The pharmaceutical dosage unit according to any one of claims 78 to 121, wherein the delivery device comprises a total of from about 1 to about 350 mg of said pharmaceutically active cannabinoid or mixture of at least two cannabinoids.

123. A pharmaceutical dosage unit according to any one of claims 80 to 119, wherein the delivery device comprises a total of from about 1 to about 350 mg of said pharmaceutically active cannabinoid or mixture of at least two cannabinoids, distributed between said polymeric carrier or polymeric support and said at least one IR layer.

124. A pharmaceutical dosage unit according to any one of claims 78 to 121, wherein said pharmaceutically active mixture of at least two cannabinoids comprises THC and CBD at a ratio of from about 1:20 to about 20:1.

125. A pharmaceutical dosage unit according to any one of claims 80 to 122, wherein the delivery device comprises a total of from about 1 to about 350 mg of a mixture of THC and CBD, distributed between said polymeric carrier or polymeric support and said at least one IR layer at a ratio of from about 1:10 to about 10:1, wherein the ratio THC:CBD in said polymeric carrier or polymeric support and in said at least one IR layer is the same or different.

126. A pharmaceutical dosage unit according to claim 125, wherein said polymeric carrier or polymeric support comprises one defined cannabinoid, for example THC or CBD, or a defined mixture of at least two cannabinoids, for example THC and CBD at a suitable ratio therebetween, and said at least one IR layer comprises the same or different one defined cannabinoid or defined mixture of at least two cannabinoids at a suitable ratio therebetween, wherein the ratio between the at least two cannabinoids in said polymeric carrier or polymeric support and in said at least one IR layer is the same or different.

127. A pharmaceutical dosage unit according to claim 78, wherein said polymeric carrier comprises three distinct contiguous laminated polymeric films, a first polymeric film comprising at least one cannabinoid, a second polymeric film comprising at least one cannabinoid and a third polymeric film being a non-drug-containing polymeric film, wherein said third polymeric film is positioned between said first and second polymeric films, and wherein said at least one cannabinoid comprised in said first polymeric film and said at least one cannabinoid comprised in said second polymeric film are the same or different.

128. A pharmaceutical dosage unit according to claim 127, wherein each said first and second polymeric films releases said at least one cannabinoid comprised therein at a controlled release rate, wherein the controlled release rates of said at least one cannabinoid from each said first and second cannabinoids are the similar or different rates of release.

129. A pharmaceutical dosage unit according to claim 79, wherein said polymeric support comprises three distinct contiguous laminated polymeric films, a first polymeric film comprising at least one cannabinoid, a second polymeric film comprising at least one cannabinoid and a third polymeric film being an inert polymeric film, wherein said third polymeric film is positioned between said first and second polymeric films, and wherein said at least one cannabinoid comprised in said first polymeric film and said at least one cannabinoid comprised in said second polymeric film are the same or different.

130. A pharmaceutical dosage unit according to claim 129, wherein each said first and second polymeric films releases said at least one cannabinoid comprised therein at a controlled release rate, wherein the controlled release rates of said at least one cannabinoid from each said first and second cannabinoids are the similar or different rates of release.

131. A pharmaceutical dosage unit for oral administration of a pharmaceutically active cannabinoid or a mixture of at least two pharmaceutically active cannabinoids, comprising a gastro-retentive cannabinoid delivery device folded into a capsule.

132. A pharmaceutical dosage unit according to any one of claims 78, 79, 82, 83, 85-87, 89-93, 95-97, 101-114, 116-122, and 129-131, wherein said capsule further contains an emulsion of said at least one cannabinoid or cannabis extract in a pharmaceutically acceptable emulsifying agent.

133. A pharmaceutical dosage unit according to any one of claims 78 to 132, for use in a method for any one of treating, alleviating and preventing worsening of a disease, disorder or condition responsive to cannabinoid therapy in a patient in need, said method comprising administering to said patient said pharmaceutical dosage unit.

134. A pharmaceutical dosage unit for use according to claim 133, wherein said disease, disorder or condition responsive to cannabinoid therapy is any one of anorexia associated with weight loss in patients with AIDS, nausea and vomiting associated with cancer chemotherapy, pain, anxiety, depression, muscle spasticity, arthritis and rheumatism, multiple sclerosis and other neuromuscular inflammatory disorders, inflammatory bowel diseases such as Crohn's disease and colitis, post-traumatic stress disorder (PTSD) or epileptic seizures, Parkinson's disease, spinal cord injury, fibromyalgia, Alzheimer's disease and dementia or any other condition responsive to cannabinoid therapy.

135. A pharmaceutical dosage unit according to any one of claims 78 to 132, for use in a method for providing a patient in need thereof with stable therapeutically effective plasma level of at least one cannabinoid or mixture of at least two cannabinoids, said method comprising administering to said patient said pharmaceutical dosage unit.

136. A pharmaceutical dosage unit for use according to claim 135, wherein said patient suffers from a condition responsive to cannabinoid therapy disease, which can be any one of anorexia associated with weight loss in patients with AIDS, nausea and vomiting associated with cancer chemotherapy, pain, anxiety, depression, muscle spasticity, arthritis and rheumatism, multiple sclerosis and other neuromuscular inflammatory disorders, inflammatory bowel diseases such as Crohn's disease and colitis, post-traumatic stress disorder (PTSD) or epileptic seizures, Parkinson's disease, spinal cord injury, fibromyalgia, Alzheimer's disease and dementia.

137. The pharmaceutical dosage unit for use according to any one of claims 133 to 136, wherein said administration is once or twice daily or three times a day.

138. The pharmaceutical dosage unit for use according to any one of claims 133 to 136, wherein said administration is chronic.
